SEQUENCE LISTING (24 sequences)

SEQ ID NO: 1 - DNA, 30 bp, Artificial Sequence
  Description of Artificial Sequence Primer
cccccccccccccccgtctagannnnnnnn

SEQ ID NO: 2 - DNA, 39 bp, Artificial Sequence
  Description of Artificial Sequence Primer
cccccccccccccccgtctagatttttttttttttttvn

SEQ ID NO: 3 - DNA, 27 bp, Artificial Sequence
  Description of Artificial Sequence Primer
tcagaattcatgccccccccccccccc

SEQ ID NO: 4 - DNA, 15 bp, Artificial Sequence
  Description of Artificial Sequence Primer
ttttttttttttttt

SEQ ID NO: 5 - DNA, 23 bp, Artificial Sequence
  Description of Artificial Sequence Primer
gatggagccgccgatccacacgg

SEQ ID NO: 6 - DNA, 39 bp, Artificial Sequence
  Description of Artificial Sequence Primer
tttctccttaatgtcacagatctcgaggatttcnnnnnn

SEQ ID NO: 7 - DNA, 36 bp, Artificial Sequence
  Description of Artificial Sequence Primer
gctgaagtggcgaattccgatgcccccccccccccc

SEQ ID NO: 8 - DNA, 30 bp, Artificial Sequence
  Description of Artificial Sequence Primer
ctccttaatgtcacagatctcgaggatttc

SEQ ID NO: 9 - DNA, 25 bp, Artificial Sequence
  Description of Artificial Sequence Primer
ttttttttttttttttttttttttt

SEQ ID NO: 10 - DNA, 28 bp, Artificial Sequence
  Description of Artificial Sequence Primer
cccccccccccccccgtctagannnnnn

SEQ ID NO: 11 - DNA, 32 bp, Artificial Sequence
  Description of Artificial Sequence Primer
cccccccccccccccgtctagannnnnnnnnn

SEQ ID NO: 12 - DNA, 20 bp, Artificial Sequence
  Description of Artificial Sequence Primer
acgattccctgatgaggcag

SEQ ID NO: 13 - DNA, 20 bp, Artificial Sequence
  Description of Artificial Sequence Primer
ccatcttcacgttgagcagg

SEQ ID NO: 14 - DNA, 24 bp, Artificial Sequence
  Description of Artificial Sequence Primer
ctgagacgccatctgtaggcggtg

SEQ ID NO: 15 - DNA, 24 bp, Artificial Sequence
  Description of Artificial Sequence Primer
gtctttggctaccagtccagcagc

SEQ ID NO: 16 - DNA, 20 bp, Artificial Sequence
  Description of Artificial Sequence Primer
aagagaccacacttgtgcgg

SEQ ID NO: 17 - DNA, 20 bp, Artificial Sequence
  Description of Artificial Sequence Primer
aatgtggtgctgagtcgagg

SEQ ID NO: 18 - DNA, 20 bp, Artificial Sequence
  Description of Artificial Sequence Primer
cggtgtccagttccaatacc

SEQ ID NO: 19 - DNA, 20 bp, Artificial Sequence
  Description of Artificial Sequence Primer
ccccatagtccaccaacatg

SEQ ID NO: 20 - DNA, 20 bp, Artificial Sequence
  Description of Artificial Sequence Primer
atgccactctcgtcttcgat

SEQ ID NO: 21 - DNA, 20 bp, Artificial Sequence
  Description of Artificial Sequence Primer
ggaacatcaggaaaagctcc

SEQ ID NO: 22 - DNA, 20 bp, Artificial Sequence
  Description of Artificial Sequence Primer
tacaaggctgaggatgaggc

SEQ ID NO: 23 - DNA, 20 bp, Artificial Sequence
  Description of Artificial Sequence Primer
cttcccgacacttgtcttgc

SEQ ID NO: 24 - DNA, 23 bp, Artificial Sequence
  Description of Artificial Sequence Primer
ctacgtcgccctggacttcgagc

(19) United States
(12) Patent Application Publication
(10) Pub. No.: US 2004/0048816 A1
(43) Pub. Date: Mar. 11, 2004

(54) Restenosis treatment

(76) Inventors: Dietlind Zohlnhöfer, Munchen (DE); Patrick Bauerle, Gauting (DE); Christoph Klein, Munchen (DE); Franz-Josef Neumann, Munchen (DE)

Correspondence Address: FOLEY AND LARDNER, SUITE 500, 3000 K STREET NW, WASHINGTON, DC, 20007, US

(21) Appl. No.: 10/239,655

(22) PCT Filed: Mar. 23, 2001
(86) PCT No.: PCT/EP01/03312

(30) Foreign Application Priority Data
Mar. 24, 2000 (EP) 00106468.2

(51) Int. Cl. A61K 48/00; A61K 39/395; A61M 31/00

(52) U.S. Cl. 514/44; 424/145.1; 604/500

(57) ABSTRACT

The present invention relates to the use of a an inhibitor for the interferon γ signaling pathway for the preparation of a pharmaceutical composition for the treatment or prevention of restenosis. Furthermore, the present invention provides for a method for the identification of an inhibitor of the IFN-γ signaling pathway.

The present invention relates to the use of a an inhibitor for the interferon γ signaling pathway for the preparation of a pharmaceutical composition for the treatment or prevention of restenosis. Furthermore, the present invention provides for a method for the identification of an inhibitor of the IFN-γ signaling pathway.

Several documents are cited throughout the text of this specification. The disclosure content of each of the documents (including any manufacturer's specifications, instructions, etc.) is herewith incorporated by reference.

The success of balloon angioplasty with subsequent stent implantation in the treatment of coronary artery disease is limited by a high rate of restenosis.

Restenosis is defined as the process that occurs at the cellular level within a blood vessel in response to percutaneous treatment for coronary stenosis leading to re-narrowing of the treated vessel. (Topol, “Textbook of Interventional Cardiology”, Saunders Company (1999)). Hereby, three different types of restenosis can be distinguished. First, histologic restenosis which refers to the process that occurs at the cellular level within the vessel and that can be quantified by intravascular ultrasonography. Second, angiographic restenosis, which can be measured either by visual inspection of an angiogram or by quantitative coronary angiography (QCA). Finally, clinical restenosis which refers to the occurrence of clinical events related to restenosis leading to repeat revascularization of the vessel that was initially treated (see, Topol, (1999), loc. cit.)

Examples of restenosis comprise restenosis of coronary arteries, of carotid arteries, of femoralis arteries and of other peripheral blood vessels, restenosis of aorta-coronary vein bypass and restenosis of arterial and veinous bypass(es).

The pathobiology of restenosis is completely different to the pathobiology of atherosclerosis. Atherosclerosis and post-transplant graft atherosclerosis are both characterized by expansion of the arterial intima as a result of the infiltration of mononuclear leukocytes, the proliferation of vascular smooth muscle cells and the accumulation of extracellular matrix (Hansson, Arteriosclerosis 9 (1989), 567-578 and Libby, Transplant. Proc. 21 (1989), 3677-3684 and Ross, Am. Heart J. 138 (1999), S419-S420). Endothelial dysfunction seems to be the major factor in pathophysiology of atherosclerosis and is caused by elevated and modified LDL (low density lipoproteins), free radicals, hypertension, diabetes mellitus genetic alterations, elevated plasma homocystein concentrations and infectious organisms like herpesviruses of Chiamydia pneumoniae. The different forms of injury increase the permeability of the endothelium and its adhesiveness for leukocytes and platelets. If the pathogenic agents are not neutralized or removed effectively, the inflammatory response continues leading to migration and proliferation of smooth muscle cells. In addition, activation of macrophages and monocytes induces the release of hydrolytic enzymes, cytokines, chemokines and growth factors (Mach, Nature 394 (1998), 200-203 and Sultan, Proc. Natl. Acad. Sci. U.S.A 94 (1997), 8767-8772) resulting in further damage and eventually focal necrosis (Groenewegen, Nature 316 (1985), 361-363). In conclusion, proliferation of smooth muscle cells as well as replication of monocyte-derived macrophages and T-cells contribute to the expansion of atherosclerotic lesions. Macrophage accumulation may be associated with increased plasma concentrations of both fibrinogen and C-reactive protein (Haverkate, Lancet 349 (1997), 462-466 and Ridker, N. Engl. J. Med. 336 (1997), 973-979 and Toss, Circulation 96 (1997), 4204-4210), two markers of inflammation supposed to be early signs of atherosclerosis (Berk, Am. J. Cardiol. 65 (1990), 168-172 and Levenson, Arterioscler. Thromb. Vasc. Biol. 15 (1995), 1263-1268 and Ridker, N. Engl. J. Med. 336 (1997), 973-979).

In summary, atheroscieroses is a chronic disease process that progresses over years and is characterized by periods of intense proliferative activity and non-linear growth of many developing plaques at different sites and time points.

Compared to atherosclerosis, the pathobiology of restenosis is different in many aspects. Restenosis is characterized by an abrupt onset and rapid progression with intense proliferative activity followed by stabilization and quiescence. The predominant cellular mechanisms that contribute to restenosis include thrombosis, vascular muscle cell migration and proliferation and adventitial scarring. Neointimal hyperplasia ultimately results from the migration and proliferation of smooth muscle cells and their production of abundant extracellular matrix. 4-14 days after injury smooth muscle cell proliferation occurs. Intimal thickening and extracellular matrix deposition characterize the last phase of pathogenic processes in restenosis. In contrast to atherosclerosis, restenosis is independent of the concentration or composition of plasma lipids and lipid accumulation. The inflammatory process in restenosis is characterized by recruitment and adherence of monocytes in response to intimal injury. There is a significant correlation between the severity of vascular injury and the degree of inflammation and neointima formation (Kornowski, J. Am. Coll. Cardiol. 31 (1998), 224-230 and Rogers, Arterioscler. Thromb. Vasc. Biol. 16 (1996), 1312-1318).

Further, restenosis can be differentiated from atherosclerosis by the development of a restenotic lesion, which differs from that of the de novo lesion formation in atherosclerosis in several ways. As mentioned above, primary atherosclerotic notic lesions develop over weeks to months, probably due to the level of injury that occurs when the plaque ruptures as compared to the ruptures induced by percutaneous interventions. The latter leads to a more intense cellular signal, which results in the different pathogenesis, timing, and treatment effect of the restenotic lesion compared with primary plaque formation. Therefore, restenosis differs from artherosclerosis in the way how injury occurs (e.g., stent implantation), its time of development (weeks to month) and in the cell types involved in said lesion, namely in the main cellular component smooth muscle cells. No foam cells or plaque(s) with lipid rich cores are described in restenosis (Topol, (1999) loc. cit.).

The above mentioned luminal re-narrowing of a coronary artery after balloon angioblasty (restenosis) is a complication which occurs in 30 to 50% of patients. In this context, it is considered that an arterial healing response to injury occurs during revascularization (Ellis, J. Am. Coll. Cardiol. 19 (1992), 275-277). This healing response is attributed to many factors (Topol, (1999), loc. cit.) including the response to vessel injury, thrombus formation at the injury site and an inflammatory response leading to migration of smooth muscle cells (SMCs), an increase in myoinitial proliferation and to excessive extracellular matrix production (Farb, Circulation 99 (1999), 44-52).

Animal models of arterial injury in restenosis have been developed in different species and extensively studied in the past (for review Schwartz in Topol “Textbook of Inventional Cardiology”, (1999) 358-378). Many animal models established in rat, mouse, rabbit and pig have concentrated on the mechanisms of neointimal thickening and attempts to limit neointimal growth by inhibiting smooth muscle cell proliferation. The rat carotid model became a standard for studying smooth muscle cell proliferation after endothelial denudation (Guyton J, Circ. Res. 46 (1980), 625-634) and is well characterized from its molecular biological context. This model provides insights into molecular and genetic mechanisms of the arterial injury response (Clowes, Circ. Res. 67 (1990), 61-67 and Fingerle, Arteriosclerosis 10 (1990), 1082-1087 and Golden, J. Vasc. Surg. 11 (1990), 580-585 and Guyton J, (1980) and Pickering, Circulation 93 (1996), 772-780 and Reidy, Lab Invest 49 (1983), 569-575 and Simons M, (1991) and Simons, Nature 359 (1992), 67-70 and Simons, Jpn. Circ. J. 60 (1996), 1-9) and was used to study the mechanisms of neointima formation and to test substances in regard to their ability to inhibit neointima formation.

The mouse arterial injury model has recently come into use as a model for coronary restenosis. The major advantage of all murine models lies in the great knowledge of the molecular biology of the mouse and allows the application of the knockout technology to identify the role of single genes in the pathological processes of restenosis. Arterial injury in mouse can be achieved by different methods like the use of guidewires (Lindner, Am. J. Pathol. 148 (1996), 427-438 and Lindner, Arterioscler. Thromb. Vasc. Biol. 16 (1996), 1399-1405 and Reidy, Circ. Res. 78 (1996), 405-414), electrical injury (Carmeliet, Thromb. Haemost. 74 (1995), 429-436 and Carmeliet, Am. J. Pathol. 150 (1997), 761-776), polyethylen tubes and atherectomy and represents a good model to study cellular migration, enhanced neointima formation combined with a significant inflammatory response.

The rabbit iliac model (Faxon, Arteriosclerosis 2 (1982), 125-133 and Faxon, Arteriosclerosis 4 (1984), 189-195 and Gertz, Circulation 90 (1994), 3001-3008 and La Veau, Circulation 82 (1990), 1790-1801) has been studied to test restenosis therapies and to understand mechanisms of restenosis. This model uses rabbit iliac artery injury and hypercholesterolemic diet. The very new model of restenosis is the rabbit ear crush injury model characterized by rapidly producing neointima and smooth muscle cell proliferation. Two porcine models of restenosis, the carotid injury and the coronary injury model have also been studied extensively. The coronary arteries of domestic crossbred pigs respond similarly to human coronary arteries after deep injury (Schwartz, J. Am. Coll. Cardiol. 19 (1992), 267-274). The histopathological features of neointima are identical to human restenotic neointima. The porcine coronary models have become the standard by which potential restenosis therapies are applied. Negative trials in the pig have corresponded to negative clinical trials suggesting the specificity of this model.

While there are abundant data from various animal models, only sparse data are available from humans regarding the molecular events following stent implantation and resulting restenotic events (Komatsa, Circulation 98 (1998), 224-233; Kearney, Circulation 95 (1997), 1998-2002).

Up to now, treatment options for restenosis, in particular of restenosis after stent implantations, are limited to surgical management, comprising percutaneous revascularization procedures, preferentially stand-alone balloon angioplasty or the use of intercoronary stents as bail-out devices. Rates of recurrent restenosis after stand-alone balloon angioplasty were reported to be 43% (Serruys, N. Engl. J. Med. 331 (1994), 489-495; Fischman, N. Engl. J. Med. 331 (1994), 496-501). However the use of coronary stents has been accompanied by a new and problematic entity, in-stent restenosis, which currently represents the most important drawback to stent implantation. When diffuse restenosis occurs within a previously implanted stent, preliminary evidence suggests that the long-term efficacy of repeated percutaneous therapy is poor (Topol, (1999), loc. cit.). However, a recent clinical follow up study over 8 months demonstrated that the use of stents coated with sirolimus (rapamycin, a cell-cycle inhibitor) led to minimal neointimal hyperplasia and prevention of in-stent restenosis (Sousa, Circulation 103b (2001), 192-195).

Effective pharmacological intervention for restenosis is lacking. The most advanced compound in clinical development is a monoclonal antibody, “abciximab” (Genetta, Ann. Pharmacother. 30 (1996), 251-257 and Lefkovits, Am. J. Cardiol. 77 (1996), 1045-1051), recognizing glycoprotein IIb/IIIa and being in experimental phase III clinical trials. The compound showed an effect on death rate, myocardial infarction and/or target vessel vascularization at 30 days after surgery (5.3% versus 10.6%), as described in Topol, (1999), loc. cit., however said antibody showed rather disappointing results in intervention of restenosis or restenotic modifications. Similarly, substances like aspirin, ticlopidine, thromboxane A2 inhibitors, prostacyclin, anticoagulants, calcium antagonist, steroids, ACE inhibitors, trapidil, lipid-lowering agents, antioxidants and serotonin antagonists have been tested for prevention and/or treatment of restenosis, however, with rather limited success (Topol, (1999), loc. cit.).

Therefore, there is a need in the art to develop means and methods for the prevention or treatment of restenosis.

The solution to this technical problem is achieved by providing the embodiments characterized in the claims.

Accordingly, the present invention relates to the use of a an inhibitor for the IFN-γ (interferon γ) signaling pathway for the preparation of a pharmaceutical composition for the treatment or the prevention of restenosis.

In the context of the present invention, the term “interferon γ signaling pathway” comprises any part of said pathway from the production and/or release of IFN-γ, the binding of IFN-γ to its ligand, to the following cascade of signaling events (downstream signals) and its involved molecules, the regulation of transcription (involving transcription factors relevant to IFN/IFN-γ signaling), and/or the expression of IFN-γ target genes (or gene products) like, inter alia, APO-2 ligand (TRAIL), PYK2, BAG-1, Pim1, Dap1, IFN-γ-inducible protein, IFN-γ-inducible protein 9-27, CD40, CD13, thrombospondin-1, p47-PHOX, platelet-membrane glycoprotein lib, caspase-1, caspase-8, ICAM-1 or allograft inflammatory factor 1. Therefore, in the context of the present invention, the term “IFN-γ signaling pathway” also comprises target gene products which are regulated by IFN-γ binding to its relevant receptor.

The term “inhibitor” means in accordance with the present invention a compound or a plurality of compounds capable of interfering with, such as downregulating, suppressing or neutralizing the interferon γ signaling pathway e.g. interaction of INF-γ with its corresponding ligand, which may, for example be a INFγ receptor. For example neutralization of IFNγ activity may be induced by binding to a specific antibody or interaction with a ligand as mentioned hereinafter. In accordance with the present invention said inhibitor preferably interacts with its ligand, for example by specifically binding to said ligand. “Specifically binding” means “specifically interacting with” whereby said interaction may be, inter alia, covalently, non-covalently and/or hydrophobic. Thus, an inhibitor which may be an antagonist may be a compound which inhibits or decreases e.g. the interaction between a protein and another molecule. Such inhibitors are described herein below in more detail or may be obtained by methods described herein. The inhibitors of the present invention preferably have a binding affinity to their corresponding ligand of at least 10⁵ M⁻¹, preferably higher than 10⁷ M⁻¹ and advantageously up to 10¹⁰ M⁻¹. Even higher binding affinities are not excluded from the invention.

It is envisaged that the inhibitors of the present invention are capable of a suppression or downregulation of the interferon γ signaling pathway i.e. they may directly interfere with the IFN-γ signaling pathway/cascade or may prevent the production/synthesis of a relevant gene product (target product) in a cell/subject in a way which is sufficient to suppress the interferon-γ signaling pathway/cascade to at least about 50% as compared to the natural state of said cell/subject. Preferably said inhibition efficiency is at least 60%, 70%, 80% or 90%. In a further preferred embodiment said inhibition rate is 100%. Said rate of inhibition may comprise but is not limited to suppression of the IFNγ signalling pathway, reduction of neointima formation in in-stent restenosis or a delayed time of lumen renarrowing after balloon angioblasty.

The term “plurality of compounds” is to be understood as a plurality of substances which may or may not be identical. The plurality of compounds may preferably act additively or synergistically. Said compound or plurality of compounds may be chemically synthesized or microbiologically produced and/or comprised in, for example, samples, e.g., cell extracts from, e.g., plants, animals or microorganisms. Furthermore, said compound(s) may be known in the art but hitherto not known to be capable of suppressing the INF-γ pathway.

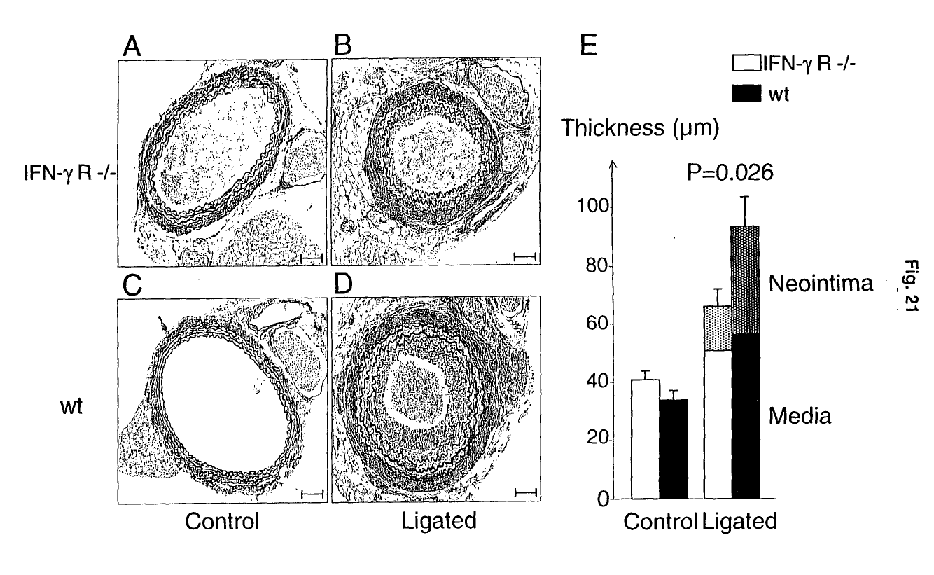

In accordance with the present invention it was surprisingly found that gene expression of components of the interferon-γ pathway and/or of gene expression of genes with described functions in the cellular response to interferon-γ (“target genes”) are upregulated in neointima, i.e. restenotic tissue. As shown in appended examples, it was, inter alia, suprisingly found that IRF-1, a pivotal transcription factor involved in IFN-γ signaling is overexpressed in smooth muscle cells (SMCs) of human neointima, i.e. in restenotically modified tissue. Furthermore, it could surprisingly be shown that in a mouse model (Huang 1993, Science 259, 1742-1745), deficient in the IFN-γ pathway (due to a knock-out of the corresponding IFN-γ receptor) restenotic and/or restenotic-like events could be reduced. In particular, the neointimal thickening was significantly reduced as shown in the appended examples and in FIG. 21.

IFN-γ is a cytokine, mainly produced by T-lymphocytes, which had previously been shown to be present in human atherosclerotic lesions (Ross, N. Engl. J. MEd. 340 (1999), 115-126). Thus, the presence of IFNγ in human atherosclerosis is well established (Ross, Am. Heart J. 138 (1999), S419-S420) but it is controversially discussed. Activation of T-cells which are present in atherosclerotic lesions at all stages of the process results in the secretion of IFNγ and TNF α and β (Ross, Am. Heart J. 138 (1999), S419-S420). In a xenotransplantation mouse models, IFNγ induces atherosclerotic changes in the absence of leukocytes in human arteries inserted into the aorta of immunodeficient mice by increasing the proliferative response of smooth muscle cells in cooperation with PDGF (Tellides, Nature 403 (2000), 207-211). In mouse models, the neutralization or genetic absence of IFNγ markedly reduced the extent of intimal expansion (Gupta, (1997) and Nagano, J. Clin. Invest 100 (1997), 550-557 and Nagano, Am. J. Pathol. 152 (1998), 1187-1197 and Raisanen-Sokolowski, Am. J. Pathol. 152 (1998), 359-365). In contrast other studies have shown that IFNγ inhibits proliferation of cultured smooth muscle cells (Bennett, Circ. Res. 74 (1994), 525-536 and Hansson, Circ. Res. 63 (1988), 712-719 and Hansson, J. Exp. Med. 170 (1989), 1595-1608 and Nunokawa, Biochem. Biophys. Res. Commun. 188 (1992), 409-415 and Shimokado, (1990) and Warner, J. Clin. Invest 83 (1989), 1174-1182) and matrix synthesis (Amento, Arterioscler. Thromb. 11 (1991), 1223-1230). This discrepancy may be due to a direct activation of macrophages by IFNγ or an indirect activation of T-cells by IFNγ via increased antigen presentation on macrophages.

However, as mentioned herein above, restenotic events and restenosis can clearly be distinguished from atheriosclerosis and the presence of T-lymphocytes in restenotic tissue(s) was never documented. Furthermore, whereas it was shown that IFN-γ inhibits proliferation and induces apoptosis in SMCs in vitro (Warner, J. Clin. Invest. 83 (1989), 1174-1182; Horiuchi, Hypertension 33 (1999), 162-166), absence of IFN-γ was documented to reduce intima hyperplasia in mouse models of atheroma and transplant arteriosclerosis (Gupta, J.Clin.lnvest. 99 (1995), 2752-2761; Raisanen-Sokolowski, Am. J. Pathol. 152 (1998), 359-365). Additionally, IFN-γ was shown to induce arteriosclerosis in absence of leukocytes in pig and human artery tissues inserted into the aorta of immunodeficient mice (Tellides, Nature, 403 (2000), 207-211). In summary, data obtained with in-vivo models of atherosclerosis argue in favor of a role for induction of atherosclerosis by IFN-γ, but an involvement of IFN-γor the IFN-γ induced signaling pathway had never been shown or even suggested in restenosis.

As illustrated in the appended examples, whereas previous studies identified CD45⁺ cells in restenotic tissue, said CD45⁺ cells have been classified as leukocytes. Furthermore, macrophages had been identified in said tissue (Kearney, Circulation 95 (1997), 1998-2002). The present invention surprisingly shows not only the presence of T-lymphocytes in restenotic tissue (employing a specific antibody directed against CD3), but, as mentioned herein above, an upregulation of genes involved in the IFN-γ signaling pathway and its target gene products.

In particular, as shown in the appended examples, T lymphocytes were identified in restenotic tissue by immunohistochemistry using an anti-human CD3 antibody. CD3-positive cells were detected in 3 out of 4 neointima samples, and a CD3-specific hybridization signal on cDNA arrays with 7 out of 10 neointima specimens from human patients could be detected (see appended examples). The surprising role for T-cells and/or IFN-γ in the development of neointima/restenotic tissue is here for the first time documented. The appended examples show an analysis of the gene expression pattern of human neointima from in-stent restenotic origin, which was removed during a surgical procedure using helix-cutter device atherectomy and a cluster of IFN-γ regulated genes upregulated in neonintima versus control tissue.

The pharmaceutical composition into which said inhibitor for the interferon γ-signaling pathway is formulated in accordance with the present invention may further comprise a pharmaceutically active acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 1 to 100 mg, preferably 10 to 50 mg, however, doses below or above these exemplary ranges are envisioned, especially considering the aforementioned factors. Generally, the regimen as a regular administration of the pharmaceutical composition should be in the range of 1 to 100 mg units, preferably 10 to 50 mg units per day. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously; or, e.g., by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Said pharmaceutical composition may further comprise drugs and/or prodrugs acting on smooth muscle cells, T-lymphocytes, macrophages or on myofibroblasts and/or being involved in blood pressure regulation, in blood coagulation or in inflammatory management.

Furthermore, the present invention relates to the use of an inhibitor for the IFN-γ signaling pathway for the treatment of restenosis, wherein said restenosis comprises restenosis of coronary arteries, carotid arteries, femoralis arteries or of other peripheral blood vessels, of aorta-coronary vein bypass, of arterial bypass, and/or of veinous bypass(es).

As mentioned herein above, a high rate of restenosis occurs after balloon angioplasty with subsequent stent implantation. Therefore, the present invention relates furthermore to the use of a an inhibitor for the interferon γ signaling pathway for the preparation of a pharmaceutical composition for the prevention of restenotic modifications before, during and/or after balloon angioplasty and/or stent implantation.

In a preferred embodiment of the use of the present invention said restenosis or restenotic modification is in-stent restenosis. The mechanism of restenosis after stent placement differs from that after PTCA: whereas the latter is due to remodeling with less neointimal proliferation, the contrary is true for in-stent restenosis; here, more than 90% of the late lumen loss is due to neointimal hyperplasia (Mintz, Am. J. Cardiol. 78 (1996), 18-22 and Hoffmann, Circulation 94 (1996), 1247-1254).

In a further preferred embodiment of the use of the present invention said inhibitor targets a gene and/or a gene product selected from the group consisting of IFN-γ, allograft inflammatory factor 1, APO-2 ligand (TRAIL), BCL-2 binding athanogene-1, C5a anaphylatoxin receptor, γ-interferon-inducible protein IP-30, interferon-γ receptor, interferon-γ receptor beta, interferon-induced 56 kDa protein, interferon-inducible protein 9-27, interferon regulatory factor-1, interferon regulatory factor-7, ISGF3-γ, lymphocyte antigen, p47-PHOX, pim-1 proto-oncogen, PYK2, and thrombospondin 1.

IFN-γ has been reported to induce proliferation as well as apoptosis. A key event in IFN-γ-induced growth inhibition and apoptosis is the induction of caspases (Dai, Blood 93 (1999), 3309-3316). Furthermore, IRF-1 induces expression of caspase-1 leading to apoptosis in vascular SMCs (Horiuchi, Hypertension 33 (1999), 162-166). As shown in the appended examples, an upregulation of the IFN-γ regulated genes for caspase-1, caspase-8 and DAP-1 could be documented in human neointima, i.e. restenotic tissue. A further gene known to be involved in pro-apototic function is APO-2 ligand (TRAIL), a target gene product of the IFN-γ signaling pathway. As shown in the appended examples, the (coding) message encoding for APO-2 ligand (TRAIL) was also upregulated in human neointima/restenotic tissue. However, mRNAs for the anti-apoptotic proteins BAG-1, Pim-1 (both regulated by IFN-γ) and BCL-2-related protein A1 were also upregulated in neointima versus control, supporting the notion that proliferation and apoptosis occur simultaneously in human neointima with a preponderance of proliferation. Furthermore, the appended examples document that incubation of proliferating smooth muscle cells with IFN-γ resulted in an adaptation of the expression pattern to that of neointima from patients, without inducing apoptosis in the smooth muscle cells.

As already mentioned before, an involvement of IFNγ in pathogenesis of restenosis was not described up to now. In the appended examples a proinflammatory expression pattern was observed which is characterized by the presence of markers for macrophages and T-lymphocytes and by the expression of numerous genes involved in the cellular response to IFNγ. IFNγ reduces the basal rate of apoptosis in proliferating smooth muscle cells and attenuates hydrogen peroxide-induced apoptosis (see appended examples). Also in vivo data which are described hereinafter in the appended examples obtained in a mouse model of restenosis using mice lacking the IFNγ receptor demonstrated a significantly reduced vascular proliferative response.

Besides the above mentioned upregulation of the transcription factor IRF-1, it has been surprisingly found that another transcription factor involved in IGF-γ signal is upregulated in restenotic tissue. Furthermore, an upregulation of the tyrosine kinase Pyk2 has been observed. Interestingly, it had been previously found that Pyk2 is activated by IFN-γ and inhibition of Pyk2 in NIH3T3 cells resulted in a strong inhibition of the IFN-γ induced activation of MAPK and STAT1 (Takaoka, EMBO J. 18 (1999), 2480-2488).

In addition, the appended examples show that, regarding the IFN-γ pathway, not only the genes for the complete IFN-γ receptor, the main transcription factors, components of the signal transduction pathway (Dap-1, BAG-1, Pim-1, IFN-γ-inducible protein, IFN-inducible protein 9-27) were induced in restenotic tissue but also several target genes of the IFN-γ pathway, like CD40, CD13 and thrombospondin-1, as well as caspase-1, caspase-8, ICAM-1, integrin beta 7 or p47-PHOX were upregulated.

In an additional preferred embodiment of the use of the present invention said inhibitor is an antibody or a functional derivative or functional fragment thereof, an aptamer, a riboyzyme, an antisense-oligonucleotide, a DNA binding protein, a peptide, protein or histamine.

The term “functional derivative or functional fragment” in connection with antibody is defined as follows: Functional antibody, fragments or derivatives comprise Fab fragments, F(ab′)₂, Fv or scFv fragments; see, for example, Harlow and Lane, “Antibodies, A Laboratory Manual”, CSH Press, Cold Spring Harbor, 1988.

Said antibody (or an derivative or fragment thereof), aptamer, riboyzyme, antisense-oligonucleotide, DNA binding protein, peptide, protein or histamine may directly interfere with the IFN-γ signaling pathway/cascade or may prevent the production/synthesis of a relevant gene product (target product). For example, it is envisaged that an antibody (or derivative or fragment thereof) may interfere with the receptor-ligand binding of IFN-γ to its receptor. Said antibody (or derivative or fragment thereof) may be, inter alia, directed against IFN-γ itself or the (preferably) extracellular part of the IFN-γ receptor α and/or β chain).

However, said inhibition also comprises the interference with gene expression of relevant gene products of the IFN-γ signaling pathway and/or-with the gene expression of its target gene products. Said interference may comprise, inter alia, the use of aptamers, ribozymes, antisense-oligonucleotides or DNA-binding proteins. Furthermore, specific peptides and/or polypeptides/proteins may be employed for said inhibition. These peptides/proteins may be, inter alia, involved in the inhibition of relevant signaling cascades, like phosphorylation cascades and may serve as inhibiting substrates of said phosphorylation events. In addition, said peptides/proteins may interfere with said IFN-γ signaling pathway via protein-protein interaction(s).

The above mentioned antibody may be a monoclonal or a polyclonal antibody. Techniques for the production of antibodies are well known in the are and described, e.g. in Harlow and Lane, loc. cit.. The term “antibody” comprises chimeric, single chain and humanized antibodies. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies; Also, transgenic animals may be used to express, e.g. humanized antibodies useful in this invention. Most preferably, the antibody to be used in accordance with the invention is a monoclonal antibody. The general methodology for producing, monoclonal antibodies is well-known and has been described in, for example, Kohler and Milstein, Nature 256 (1975), 495-497 and reviewed in J. G. R. Hurrel, ed., “Monoclonal Hybridoma Antibodies: Techniques and Applications”, CRC Press Inc., Boco Raron, Fla. (1982), as well as that taught by L. T. Mimms et al., Virology 176 (1990), 604-619.

The antibodies which may function, in accordance with this invention, as an inhibitor of the IFN-γ signaling pathway may be an antibody directed against any component of said pathway or any target gene product of said pathway as described herein above. Particularly preferred are antibodies, fragments or derivatives thereof which are directed against IFN-γ, allograft inflammatory factor 1, APO-2 ligand (TRAIL), BCL-2 binding athanogene-1, C5a anaphylatoxin receptor, γ-interferon-inducible protein IP-30, interferon-γ receptor, interferon-γ receptor beta, interferon-induced 56 kDa protein, interferon-inducible protein 9-27, interferon regulatory factor-1, interferon regulatory factor-7, ISGF3-γ, lymphocyte antigen, p47-PHOX, pim-1 proto-oncogen, PYK2, and thrombospondin 1.

The genes and/or gene products (transcripts) encoding for the above mentioned components of the IFN/IFN-γ signal transduction pathway or for IFN-γ itself may also be targeted by (an) aptamer(s). Said aptamer(s), therefore, may also function as an inhibitor for the IFN-γ signaling pathway.

In accordance with the present invention, the term aptamer means nucleic acid molecules that can bind to target molecules. Aptamers commonly comprise RNA, single stranded DNA, modified RNA or modified DNA molecules. The preparation of aptamers is well known in the art and may involve, inter alia, the use of combinatorial RNA libraries to identify binding sides (Gold, Ann. Rev. Biochem. 64 (1995), 763-797). Similarly, ribozymes may function as inhibitor for the IFN-γ signaling pathway, inter alia, by interfering with relevant mRNA molecules. In the context of the present invention ribozymes comprise, inter alia, hammerhead ribozymes, hammerhead ribozymes with altered core sequences, deoxyribozymes (see, e.g., Santoro, Proc. Natl. Acad. Sci. USA 94 (1997), 4262) and may comprise natural and in vitro selected and/or synthesized ribozymes. Constructions of ribozymes are also described in, e.g., EP-B1 0 291 533, EP-A1 0 321 201, EP-A2 0 360 257 which specifically cleave RNA encoding for components of the INF-γ signaling pathway or its target gene products. Selection of appropriate target sites and corresponding ribozymes can be done as described for example in Steinecke, Ribozymes, Methods in Cell Biology 50, Galbraith, eds. Academic Press, Inc. (1995), 449-460.

Furthermore, a polynucleotide/oligonucleotide complementary to a RNA encoding for components of the IFN-γ signaling pathway may be used for the construction of appropriate anti-sense oligonucleotides which are able to inhibit the IFN-γ signaling pathway. Said antisense-oligonucleotide comprises preferably at least 15 nucleotides, more preferably at least 20 nucleotides, even more preferably 30 nucleotides and most preferably at least 40 nucleotides. Therefore, the present invention relates furthermore to the use of an anti-sense oligonucleotide which specifically inhibits the function of RNAs encoding gene products involved in the IFN-γ signaling pathway or encoding its target genes.

As mentioned herein above and in accordance with the present invention (a) peptide(s) or protein(s) may be used as inhibitors for the IFN-γ signaling pathway. It is preferred that said peptide(s) or protein(s) are recombinantly produced. The recombinant production of said peptide(s) or protein(s) is well known in the art (see, inter alia, Sambrook (Molecular Cloning; A Laboratory Manual, 2^{nd} Edition, Cold Spring Harbour Laboratory Press, Cold Spring Harbour, N.Y. (1989)).

In addition, histamine can be employed as an inhibitor of the IFN-γ signaling pathway, since histamine had been shown to reduce gene expression and biosynthesis of IFN-γ (Horvath, Immunol. Lett. 70 (1999), 95-99).

In a further preferred embodiment of the use of the invention said inhibitor is selected form the group consisting of inhibitors of IFN-γ production, APO-2 ligand (TRAIL) transcription, inhibitors of C5a anaphylatoxin receptor, inhibitors of p47-phox, inhibitors of PYK2 and inhibitors of thrombospondin-1.

Known inhibitors of IFN-γ production are calmodulin antagonists like N-(6-aminoehexyl)-5-chloro-1-naphtalenesulfonamide (W-7) (Antonelli, Interferon Res. 8 (1988), 193-200) or inhibitors of histamine (Carlson, Cell Immunol. 96 (1985), 104-112). Inhibitors of TRAIL transcription are herbimycin, genistein, PKC inhibitors like staurospprin, P13-K inhibitors like wortmannin, cyclosporin A and rapamycin (Musgrave, Exp. Cell Res. 252 (1999), 96-103). Inhibitors of C5a anaphylatoxin receptor are also known to the skilled artisan and comprise semi-synthetic or synthetic C5a anaphylatoxin receptor antagonists and/or inhibiting antibodies of C5a anaphylatoxin receptor, cyclic antagonists F-(OpdChaWr), an acyclic derivative thereof, MeFKPdChaWr or the use of (a) specific peptide(s) to antagonize C5a anaphylatoxin receptor as described in Kaneko, Immunology 86 (1995), 149-154; Baranyi, J. Immunol. 157 (1996), 4591-4601; Pellas, Curr. Pharm. Des. 5 (1999), 737-755; Haviland, J. Immunol. 154 (1995), 1861-1869; Mizuno, Clin. Exp. Immunol. 119 (2000), 368-375; Short, Br. J. Pharmacol. 126 (1999), 551-554; Wetsel, Immunol. Left. 44 (1995), 183-187; Finch, J. Med. Chem. 42 (1999), 1965-1974 or Pellas, J. Immunol. 160 (1998), 5616-5621. Furthermore inibitors of p47-Phox comprise protein kinase C inhibitor, GF 109203X (Yaname, Arch. Biochem. Biophys. 361 (1999), 1-6), serine protease inhibitor like 4-(2-aminoethyl)-benzenesulfonyl fluoride (AEBSF) (Diatchuk, J. Biol. Chem. 272 (1997), 13292-13301), or cAMP agonists (Bengis-Garber, Cell Signal 8 (1996), 291-296). Inhibitors of PYK2 are known inhibitors of serine/threonine kinases or inhibitors of protein kinase C, inhibitors of MEK or the transcriptional inhibitor actinomycin D (to block PYK2 expression) (Tsuchida, J. Immunol. 163 (1999), 6640-6650). An inhibitor to the extracellular matrix protein thrombospondin-1 comprises, inter alia, an inhibiting antibody to thrombospondin-1 as described in Chen, Circulation 100 (1999), 849-854.

Another object of the present invention concerns the identification of drugs and/or prodrugs for subject/patients suffering from restenosis or being prone to suffer from restenosis. Therefore, in yet another embodiment the present invention relates to a method for the identification of an inhibitor for the IFN-γ signaling pathway, comprising the steps of:

a. culturing a cell line in the presence of IFN-γ or a functional derivative thereof and in the presence of a (test) compound or a sample comprising a plurality of (test) compounds under conditions which permit IFN-γ signaling; and

b. detecting and/or verifying a (test) compound or a sample comprising a plurality of (test) compounds, which is capable of a suppression of said IFN-γ signaling pathway.

The term “functional derivative” of IFN-γ relates to derivatives that retain or essentially retain the biological properties of natural IFN-γ. Examples of such derivatives are muteins. The same applies, mutatis mutandis, for other components mentioned herein.

Thus, the findings of the present invention provide the options of indentification of further inhibitors for the IFN-γ signaling pathway (or the functional assessment of known inhibitors for the IFN-γ signaling pathway) which may be employed in the use of the present invention and/or in the methods preventing and/or treating restenosis as described herein below. The above described method is particularly useful for testing the capacity of (test) compounds (or samples comprising a plurality of (test) compounds) as drugs and/or prodrugs for the prevention and/or treatment of restenosis.

In accordance with the present invention, cell line(s) which may be employed in the above described method are known to the person skilled in the art and comprise, but are not limited to, cultured smooth muscle cells (preferably coronary artery smooth muscle cells, CASMCS), smooth muscle cells endothelial cells, fibroblasts, monocytes, macrophages and T-lymphocytes. Said cells are easily obtainable for the person skilled in the art, e.g. from relevant depositaries like DSMZ, Braunschweig or the ATCC.

The term “compound” or “(test) compound” in the method of the invention includes a single substance or a plurality of substances which may or may not be identical.

The “presence” of said (test) compound may involve co-culturing with IFN-γ or may involve contacting said cell line with said (test) substance before, during and/or after IFN-γ was added to the culture. Said (test) compound(s) may be comprised in, for example, samples, e.g., cell extracts from, e.g., plants, animals or microorganisms. Furthermore, said compounds may be known in the art but hitherto not known to be capable of inhibiting the IFN-γ signaling pathway or not known to be useful as an inhibitor of said pathway, respectively. The plurality of compounds may be, e.g., added to the culture medium or injected into the cell.

If a sample containing (a) compound(s) is identified in the method of the invention, then it is either possible to isolate the compound from the original sample identified as containing the compound in question, or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein and in the appended examples. Depending on the complexity of the samples, the steps described above can be performed several times, preferably until the sample identified according to the method of the invention only comprises a limited number of or only one substance(s). Preferably said sample comprises substances or similar chemical and/or physical properties, and most preferably said substances are identical. The methods of the present invention can be easily performed and designed by the person skilled in the art, for example, in accordance with other cell based assays described in the prior art or by using and modifying the methods as described in the appended examples. Furthermore, the person skilled in the art will readily recognize which further compounds and/or cells may be used in order to perform the methods of the invention. Such adaptation of the method of the invention is well within the skill of the person skilled in the art and can be performed without undue experimentation.

The above mentioned detection and/or verification step in the method for the identification of an inhibitor for the IFN-γ signaling pathway may, inter alia, involve the measurement of the quantity of expressed gene products (for example the expression/expression level of target genes known to be regulated by IFN-γ) as well as the measurement of specific phosphorylation events occurring in IFN-γ signaling. Said methods of detection and/or verification are well known in the art and comprise, inter alia, in vivo phosphorylation assays (P³² labelings) ELISAs, Northern-, Southern-, and/or Western-blotting, 2D-gel electrophoresis as well as proteome analysis. Furthermore, IFN-γ activity may be measured as described, inter alia, in Meager, “Lymphokines and Interferons” (1987).

As known to the person skilled in the art, analysis of proteomes of lower complexity, e.g. ribosomes with 60 protein species, can be performed, inter alia, by protein/protein species separation and identification strategies, comprising, for example, 2-dimensional gel electrophoresis (2-DE; Kaltschmidt, Anal. Biochem. 36 (1970), 401) or HPLC (Kamp, J. Chromatogr. 317 (1984), 181). Analysis of proteomes of higher complexity can be carried out, inter alia, by a combination of isoelectric focusing and SDS-PAGE (Vesterburg, Acta Chem. Scand. 20 (1966), 820; Laemmli, Nature 227 (1970), 680) and the use of large-sized gels (Jungblut, Electrophoresis 15 (1994), 685; Klose, Electrophoresis 16 (1995), 1034). Comparison of individual, specific 2-DE gels allows for the identification of differentially expressed proteins and the identification of proteins separated by 2-DE is known to the skilled artisan (see, e.g. Patterson, Electrophoresis 16 (1995), 1791; Jungblut, Electrophoresis 17 (1996), 839; Jungblut, Mass Spectrometry Reviews 16 (1997), 145).

Further methods comprise protein elution, MALDI-MS, N-terminal sequencing by Edman degradation (Edman, Acta Chem. Scand. 4 (1950), 283), enzymatic in-gel digestion, analysis of peptides directly in the mixture by mass spectrometry, peptide mass fingerprinting (Pappin, Curr. Biol. 3, (1993), 327), PSD-MALDI-MS (Spengler, Rapid Commun. Mass Spectrom. 6, (1992), 105), ESI-MS (electrospray-ionization-MS) and/or (after separation) by micro-HPLC. HPLC separated peptides may be further analysed, inter alia, by Edman degradation, PSD-MALDI-MS, MS/MS (Wilm, Nature 379, (1996), 466) or ladder sequencing (Thiede, FEBS Lett. 357, (1995), 65). Proteins/peptides immobilized on membranes allow the identification and/or quantification by immunostaining (Towbin, Proc. Natl. Acad. Sci. USA 76, (1979), 4350).

In a further embodiment of the present invention said detection and/or verification step comprises a transcriptome analysis. Said analysis may be carried out by methods known in the art, inter alia, by analysis of mRNA transcripts on cDNA arrays (as described, inter alia, in Bryant, PNAS U.S.A. 96 (1999), 5559-5564 or Mahadevappa, Nat. Biotech. 17 (1999), 1134-1136). However, methods for global amplification of polyadenylated mRNA as shown in the appended examples may also be employed in order to elucidate what kind of transcripts are up- or downregulated due to the presence of said (test) compound(s) and IFN-γ during culturing of said cell line.

Furthermore, the present invention relates to a method for preventing and/or treating restenosis in a subject comprising the step of administering to a subject suffering from said restenosis and/or to a subject susceptible to suffer from said restenosis an effective amount of an inhibitor for the interferon γ signaling pathway as defined herein.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

In addition, the present invention relates to a method for prevention of restenotic modification before, during and/or after balloon angioplasty and/or stent implantation in a subject comprising the step of administering to said subject an effective amount of an inhibitor for the interferon γ signaling pathway as defined herein above, during and/or after balloon angioplasty and/or stent implantation.

It is particularly preferred that the subject to be treated is a human and/or that said prevention of restenosis/restenotic modification is in a human subject. Furthermore, the embodiments as described herein above for the use of the present invention also apply, mutatis mutandis, for the method for preventing and/or treating restenosis or for prevention restenotic modification before, during and/or after balloon angioplasty and/or stent implantation as described herein above.

The Figures show:

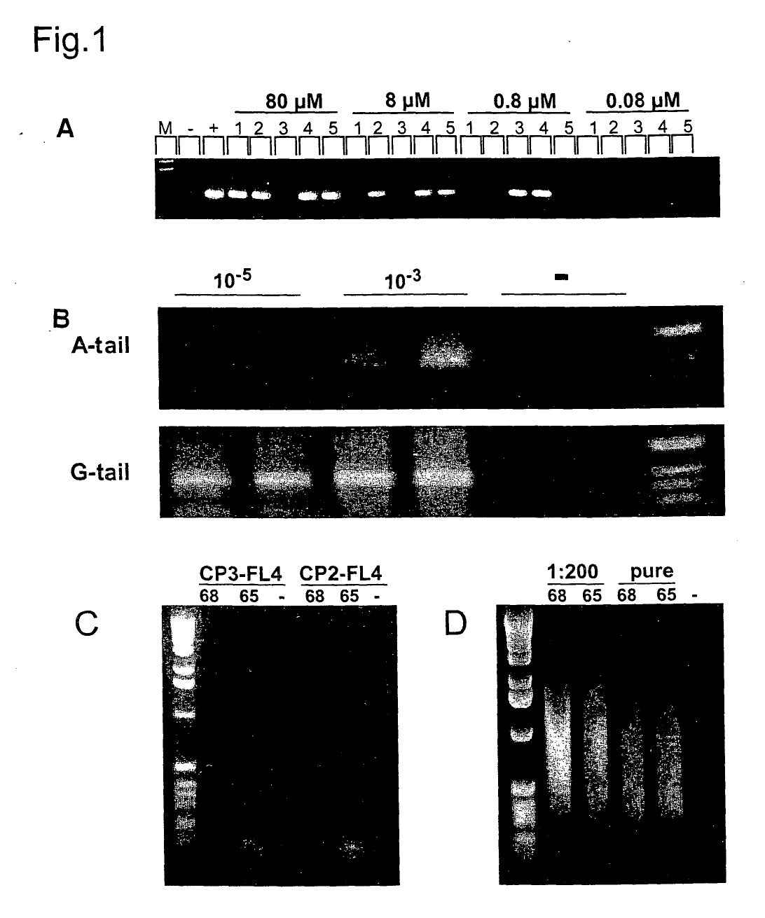

FIG. 1. Parameters determining amplification success. a) Twenty HT29 colon carcinoma cells ((ATCC: HTB-38) lanes 1-20) were individually isolated and mRNA reverse transcribed in the presence of different concentrations of random hexamer primers (lanes 1-5, 80 μM; lanes 6-10, 8 μM; lanes 11-15, 0.8 μM; lanes 16-20, 0.08 μM). 1/10 of the cDNA was subsequently tested for the detection of the ki-ras transcript by gene-specific PCR. b) Influence of the homopolymer tail on sensitivity. A 350 bp TGF-α fragment was isolated, diluted and either dA or dG tailed. Serial dilutions were tested by PCR using poly-dT or poly-dC containing primers, respectively, and a primer within the TGF-α sequence. The informative dilutions are shown in duplicates. (lanes 1+2, negative control; lanes 3+4, 10⁻³ dilution; lanes 5+6, 10⁻⁵ dilution). c) FL4-N6 primed and revers transcribed mRNA was dG-tailed and amplified using the CP3+FL4 primers (lanes 1-3) or CP2+FL4 primers at different annealing temperatures (lane 1+4, 68° C., lane 2+5 65° C., lane 3+6, negative control). d) An identical amount of mRNA as in c) was reverse transcribed using the CFL5cN6 primer, and amplified with the CP2 primer. An equal amount of cDNA as in c) (lane 3+4) resulted in amplification of a wide range of cDNA fragments as did a 1:200 dilution (lane 1+2) at different annealing temperatures (lane 1+3, 68° C.; lane 2+4, 65° C.; lane 5, negative control).

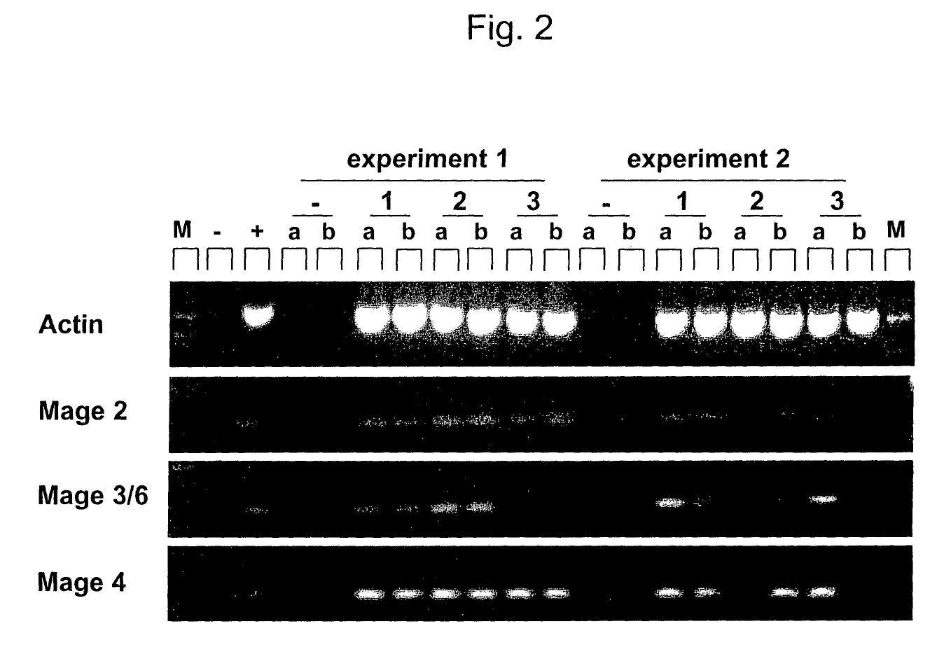

FIG. 2. Gene specific PCR for β-actin and various MAGE transcripts using unamplified pooled cDNA of A431 cells as positive control (+) and amplificates of single A431 cells (lane 2-4 and 6-8) that were divided into two halves (a+b) before global PCR. Two independent experiments were performed (lane 1-4 and 5-8) with lane 1 and lane 5 being the negative controls for the global PCR.

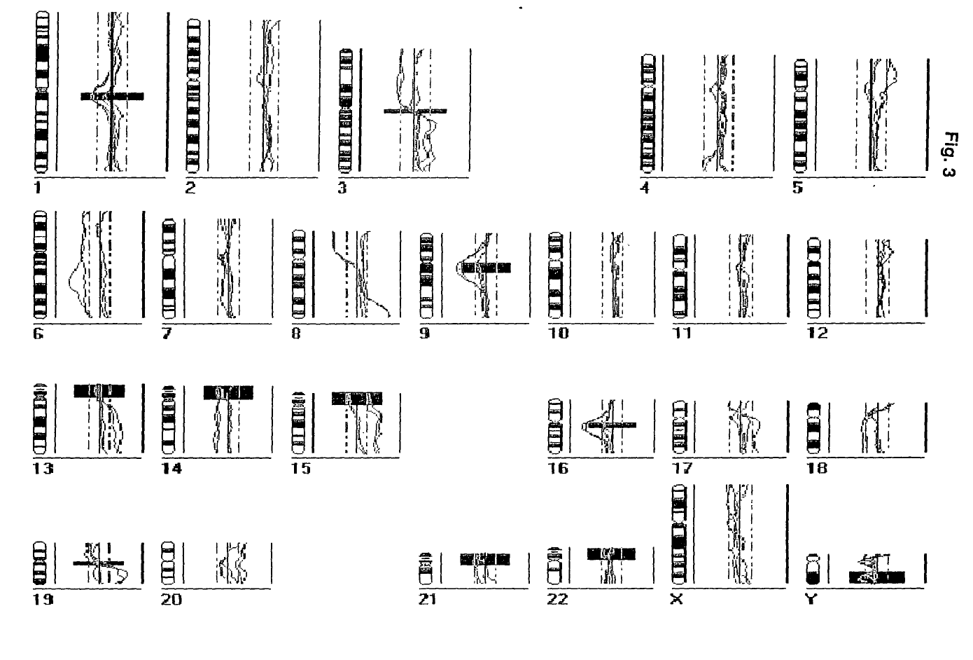

FIG. 3. CGH profiles of two normal leukocytes (red) and two MCF-7 breast cancer cells (blue) of which the genomic DNA was isolated from the supernatant after mRNA isolation. The chromosomal ratios of the normal cells are within the dashed lines, giving the threshold for significance, whereas the profiles of the cancer cells are similar with regard to their chromosomal deletions and amplifications.

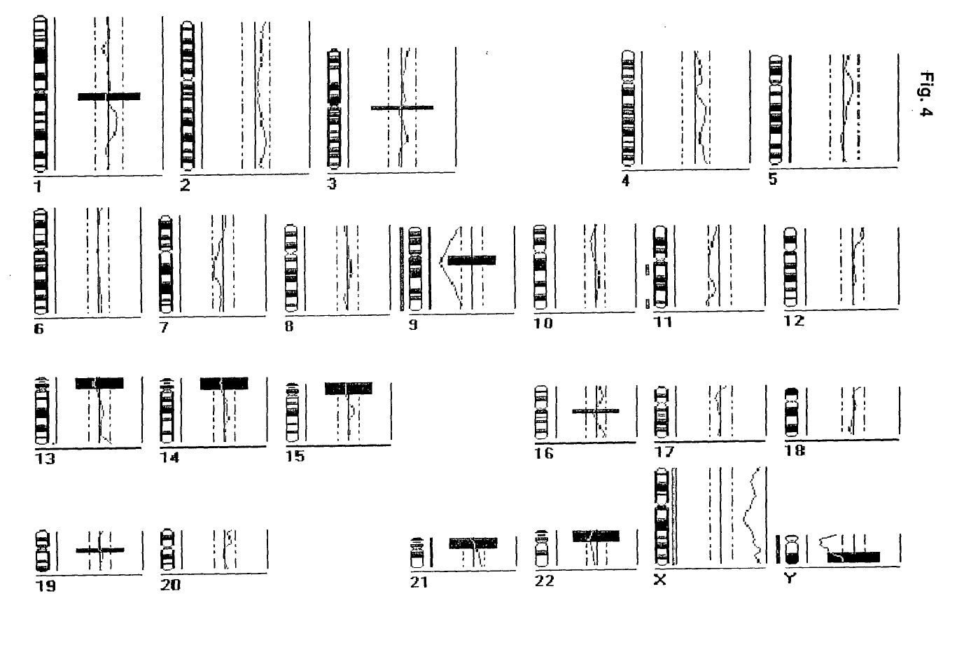

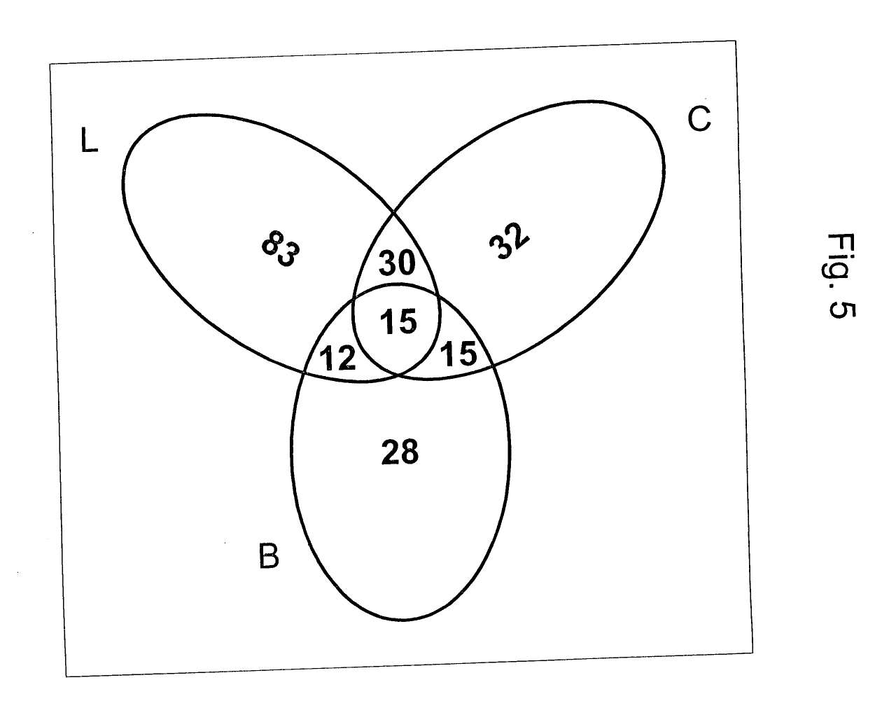

FIG. 4. CGH profile of cell B derived from a breast cancer patient with very small primary tumor (stage T1a). Chromosomal deletions are marked with a red bar left of and chromosomal gains with a green bar right of the chromosome symbol.

FIG. 5. Diagram illustrating the common and differentially expressed genes of cell B, C and L.

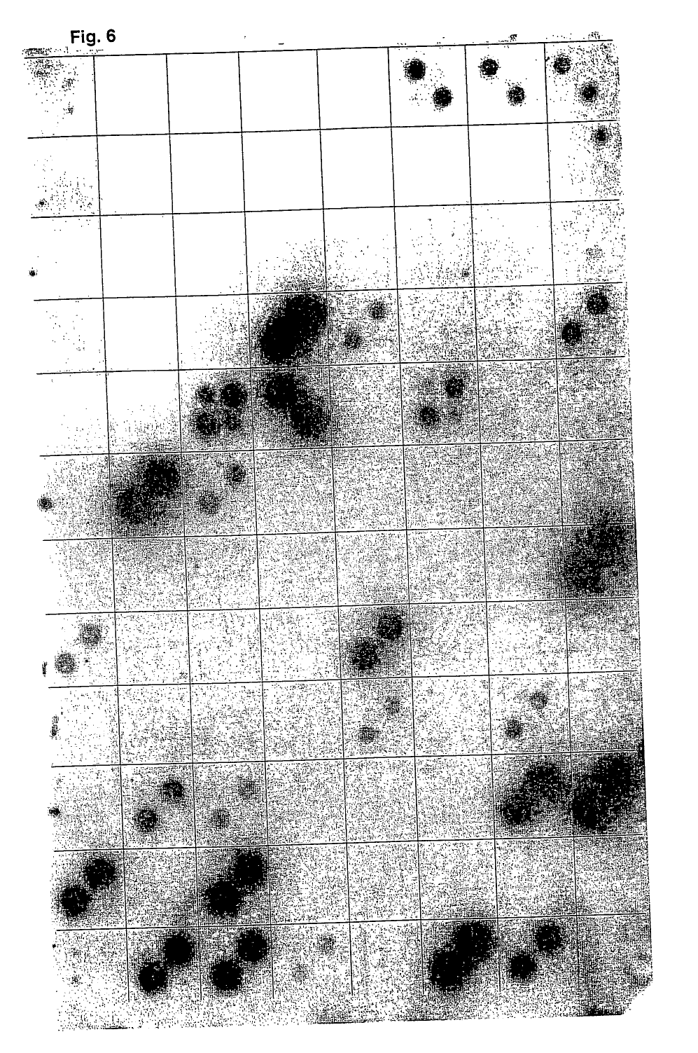

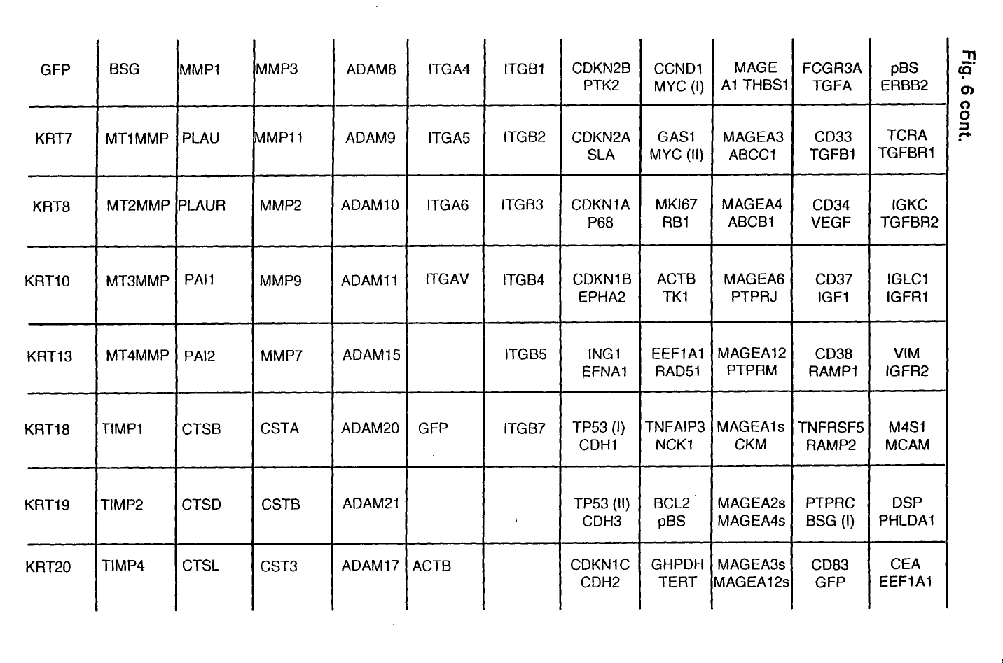

FIG. 6. Hybridisation of cell L (left) and the matrix of positions and names of immobilized cDNAs. Genes were spotted in duplicates in diagonal direction, with the blue gene symbols oriented from upper left to lower right and the red gene symbols oriented from upper right to lower left.

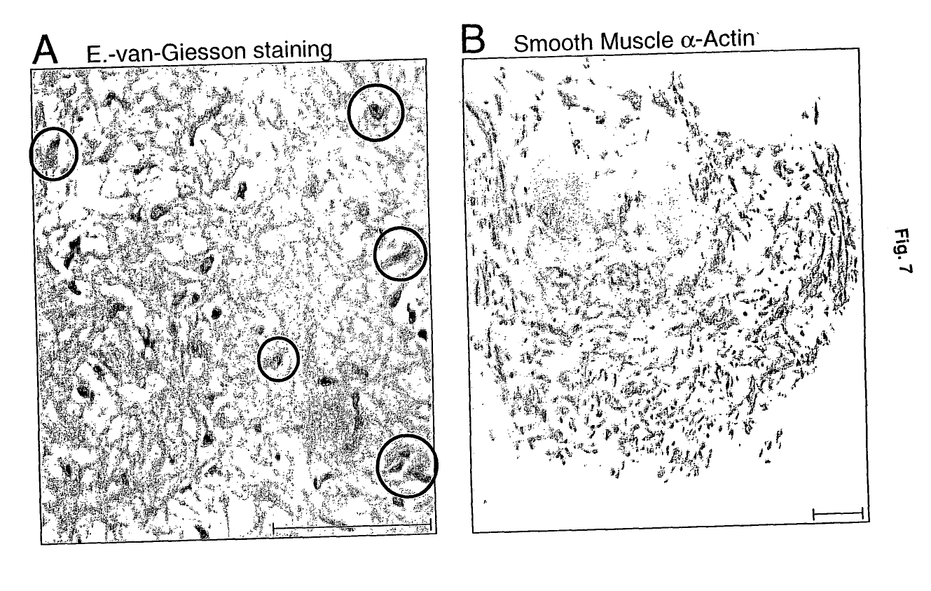

FIG. 7: Immunohistochemical stains of neointima from human coronary artery in-stent restenosis for v. Giesson (left panel) and smooth muscle alpha-actin (right panel). The shown experiment is a representative of 3 independent specimen. Bars indicate 100 μm.

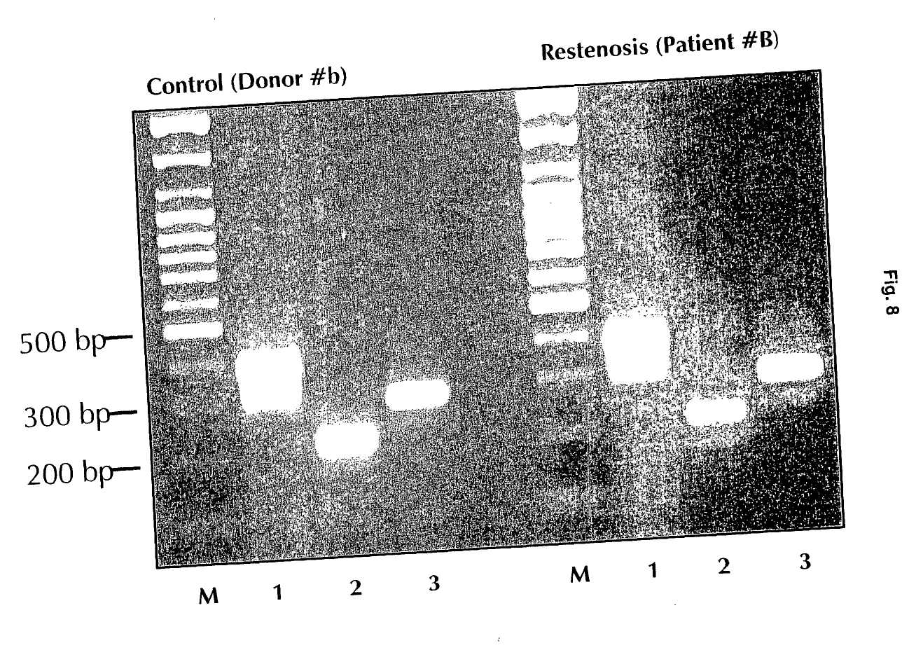

FIG. 8: PCR with gene-specific primer for β-actin (lanes 1), EF-1α (lanes 2) and α-actin (lanes 3) as a control for successful PCR amplification of the first strand cDNA generated from microscopic tissue specimen. Shown is one representative from each study group (right panel: patient B; left panel: control donor b). The position of three size markers (M) is shown.

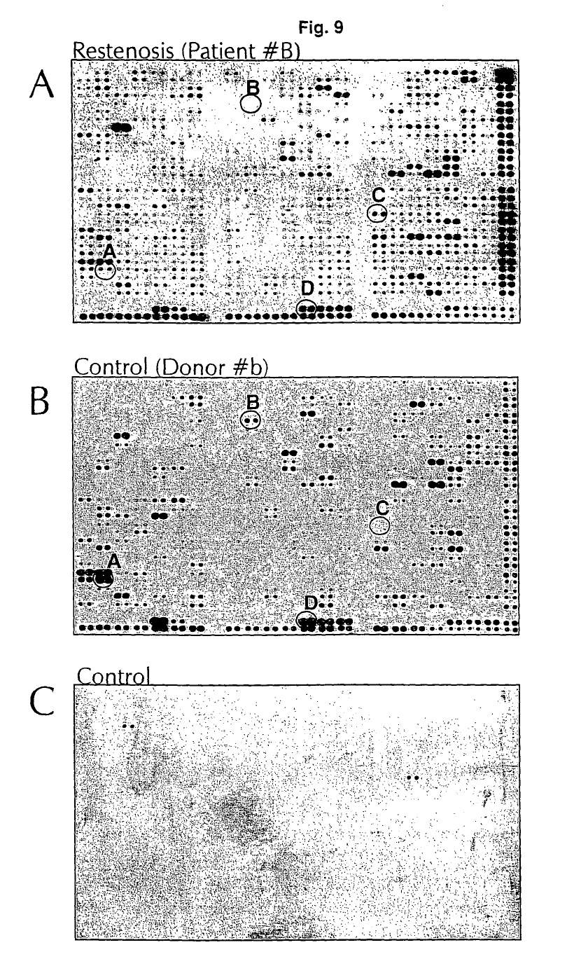

FIG. 9: cDNA array analysis. The same array is shown with three independent hybridization experiments comparing mRNA isolated from neointima (panel A) or from control vessel (panel B), and in the absence of a biological sample (panel C). The cDNA array contained 588 genes including nine housekeeping genes and three negative controls [M13 mp 18 (+) strand DNA; lambda DNA; pUC18 DNA]. The experiment shown here is a representative of hybridization experiments with 10 neointima and 10 control specimen. Circles indicate four hybridization signals (A-D) differentially expressed between restenosis and control.

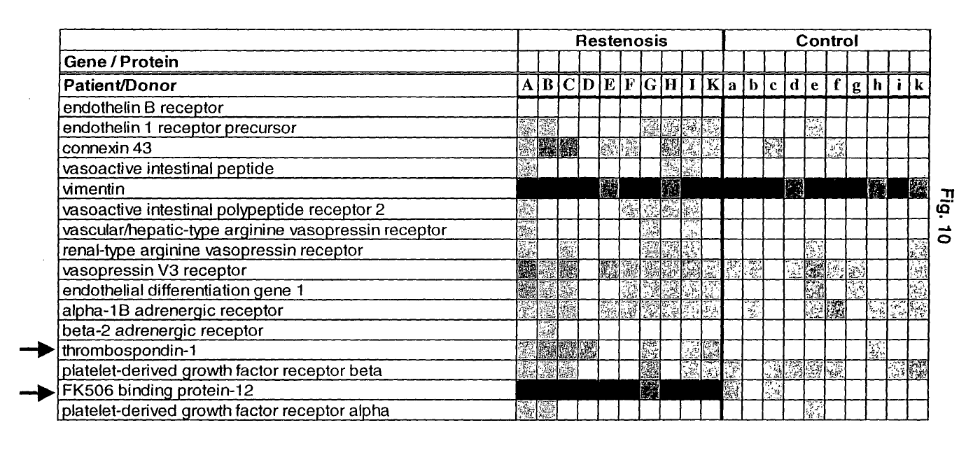

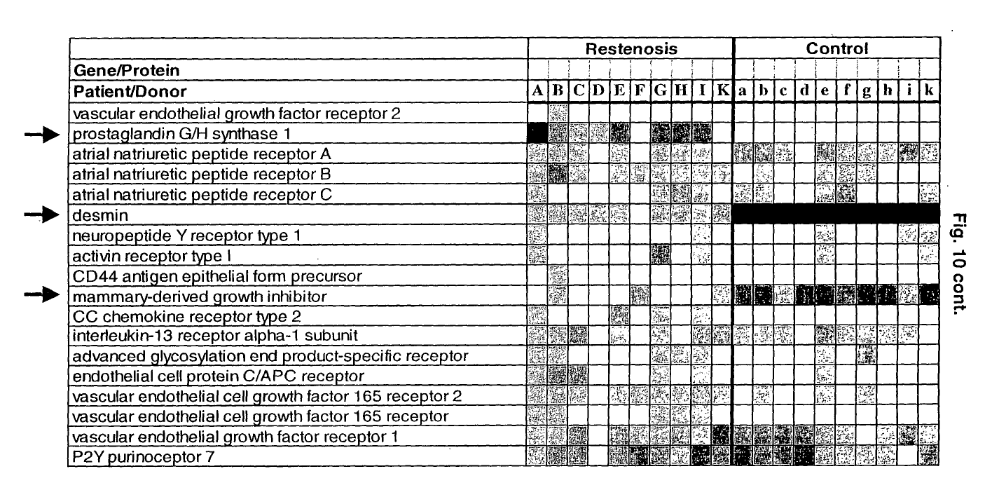

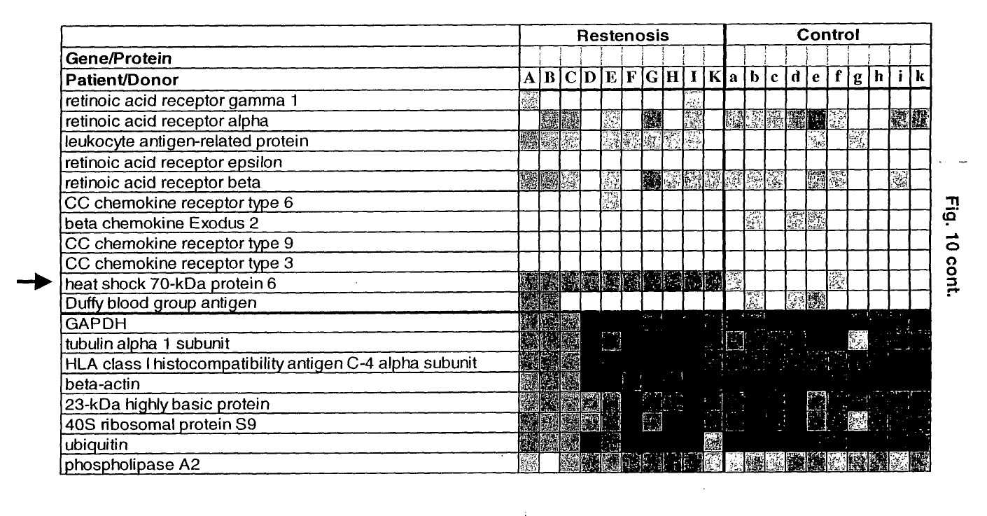

FIG. 10: Transcription profiles of microscopic samples from human in-stent neointima and control vessels. Each column represents a gene expression analysis of a single specimen for 53 selected genes. An arrow indicated genes that show significant up- or downregulation in neointima versus control. Eight highly expressed housekeeping genes are shown on the bottom. One grey value corresponds to a signal intensity as shown at the bottom of the figure.

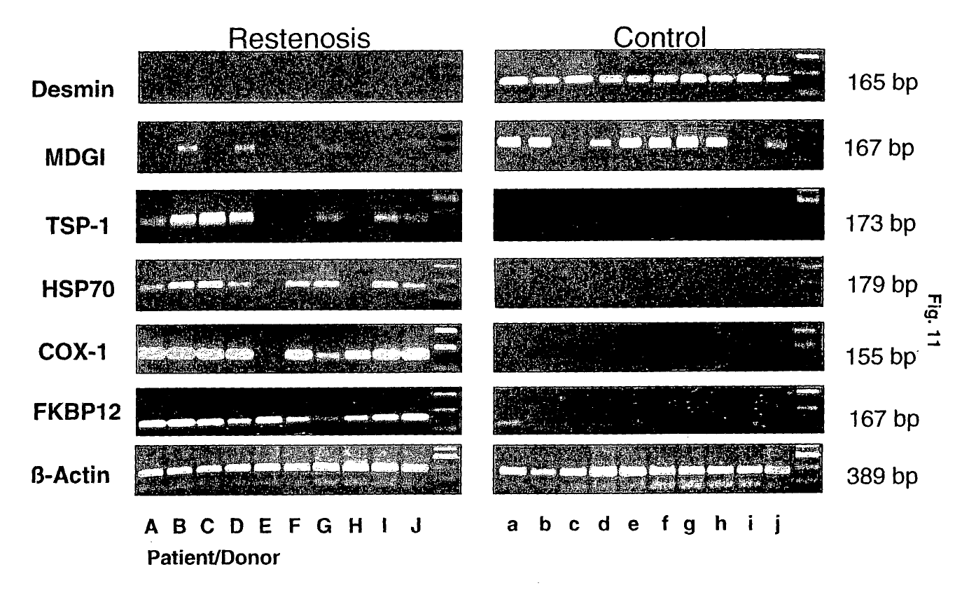

FIG. 11: Verification of differentially expressed mRNAs from cDNA arrays by gene-specific PCR. The size of the expected PCR fragment is indicated on the right.

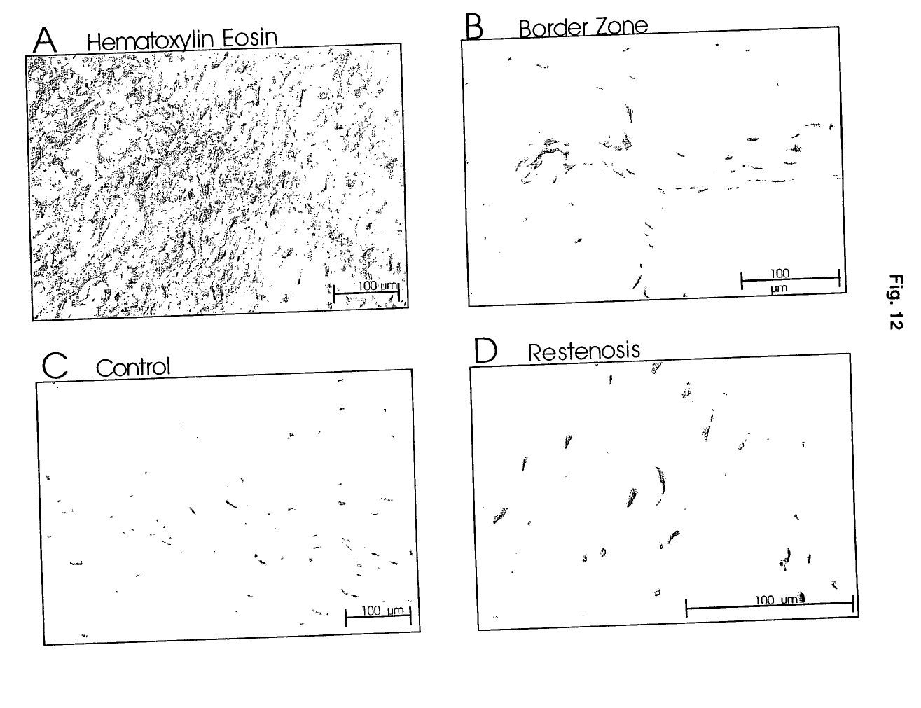

FIG. 12: Immunhistochemical staining of neointima from carotid artery restenosis for the FKBP12 protein. The experiment shown is a representative of three independent experiments. The bars represents a distance of 100 μm. Panel A shows a hematoxylin eosin staining, panel B-D shows staining for FKBP12 of the border zone between healthy media and neointima (panel B), of healthy control media (panel C) and neointimal tissue (panel D).

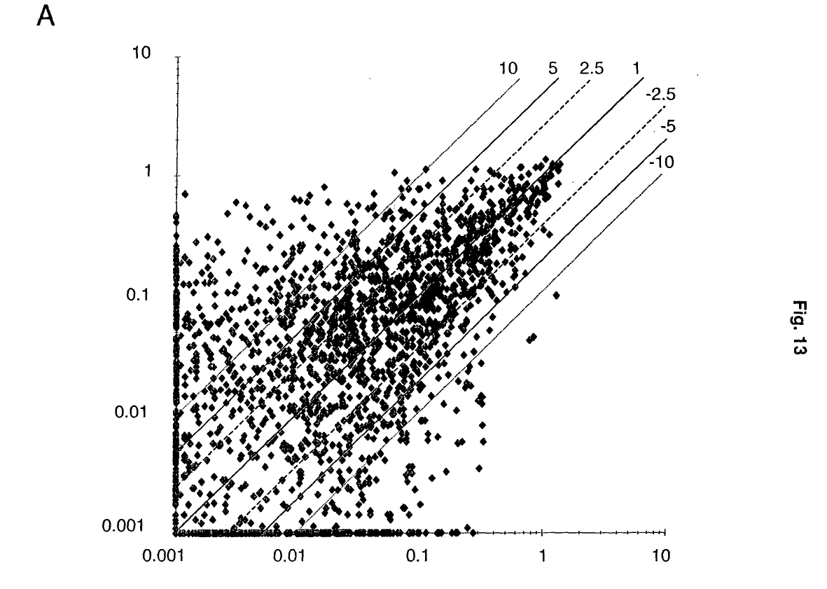

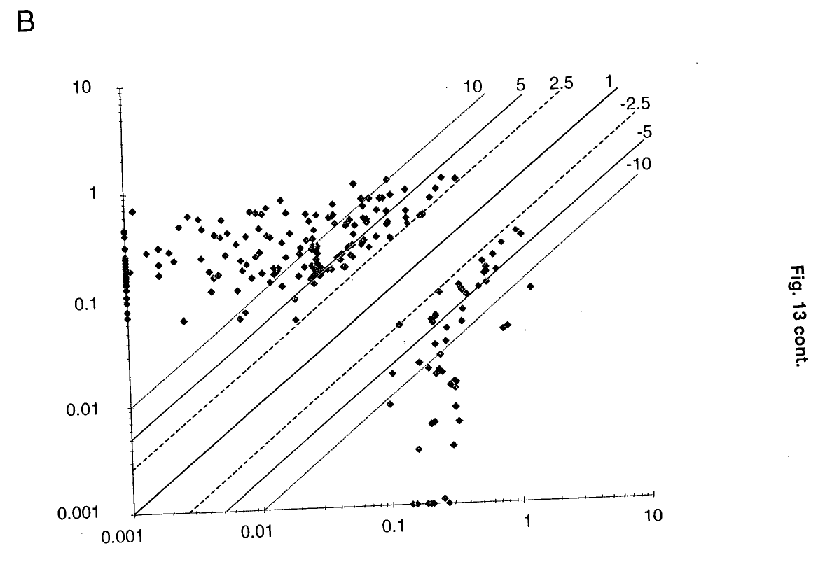

FIG. 13: cDNA array analysis of gene expression. Four Clontech Atlas microarrays, containing a total of 2435 human cDNAs, were hybridized with cDNA labeled with Dig-dUPT prepared from RNA from in-stent neointima (n=10) and from control media/intima (n=11) as described in Materials and Methods. Spots indicate the mean of the relative expression of the two examined groups. Panel A shows the expression of all examined genes in this study. Panel B shows expression of the 224 differentially expressed genes, that were more than 2.5-fold induced or reduced in neointima and showed a statistical significance p<0.03 in the Wilcoxon test. For this presentation, zero value were replaced by a value of 0.0001, as a zero value is not representable in a logarithmic scale.

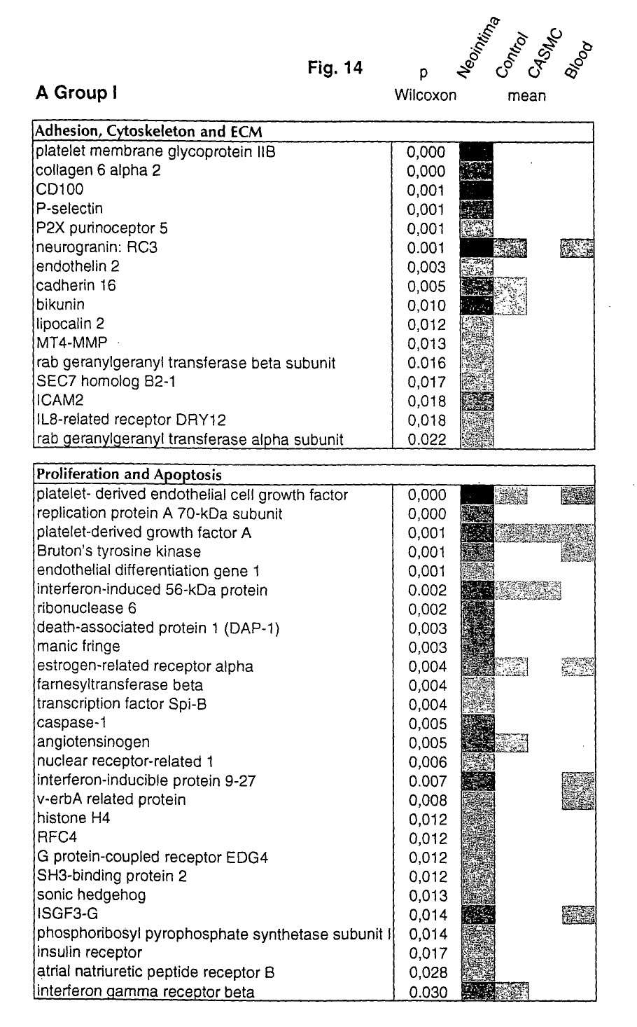

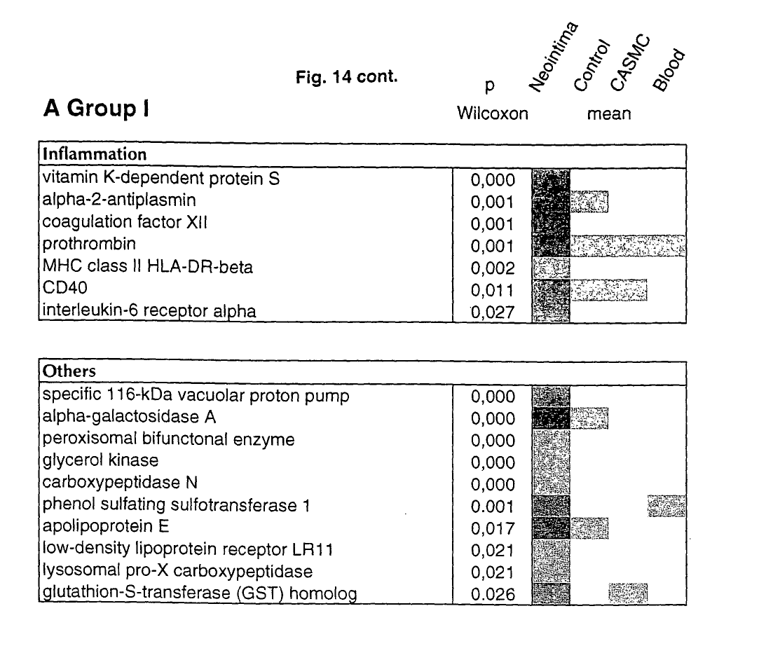

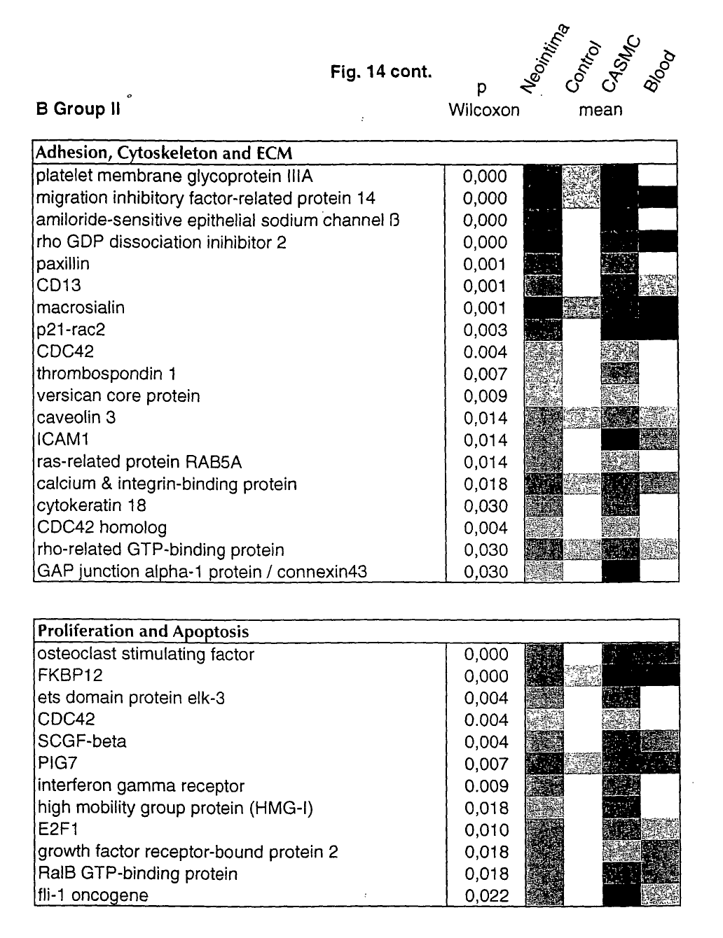

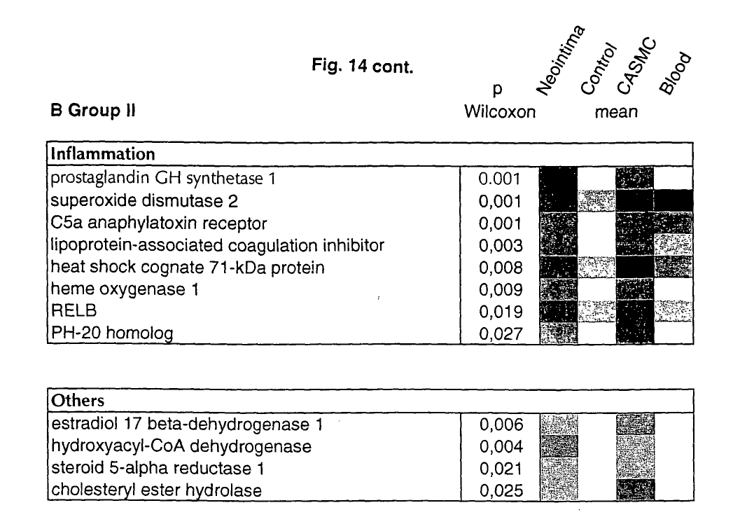

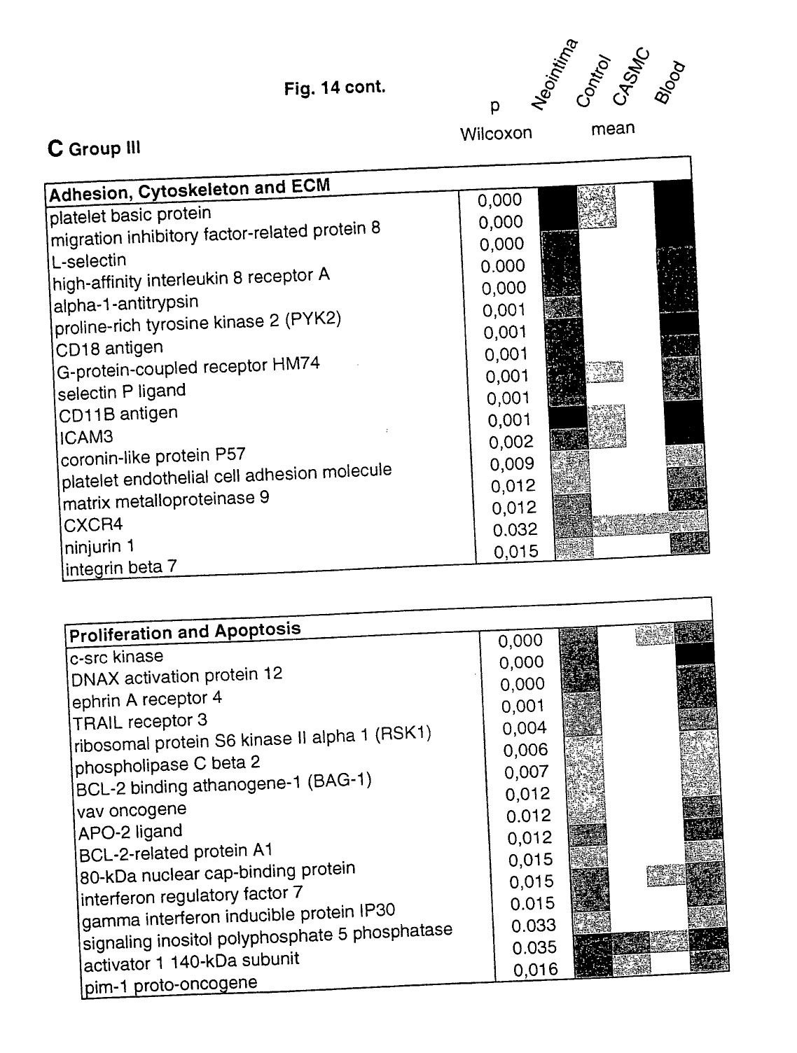

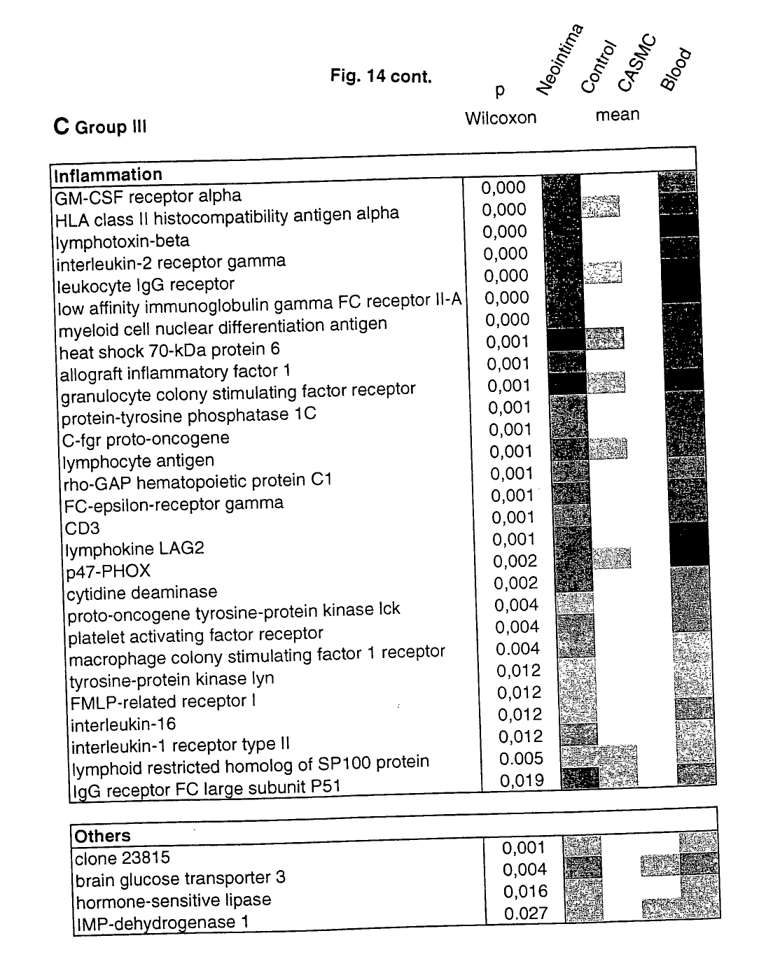

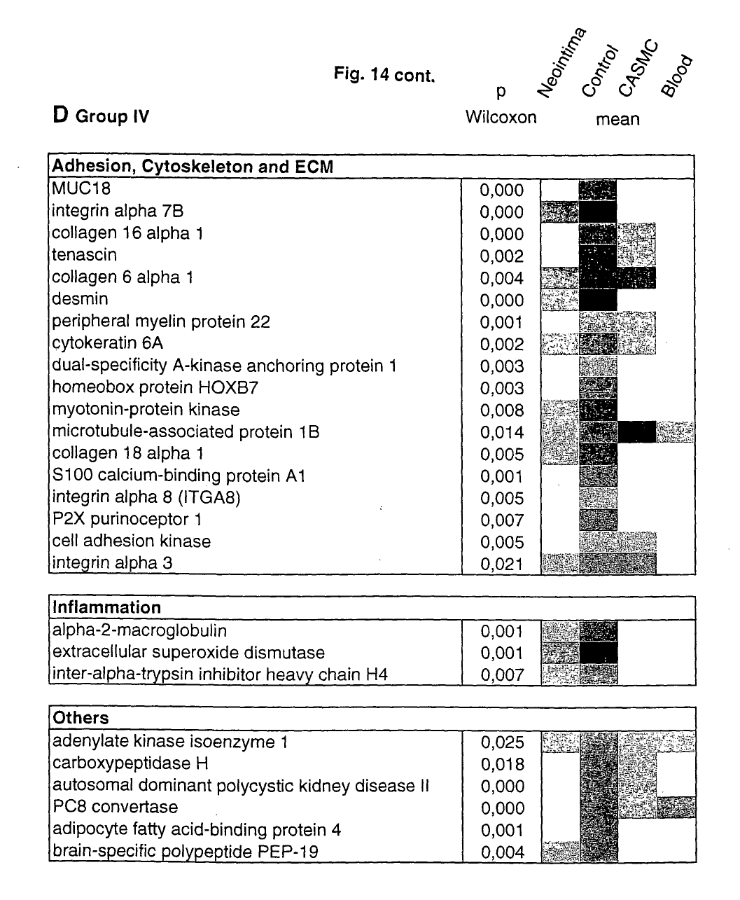

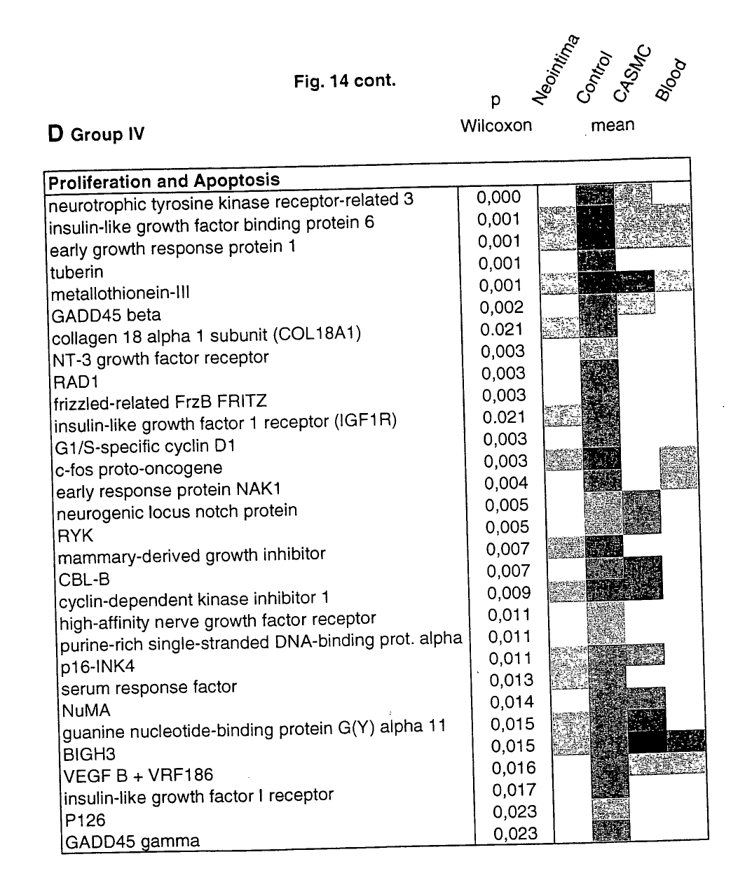

FIG. 14: Cluster image showing the different classes of gene expression profiles of the two hundred twenty four genes whose mRNA levels were different between neointima and control. This subset of genes was clustered into four groups on the basis of their expression in different cell types. The expression pattern of each gene in this set is displayed here as a horizontal strip. Each column represents the average mRNA expression level of the examined group. For each gene, the average of the mRNA level of neointima (n=10), of control (n=11), of proliferating CASMCs (n=2) and of blood samples (n=10) normalized to the mRNA expression level of the housekeeping genes is represented by a color, according to the color scale at the bottom. Group I contained genes only expressed in neointima specimen (FIG. 14A). Group II contained genes expressed simultaneously in neointima and proliferating CASMCs (FIG. 14B). Group III consisted of genes, whose mRNA were expressed in neointima as well as in blood (FIG. 14C). Group IV contained genes, whose mRNA was overexpressed in control specimen (FIG. 14D).

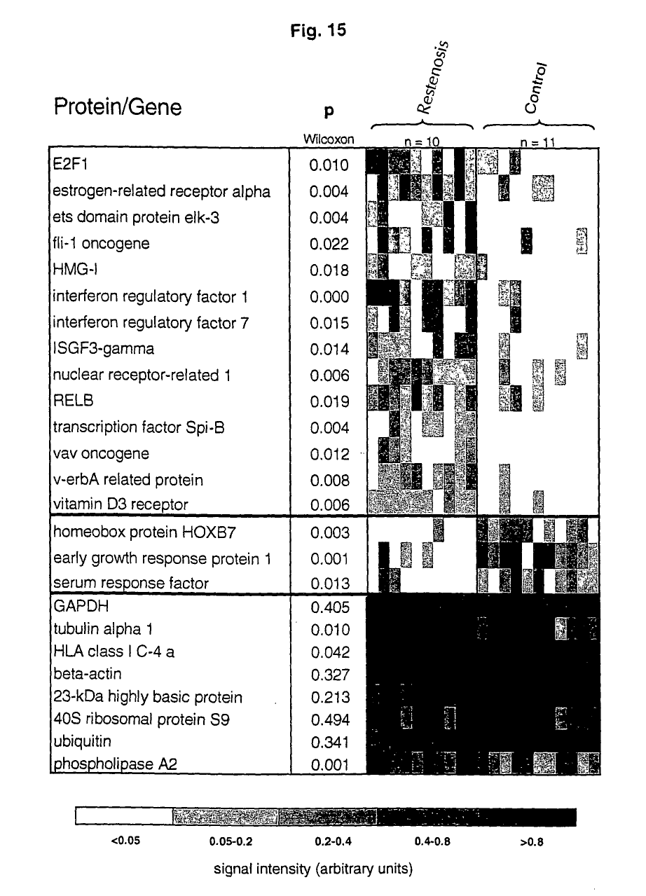

FIG. 15: Expanded view of the transcription factorcluster containing 14 genes that were upregulated in neointima versus control and three transcription factors that were downregulated in neointima. In this case, each column represents a single specimen, and each row represents a single gene

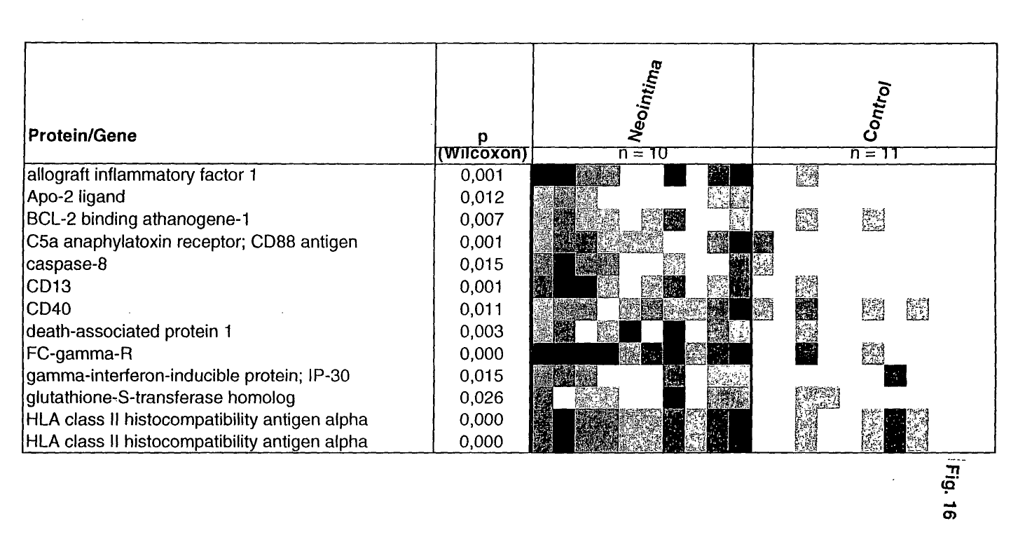

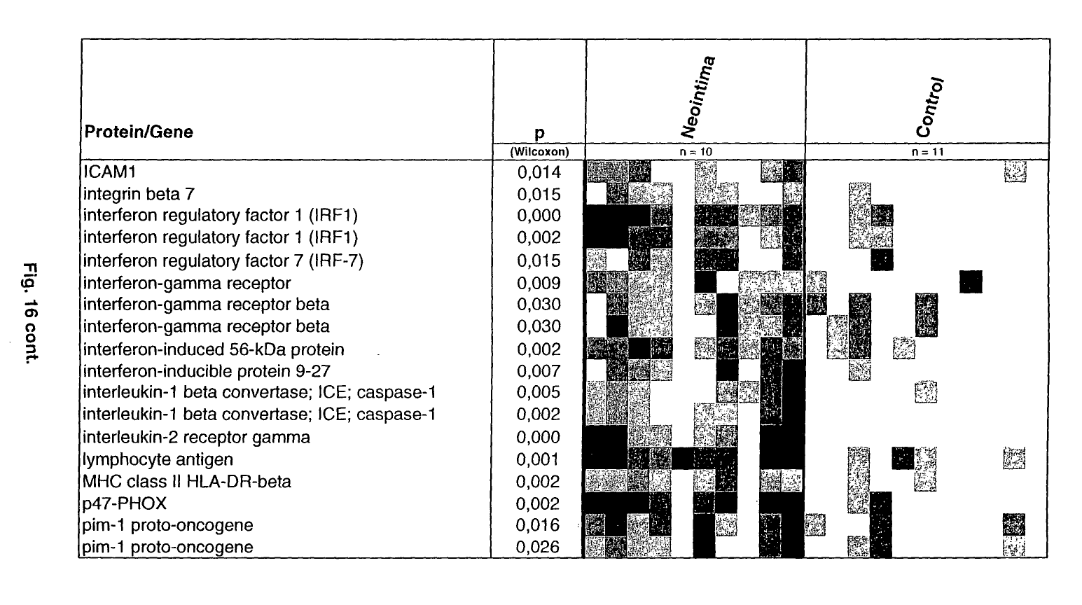

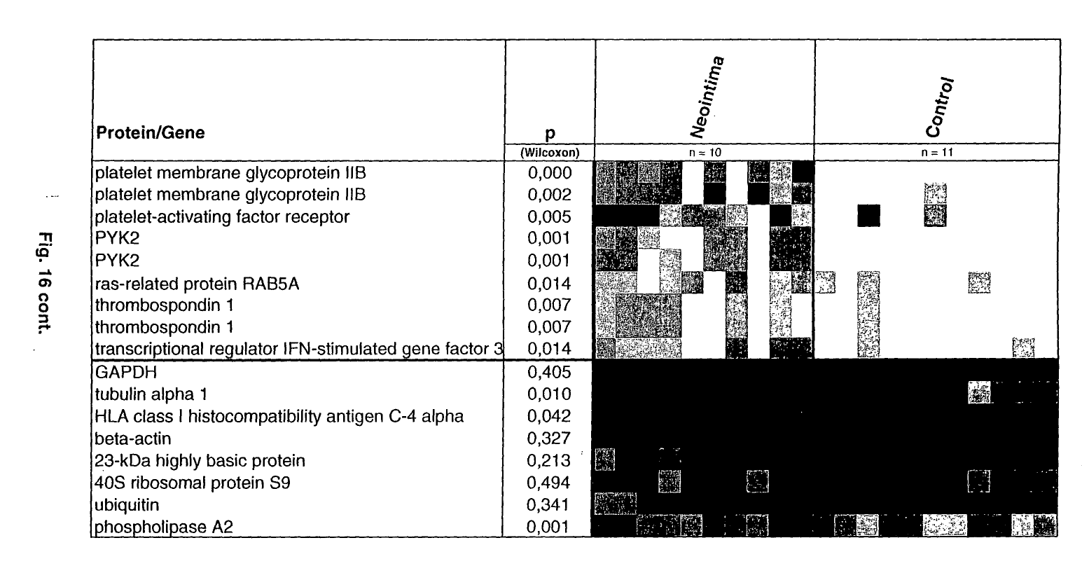

FIG. 16: Expanded view of the IFN-γ-associated cluster containing 32 genes that were upregulated in neointima versus control. In this case, each column represents a single specimen, and each row represents a single gene.

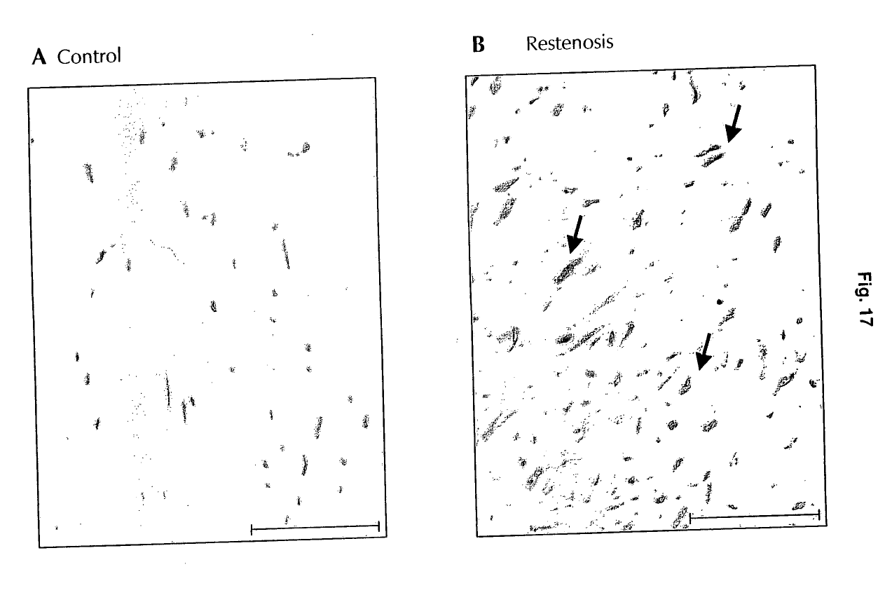

FIG. 17: Immunohistochemical stains of neointima from a carotid restenosis and healthy control media for the IRF-1 protein (left panel: control media; right panel: neointima). The experiment shown is a representative of 6 independent experiments.

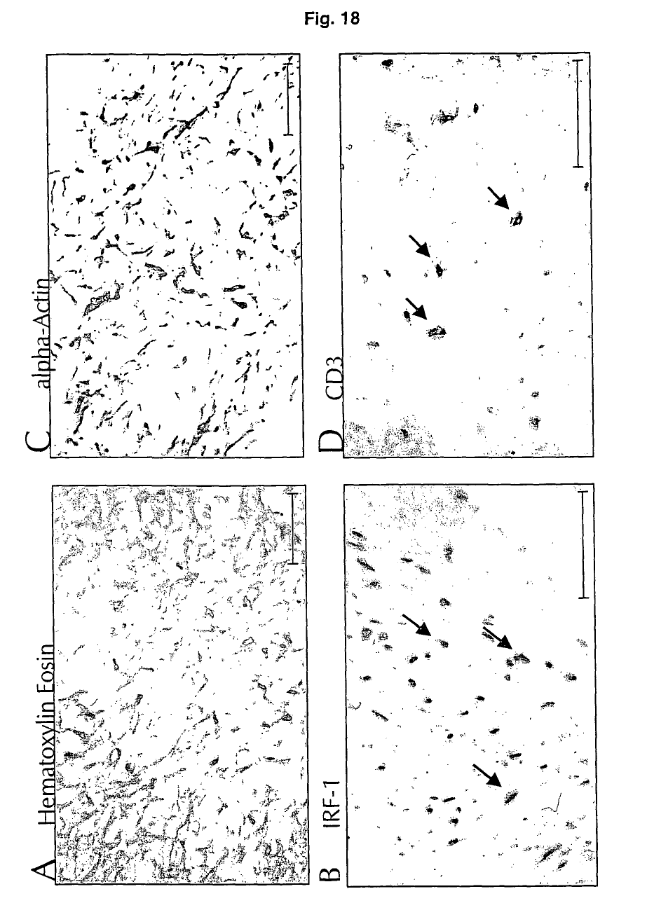

FIG. 18: Immunohistochemical stain of neointima from a coronary in-stent for the IRF-1 protein. Panel A shows a hematoxylin eosin staining of the neointimal specimen from in-stent restenosis, panel B shows a staining for the smooth muscle cell marker α-actin, panel C shows the immunohistochemical stain for the transcription factor IRF-1 in neointima from in-stent restenosis and panel D shows immunohistochemical stain for CD3. The experiment shown here is a representative of three independent experiments.

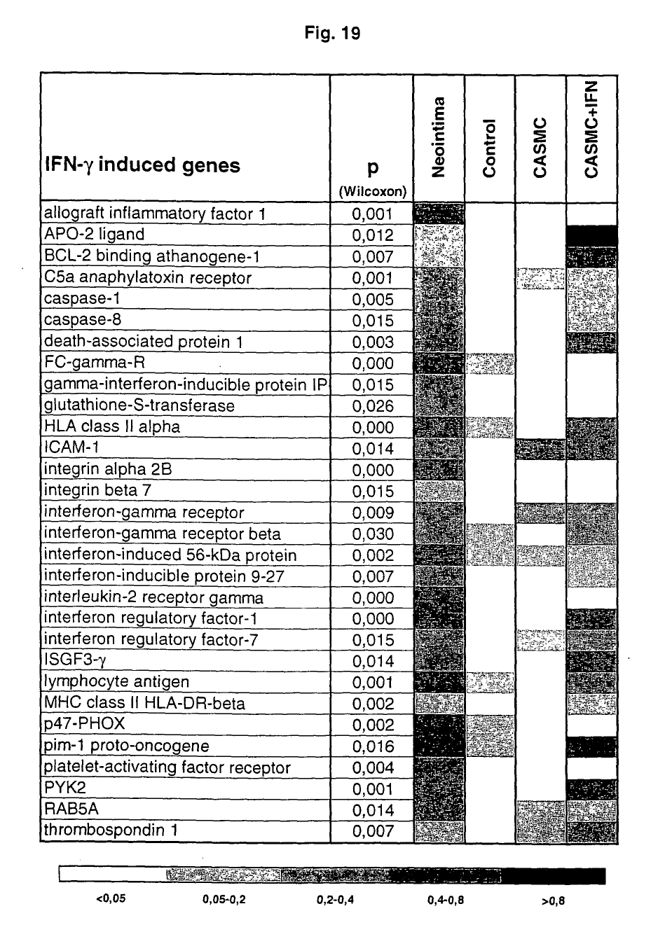

FIG. 19: View of the IFN-γ-associated cluster containing the 32 genes that were upregulated in neointima versus control compared to expression in cultured CASMCs and to cultured CASMCs stimulated for 16 h with 1000 U/mL IFN-γ. In this case, each column represents a single specimen, and each row represents a single gene. One grey value corresponds to a signal intensity as shown at the bottom of the figure.

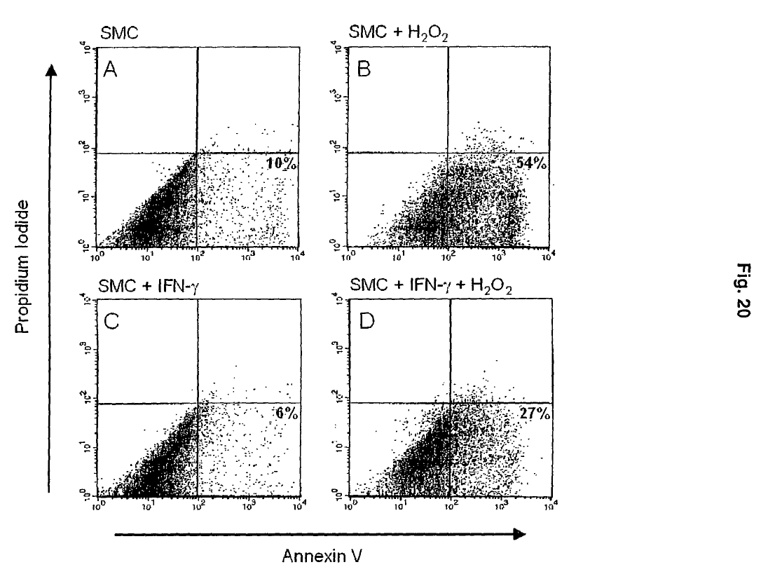

FIG. 20: Effect of IFNγ on survival of cultured SMCs. Flow cytometry analysis of spontaneous (panel A and C) and H₂O₂-induced apoptosis (panel B and D). Cells were double-stained by FITC-labelled Annexin V and PI at 6 h after treatment with 100 μmol/l H₂O₂. A representative analysis of 5 independent experiments is shown.

FIG. 21: The effect of an IFNγ receptor null mutation on the development of neointima in a mouse model of restenosis. (A-D) Representative microphotographs of cross-sectioned mouse carotid arteries from wildtype (wt) and IFN-γR^{-/-} knockout (ko) mice are shown for the untreated artery (control) and the contralateral ligated artery (ligated) at 4 weeks after ligation. The van-Giesson staining procedure was used. The bars represent a lenght of 100 μm. (E) Data from 16 wildtype and 11 IFN-γR^{-/-} mice are shown as mean ±SEM (bars) and analyzed by the t-test for unpaired samples. The scale gives the thickness of media and neointima in μm. Open columns: control animals before and after carotis ligation; filled columns: knockout animals before and after carotis ligation. The shaded area indicates the thickness of neointima.

The invention will now be described by reference to the following biological examples which are merely illustrative and are not to be construed as a limitation of scope of the present invention.

### EXAMPLE I

### Generation and Global Amplification of Single Cell cDNA

The amount of mRNA from single cells is too low for direct use in array-based transcriptome analysis. Total RNA from 50,000 cells (10 μg) was reported to be the detection limit for direct-labelling approaches (Mahadevappa, Nat.Biotechnol., 17, 1134-1136 (1999)). Using a linear amplification step, this number could be reduced to 1000 cells (Luo, Nat. Med., 5, 117-122 (1999)), which is still far beyond applicability for the study of micrometastatic cells. Thus reverse transcription of mRNA and amplification of the cDNA is necessary. Key is the development of an unbiased global amplification procedure. In a simplified manner, this approach consists of four basic steps: (1) isolation of the mRNA on oligo-dT-coated solid support, (2) cDNA synthesis using random primers containing a 5′-oligo-dC (or dG) flanking region, (3) 3′tailing reaction with dGTP (or dCTP) generating a 3′-oligo-dG flanking region, followed by (4) single primer-based amplification using a primer hybridizing to oligo-dG (or -dC) flanking regions of the cDNA molecules. In order to fulfil these four basic steps and to obtain high sensitivity and reliability for cDNA synthesis, 3′-tailing and pCR amplification, tRNA and rRNA had to be removed.

Furthermore, concentrations of random primers were 2000-8000-times higher for cDNA synthesis compared to previously desribed oligo-dT-based approaches (Brady, Methods. Enzymol., 225, 611-623 (1993); Trumper, Blood, 81, 3097-3115 (1993)), who employed 10 nM cDNA synthesis primers. Twenty HT29 colon carcinoma cells (ATCC: HTB-38) were individually isolated and processed. After cell lysis in cDNA synthesis buffer containing the detergent Igepal, groups of five cells were formed and reverse transcribed with four different concentrations of random cDNA synthesis primers. By gene-specific RT-PCR cDNA synthesis was tested for each concentration. FIG. 1a shows that higher concentrations of random primers for cDNA synthesis lead to increased detection rates of specific transcripts (e.g. ki-ras). Surplus primer, being an effective competitor of the subsequent tailing and amplification reaction, was, therefore, preferably removed prior to both steps. Equally, high dNTP concentrations improved cDNA synthesis but interfered with the subsequent tailing reaction and needed to be removed. Standard cacodylate-containing tailing buffer interfered with the following PCR and was replaced with a KH2PO4 buffer of low ionic strength (Nelson, Methods Enzymol., 68, 41-50 (1979). Capturing of mRNA on oligo-dT coated magnetic beads provided for simple handling during mRNA isolation and buffer exchange steps. In the following, the isolation of single cells, mRNA isolation, cDNA synthesis and 3′-tailing is briefly described and exemplified.

Tumor cells were isolated from bone marrow as described (Klein, Proc. Natl. Acad. Sci. USA, 96, 4494-4499 (1999)). Briefly, viable bone marrow samples were stained for 10 min. with 10 μg/ml monoclonal antibody 3B10-C9 in the presence of 5% AB-serum to prevent unspecific binding. 3B10-positive cells were detected with B-phycoerythrin-conjugated goat antibody to mouse IgG (The Jackson Laboratory) and transferred to PCR-tubes on ice. Oligo-dT beads were added, the cells lysed in 10 μl lysis buffer (Dynal), tubes rotated for 30 min. to capture mRNA. 10 μl cDNA wash buffer-1 (Dynal) containing 0.5% Igepal (Sigma) was added and mRNA bound to the beads washed in cDNA wash buffer-2 (50 mM Tris-HCl, pH 8,3, 75 mM KCl, 3 mM MgCl₂, 10 mM DTT, supplemented with 0.5% Tween-20 (Sigma)), transferred to a fresh tube and washed again in cDNA wash buffer-1 to remove any traces of LiDS and genomic DNA. mRNA was reverse transcribed with Superscript II Reverse Transcriptase (Gibco BRL) using the buffers supplied by the manufacturer supplemented with 500 μM dNTP, 0.25% Igepal, 30 μM Cfl5c8 primer (5′-(CCC)₅ GTC TAG ANN (N)₆-3′) and 15 μM CFL5cT (5′-(CCC)₅ GTC TAG ATT (TTT)4 TVN, at 44° C. for 45 min. Samples were rotated during the reaction to avoid sedimentation of the beads. cDNA remained linked to the paramagnetic beads via the mRNA and washed once in the tailing wash buffer (50 mM KH₂PO_{4,}, pH 7.0, 1 mM DTT, 0.25% Igepal). Beads were resuspended in tailing buffer (10 mM KH₂PO₄, pH 7.0, 4 mM MgCl₂, 0.1 mM DTT, 200 μM GTP) and cDNA-mRNA hybrids were denatured at 94° C. for 4 min, chilled on ice, 10 U TdT (MBI-Fermentas) added and incubated at 37° C. for 60 min or 37° C., 60 min and 22° C. over night. After inactivation of the tailing enzyme (70° C., 5 min), PCR-Mix I was added consisting of 4 μl of buffer 1 (Roche, Taq long template), 3% deionized formamide (Sigma) in a volume of 35 μl. The probes were heated at 78° C. in the PCR cycler (Perkin Elmer 2400), PCR Mix II, containing dNTPs at a final concentration of 350 μM, CP2 primer (5′-TCA-GAA-TTC-ATG-CCC-CCC-CCC-CCC-CCC-3′, final concentration 1.2 μM) and 5 Units of the DNA Poly-Mix was added, (Roche, Taq Long Template) in a volume of 5 μl for a hot start procedure. Forty cycles were run at 94° C., 15 sec, 65° C., 30° C., 68° C., 2 min for the first 20 cycles and a 10 sec- elongation of the extension time each cycle for the remaining 20 cycles, and a final extension step at 68° C., 7 min. These PCR amplification conditions differ substantially from Brail, Mut. Res. Genomics, 406, 45-54 (1999). Annealing temperature in Brail is only 42° C. for 2 min in contrast to the 65° C. applied in this example of method of invention.

Tailing efficiency as well as the sensitivity of the subsequent PCR of poly-dA- and poly-dG-tailed sequences was assessed using a defined cDNA fragment with a homopolymer tail of either poly-dA or poly-dT. The poly-(dA) and poly-(dG)-tailed fragments were diluted and then amplified by PCR using equal amounts of poly(dT) and poly(dC) primers, respectively. In these experiments poly-C primers binding to poly-G tails were found to be at least 100-times more sensitivity than poly-T primers on poly-dA tails (FIG. 1b compare lanes 1,2 to 3,4) Various cDNA synthesis primers sharing the same poly-dC flanking region in combination with random hexamers (N6), octamers (N8), oligo-dT (dT)₁₅ alone or in combination were compared. All worked well and reliably. The best results were obtained with a combination of poly-dC-N8 and poly-dC-(dT)₁₅ primers (data not shown).

The most dramatic improvement was obtained when only one primer (FIG. 1c) was used for global PCR instead of two (FIG. 1d). The cDNA synthesis primer consisted of a 3′ random hexamer and flanking region either a poly-dC stretch (CFI5c) or a flanking sequence of all four bases (FI4N6). Two poly-dC binding primers were tested in combination with an additional primer binding to FI4 complementary sequence (FIG. 1c). Use of an additional primer (FL4) to the poly-dC binding primers (CP2, CP3) prevented amplification (FIG. 1c, lanes 1,2 and 4,5). This is likely due to the high primer concentrations required for optimal sensitivity. The use of the CP2 primer alone resulted in amplification of a wide range of cDNA molecules (0.2-3 kB). Even highly diluted cDNA (1:200) was still sufficient for global amplification (FIG. 1d).

### EXAMPLE II

### Transcriptome Analysis of Single Cells: Specificity, Reproducibility, Sensitivity, and Suitability for cDNA Array Analysis

Isolated single cells from cultured cell lines were analyzed by the optimized protocol for cDNA synthesis, tailing and amplification. A total of 100 single cells have so far been successfully tested for β-actin and EF-1α expression by gene-specific PCR (data not shown). cDNAs for housekeeping genes were found in a sufficient copy number per cell to be relatively independent of the region used for specific amplification in the secondary PCR. For less abundant transcripts, it was noted that the size of the chosen coding sequence determined detection rates. Highest sensitivity was obtained with the two primers being separated by less than 200 bp (data not shown).

The PCR amplificates from single cells were tested for suitability of cDNA array analysis. For this purpose, the obtained cDNA was Dig(Digoxigenin)-labeled. Dig-UTP was incorporated by PCR. For expression profiling 0.1-1 μl of the original PCR amplified cDNA fragments were used for reamplification in the presence of digoxigenin-labeled dUTP (Boehringer Mannheim), 50 μM dig-dUTP, 300 μM dTTP, and other dNTPs at a final concentration of 350 μM. Reamplification conditions were essentially as described above, modifications were the use of 2.5 Units of the DNA Poly Mix. Initial denaturation at 94° C. for 2 min. followed by 12 cycles at 94° C., 15 sec, 68° C., 3 min and a final extension time of 7 min. Specific transcripts were detected using 1 μl of a 1/10 dilution of the original PCR to a final volume of 10 μl.

The specificity of the hybridization of digoxigenin-labeled probes is depicted in Table 1, where the expression pattern of genes from single cells of different histogenetic origin are shown. Cells were MCF-7 (ATCC Number HTB-22), A431 (ATCC Number CRL-1555), K-562 (ATCC Number CCL-243), JY (International Histocompatibility Workshop: IHW9287). Only the MCF-7 and A431 cell expressed the cytokeratin genes, markers for their epithelial origin, whereas the erythroleukemia K562 cell and EBV-transformed B cell JY expressed genes of a haematopoetic origin, including CD33, CD37, CD38, and kappa light chain in the B cell. In addition, the testis- and tumor-specific MAGE genes were highly expressed in all cancer cells but not the virally transformed B cell. These data show that single cell PCR amplificates are useful for cDNA array analysis and produce cell type-specific gene expression patterns of single cells.

### Table 1: Expression of Histogenetically Informative Genes by Single Cells Derived From Different Tissues

TABLE 1

| | MCF-7 | A431 | K562 | JY |
|---|---|---|---|---|
| Aktin | + | + | + | + |
| EF-1a | + | + | + | + |
| CK7 | + | + | − | − |
| CK10 | − | + | − | − |
| CK13 | − | + | − | − |
| CK18 | + | + | − | − |
| CK19 | + | + | − | − |
| EGP | + | + | − | − |
| CD33 | − | − | + | + |
| CD37 | − | − | + | + |
| CD38 | − | − | + | − |
| Kappa | − | − | − | + |
| Vimentin | − | + | + | − |
| α-6 Integrin | + | − | − | − |
| β-1 Integrin | + | − | − | − |
| β-2 Integrin | − | − | − | + |
| β-4 Integrin | − | − | + | − |
| β-7 Integrin | − | − | − | + |
| FAK | + | − | − | − |
| Mage1 | + | − | + | − |
| Mage2 | + | + | + | − |
| Mage3 | + | − | + | − |
| Mage6 | + | − | + | − |
| Mage12 | + | + | + | − |

Individual cells grown in culture were isolated, cDNA synthesized, amplified and hybridized to an array of histogenetically informative genes. Cells were from the following cell lines MCF-7 (breast cancer); A431 (epidermoid carcinoma); K562 (chronic myeoloid leukemia); JY (Epstein-Barr virus transformed B cell line).

In order to assess reproducibility, the expression pattern of four MCF-7 cells were compared using a cDNA array Generation 4 with 110 different genes (Table 2). Custom made cDNA arrays were prepared as follows. cDNAs were PCR-amplified with gene-specific primers from human cDNA, PCR amplificates were gel-purified and 15 ng DNA per amplificate was spotted onto nylon membranes (Boehringer) using a BioGrid spotting robotic device (Biorobotics). DNA Macroarrays were termed Generation 4 and Generation 5 (see herein below).

Filter Generation 4: Spotted genes were:

| Protein Name | HUGO Name | Protein Name | HUGO Name |
|---|---|---|---|
| Cytokeratin 7 | KRT7 | slap | SLA |
| Cytokeratin 8 | KRT8 | p21 | CDKN1A |
| Cytokeratin10 | KRT10 | p68 | |
| Cytokeratin13 | KRT13 | p27 | CDKN1B |
| Cytokeratin18 | KRT18 | Eck | EPHA2 |
| Cytokeratin19 | KRT19 | P33 | ING1 |
| Cytokeratin20 | KRT20 | B61 | EFNA1 |
| Emmprin II | BSG | p53 III | TP53 |
| MT1-MMP | MT1-MMP | E-Cad | CDH1 |
| MT2-MMP | MT2-MMP | p53 IV | TP53 |
| MT3-MMP | MT3-MMP | P-Cad | CDH3 |
| MT4-MMP | MT4-MMP | p57 | CDKN1C |
| TIMP1 | TIMP1 | N-Cad | CDH2 |
| TIMP2 | TIMP2 | Cyclin D | CCND1 |
| TIMP4 | TIMP4 | c-myc I | MYC |
| MMP1 | MMP1 | Gas1 | GAS1 |
| uPA | PLAU | c-myc II | MYC |
| uPA-Rezeptor | PLAUR | Ki-67 | MKI67 |
| PAIl | PAIl | RB | RB1 |
| PAI2 | PAI2 | b-Aktin | ACTB |
| CathepsinB | CTSB | HTK | TK1 |
| CathepsinD | CTSD | EF-1a | EEF1A1 |
| CathepsinL | CTSL | RAD 51 | RAD51 |
| Stromelysin1 | MMP3 | A20 | TNFAIP3 |
| Stromelysin3 | MMP11 | Nck | NCK1 |
| Gelatinase A | MMP2 | BCL-2 | BCL2 |
| Gelatinase B | MMP9 | pBS | |
| Matrilysin | MMP7 | GAPDH | GHPDH |
| Cystatin 1 | CSTA | hEST | TERT |
| Cystatin 2 | CSTB | Mage 1 | MAGEA1 |
| Cystatin 3 | CST3 | TSP-1 | THBS1 |
| ADAM 8 | ADAM8 | Mage 3 | MAGEA3 |
| ADAM 9 | ADAM9 | mrp-1 | ABCC1 |
| ADAM 10 | ADAM10 | Mage4 | MAGEA4 |
| ADAM 11 | ADAM11 | mdr-1 | ABCB1 |
| ADAM 15 | ADAM15 | Mage 6 | MAGEA6 |
| ADAM 20 | ADAM20 | DEP-1 | PTPRJ |
| ADAM 21 | ADAM21 | Mage 12 | MAGEA12 |
| TACE | ADAM17 | PTP-μ | PTPRM |
| a4-Integrin | ITGA4 | Mage1F | MAGEA1 |
| a5-Integrin | ITGA5 | Creatin Kinase | CKM |
| a6-Integrin | ITGA6 | Mage2F | MAGEA2 |
| av-Integrin | ITGAV | Mage 4F | MAGEA4 |
| GFP | | Mage3F | MAGEA3 |
| beta-Actin | ACTB | Mage 12F | MAGEA12 |
| b1-Integrin | ITGB1 | CD16 | FCGR3A |
| b2-Integrin | ITGB2 | TGF-a | TGFA |
| b3-Integrin | ITGB3 | CD33 | CD33 |
| b4-Integrin | ITGB4 | TGF-b | TGFB1 |
| b5-Integrin | ITGB5 | CD34 | CD34 |
| b7-Integrin | ITGB7 | VEGF | VEGF |
| p15 | CDKN2B | CD37 | CD37 |
| Fak | PTK2 | IGF-I | IGF1 |
| p16 | CDKN2A | CD38 | CD38 |
| Ramp-1 | | kappa | IGKC |
| CD40 | CD40 | TGF-b R.II | TGFBR1 |
| Ramp-2 | | lambda | IGLC1 |
| CD45 II | PTPRL | IGF-RI | IGFR1 |
| EMM I | BSG | Vimentin | VIM |
| CD83 | CD83 | IGF-RII | IGFR2 |
| GFP | | EGP-1 | M4S1 |
| pBS | | MUC 18 | MCAM |
| erb B2 | ERBB2 | DP-I | DSP |
| TCR | TCRA | PHRIP | PHLDA1 |
| TGF-b Rez.I | TGFBR1 | CEA | CEA |
| | | EF-1a | EEF1A1 |

### Table 2: Commonly and Differentially Expressed Genes of Four Single MCF-7 Cells

TABLE 2

| 4/4 | 3/4 | 2/4 | 1/4 |
|---|---|---|---|
| EF-1a | CK19 | Beta-4-Integrin | CK10 |
| GAPDH | TIMP-1 | Beta-5-Integrin | CK13 |
| b-Actin | Cathepsin B | P53 | ADAM 9 |
| CK7 | Cathepsin D | Creatin Kinase | ADAM 15 |
| CK8 | Cathepsin L | | ADAM17 (TACE) |
| CK18 | ADAM 10 | | p16 |
| CK20 | c-myc | | p21 |
| Alpha 6-Integrin | | | p27 |
| Beta 1-Integrin | | | p33 |
| Fak | | | ki-67 |
| EMMPRIN | | | hTK |
| u-PAR | | | E-cadherin |
| Matrilysin | | | IGF-R I |
| Cyclin D1 | | | IGF-R II |
| Eck | | | TGF-beta |
| EpCAM | | | VEGF |
| Mrp-1 | | | DP-I |
| PHRIP | | | |

Heterogeneity of gene expression of individual cells derived from the same cell clone. Four MCF-7 cells isolated from cell culture were analyzed by single cell analysis of gene expression. Listed are the transcripts that were detected in all four single cells (4/4), three of four (3/4), two of four (2/4), and one of four (1/4). 18/46 (39%) expressed genes were detected in all cells. 61% genes could only found in a portion of the four cells. 63 genes were negative for all cells tested.

46 genes (42%) were expressed in at least one cell and 63 (58%) were negative for all four cells. Eighteen of the 46 (39%) expressed genes were detected in all four cells whereas the remaining 29 (61%) were found to be heterogeneously expressed. To evaluate whether this heterogeneity was due to intercellular variation or is an artifact of the technique, it was tested whether disparity is also observed with the cDNA of a single A431 cell that was split for two separate PCR amplifications. In a first experiment, gene-specific PCRs with the globally amplified PCR products obtained from 50% of single cell cDNA (FIG. 2) were performed. For comparison, cDNA isolated from a pool of 500.000 A431 cells were diluted to such an extent that the intensity of the β-actin band was similar to that obtained with 50% of the single cell cDNA. After 32 cycles and with a cDNA amount corresponding to about 10.000 cells, the β-actin signal of the pool control and 50% of the single cell cDNA reached the plateau phase of amplification. As shown in FIG. 2, the variation between two cDNA halves of the same cell was very low. In two independent experiments, each half (a+b) from six A431 cells yielded β-actin bands of similar intensity.

In order to test the reliability of the global amplification of the cDNA, a second gene sequence-specific PCR amplification was performed. As the efficiency of gene-specific PCR amplification is known to be primer sequence-dependent, the amplification of MAGE transcripts was tested, which are very demanding with respect to primer design (Kufer, WO98/46788 (1998); Serrano, Int. J. Cancer 83, 664-669 (1999)). The level of MAGE expression determined by sequence specific PCR was consistently lower than that of beta-actin. The relative abundance of MAGE transcripts in split single cell samples after global PCR amplification of the cDNA (FIG. 2, lanes 2-4 and 6-8) was comparable to that of the control sample from unamplified cDNA from pooled cells (FIG. 2, +). In 4 out of 6 cases, the results were identical for both halves of the cDNA. The lack of any MAGE transcript in cell half 7a and 8b most likely indicates an unequal distribution of the cDNAs between the two halves.

The observed sequence-independent amplification is characteristic of the poly-dC primer, which contains fifteen cytosine residues and therefore introduces primer binding sites with equally high CG-content. The experimental conditions suited for such a primer, i.e. high annealing temperature (65° C.) in the presence of 3% denaturing formamide, lead, to a remarkable reproducibility and did not introduce major quantitative changes to the single cell transcriptome.

Amplificates from split single cell cDNAs and, as control, cDNA from 5,000 pooled cells were labeled and hybridized to a cDNA array representing 193 different genes. Most transcripts could be detected with both halves of the single cell amplificates (Tab. 3).

### Table 3: Gene Expression Patterns of Single Cells Split in Two Pools of cDNA Prior to Global PCR Compared to Pooled cDNA of 5000 Cells

TABLE 3

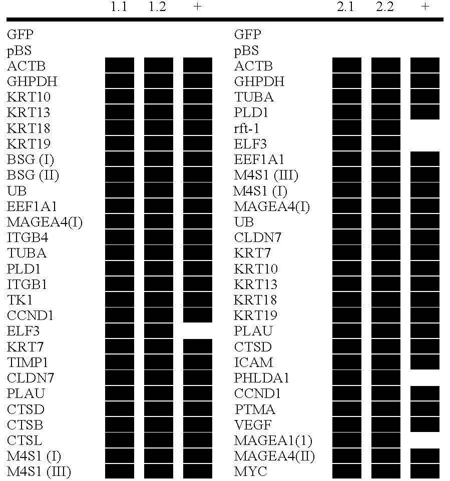

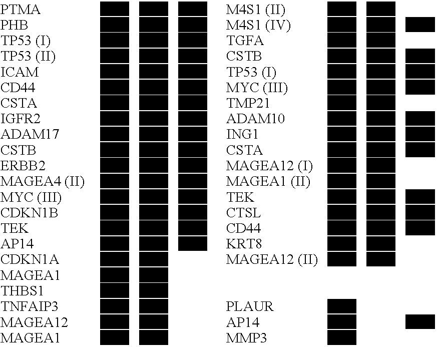

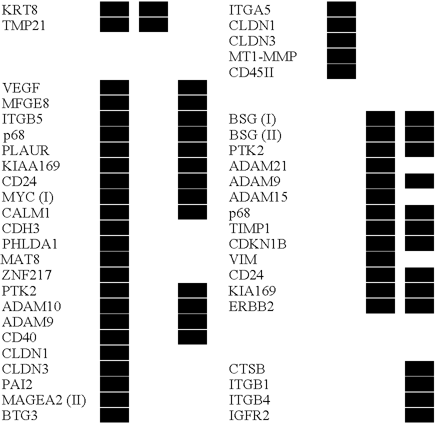

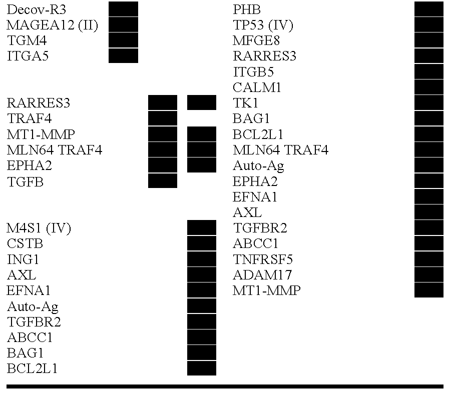

The cDNAs of two single cells were split prior to PCR amplification and compared to a cDNA pool derived from 5000 cells. All cDNAs were amplified by global PCR and analyzed by hybridization to a cDNA array. The gene expression profiles of the corresponding halves (1.1 and 1.2; 2.1 and 2.2) are juxtaposed to the cell pool (+). The genes are listed according signal strength (the darker, the stronger) and detection in both halves of the same cell. The filter used was Generation 5, genes and protein names are listed below (for preparation of said Generation 5 filter, see herein above (Generation 4 filter)).

Generation 5 Filter:

| Protein | HUGO |
|---|---|
| A20 | TNFAIP3 |
| a4-Int | ITGA4 |
| a5-Int | ITGA5 |
| a6-Int | ITGA6 |
| ADAM10 | ADAM10 |
| ADAM15 | ADAM15 |
| ADAM21 | ADAM21 |
| ADAM9 | ADAM9 |
| Auto-Ag | SHGC-74292 |
| av-Int | ITGAV |
| Axl | AXL |
| b1-Int | ITGB1 |
| b2-Int | ITGB2 |
| b3-Int | ITGB3 |
| b4-Int | ITGB4 |
| b5-Int | ITGB5 |
| B61 | EFNA1 |
| b7-Int | ITG7 |
| BA46 | MFGE8 |
| BAG1 | BAG1 |
| b-Aktin | ACTB |
| b-Casein | CSN2 |
| Bcl-2 | BCL2 |
| Bcl-xl | BCL2L1 |
| b-micro | MSMB |
| BTG-3/ANA | BTG3 |
| Calmodulin | CALM1 |
| Cathepsin B | CTSB |
| Cathepsin D | CTSD |
| Cathepsin L | CTSL |
| CD16 | FCGR3A |
| CD24 | CD24 |
| CD33 | CD33 |
| CD34 | CD34 |
| CD37 | CD37 |
| CD38 | CD38 |
| CD40 | TNFRSF5 |
| CD44 | CD44 |
| CD45 | PTPRC |
| CD83 | CD83 |
| CEA | CEA |
| CK10 | KRT10 |
| CK13 | KRT13 |
| CK18 | KRT18 |
| CK19 | KRT19 |
| CK7 | KRT7 |
| CK8 | KRT8 |
| Claud1 | CLDN1 |
| Claud3 | CLDN3 |
| Claud7 | CLDN7 |
| c-myc | MYCBP |
| Cyclin D1 | CCND1 |
| Cystatin A | CSTA |
| Cystatin B | CSTB |
| Decoy-R2 | TNFRSF10D |
| Decoy-R3 | TNFRSF6B |
| DEP-1 | PTPRJ |
| DP-1 | DSP |
| E2F6 | E2F6 |
| E-Cad | CDH1 |
| Eck | EPHA2 |
| EF1a | EEF1A1 |
| EGP1 | M4S1 |
| Emmprin | BSG |
| EPC-1 | PEDF |
| erbB2 | ERBB2 |
| Ese1b/ELF3 | ELF3 |
| Fak | PTK2 |
| FGFR1 | FGFR1 |
| FGFRII | FGFR2 |
| Gadd45 | GADD45A |
| GAPDH | GHPDH |
| Gas1 | GAS1 |
| Gas6 | GAS6 |
| GFP | |
| hEST | TERT |
| Hevin | HEVIN |
| HTK | TK1 |
| ICAM | ICAM |
| IGF RI | IGFR1 |
| IGF RII | IGFR2 |
| Kappa | IGKC |
| Ki67 | MKI67 |
| KIA169 | |
| Lambda | IGLC1 |
| lot1/hZAC | Hs. 75825 |
| Mage1 | MAGEA1 |
| mage12 | MAGEA12 |
| Mage2f | MAGEA2 |
| Mage4 | MAGEA4 |
| MAT8 | PLML |
| Mdr-1 | ABCB1 |
| MLN62 | TRAF4 |
| MLN64 TRAF4 | |
| mrp-1 | ABCC1 |
| MT1-MMP | MT1-MMP |
| Muc 18 | MCAM |
| N-Cad | CDH2 |
| Nck | NCK1 |
| p15 | CDKN2B |
| p16 | CDKN2A |
| p21 | CDKN1A |
| p27 | CDKN1B |
| p33 | ING1 |
| p53III | TP53 (III) |
| p53IV | TP53 (IV) |
| p57 | CDKN1C |
| p68 | |
| PAI-2 | PAI2 |
| pBS | |
| P-Cad | CDH3 |
| Phospholipase | PLD1 |
| Phrip | PHLDA1 |
| PIP | PIP |
| Prohibitin | PHB |
| Prost.Spec.Homeo. | Hs. 73189 |
| Prost.Spec.Transglu | TGM4 |
| Prost.Spec.Uro. | UPK3 |
| Prothym alpha | PTMA |
| PSA | KLK3 |
| PTHrP | |
| PTP-μ | PTPRM |
| RB | RB1 |
| rfx-1 | RFX1 |
| Slap | SLA |
| Stromelysin 1 | MMP3 |
| Survivin | API4 |
| TACE | ADAM17 |
| TCR | TCRA |
| TGF-alpha | TGFA |
| TGF-beta | TGFB1 |
| TGFB-RI | TGFBR1 |
| TGFB-RII | TGFBR2 |
| TIE-2/Tek | TEK |
| TIG3 | RARRES3 |
| Timp1 | TIMP1 |
| TMP21 | TMP21 |
| TSP-1 | THBS1 |
| Tubulin-a | TUBA |
| Ubiquitin | UB |
| uPA | PLAU |
| uPA-R | PLAUR |
| VEGF | VEGF |
| Vimentin | VIM |
| VLDLR | VLDLR |
| ZNF217 | ZNF217 |
| | Hs. 46452 |

A total of 148 signals were obtained for the four cDNA halves. Of these, 95 (64%) were found in the corresponding halves, whereas 53 (36%) were found in only one half. Out of the 53 single positive signals 46 (87%) represented very low-abundant transcripts, with 26 (49%) not detectable and 20 (37%) only weakly expressed in the control of pooled cells. Seven genes (AXL, BAG1, BCL2L1, SHGC-74292, B61, TGFBR2 and ABCC1) were exclusively detected in the pooled sample, though with a rather weak signal. In contrast, 33 genes were only found in the half-cell experiments but not in the control. The signal intensity of the both halves was quite similar, with 55% and 76% of the signals having the same strength in the corresponding halves. Signals that were not identical in two corresponding halves may arise from of a non-random distribution of cDNA fragments prior to PCR. Particularly transcripts present in low (<10) copy number may be subject to such a distribution effect which, however, may not be obtained if samples are not split.

### EXAMPLE III

### Combined Transcriptome and Genome Analysis from Single Cells

A method of CGH (comparative genomic hybridization) analysis of single cells (SCOMP) was recently described (Klein, Proc. Natl. Acad. Sci. USA, 96, 4494-4499 (1999)). Using this method, a tumor cell can unambiguously be identified by its chromosomal aberrations. It was therefore attempted to isolate both genomic DNA and mRNA from the same cell. Isolated single cells were lysed in 10 μl lysis buffer (Dynal) and tubes rotated for 30 min. to capture mRNA. 10 μl cDNA wash buffer-1 (50 mM Tris-HCl, pH 8,3, 75 mM KCl, 3 mM MgCl₂, 10 mM DTT, supplemented with 0.5% containing 0.5% Igepal (Sigma)) was added and mRNA bound to the beads washed in cDNA wash buffer-2 (50 mM Tris-HCl, pH 8,3, 75 mM KCl, 3 mM MgCl₂, 10 mM DTT, supplemented with 0.5% Tween-20 (Sigma)), transferred to a fresh tube and washed again in cDNA wash buffer-1 to remove any traces of LiDS and genomic DNA. mRNA was reverse transcribed with Superscript II Reverse Transcriptase (Gibco BRL) using the buffers supplied by the manufacturer supplemented with 500 μM dNTP, 0.25% Igepal, 30 μM Cfl5c8 primer (5′-(CCC)₅ GTC TAG ANN (N)₈-3′) and 15 μM CFL5cT (5′-(CCC)₅ GTC TAG ATT (TTT)₄ TVN, at 44° C. for 45 min. Samples were rotated during the. reaction to avoid sedimentation of the beads. Primers used and mentioned in FIGS. 1c and d were Cfl5cN6 (5′-(CCC)₅ GTC TAG ANN (N)₆-3′) and FL4N6 5′-TTT CTC CTT AAT GTC ACA GAT CTC GAG GAT TTC (N)₆-3′). cDNA remained linked to the paramagnetic beads via the mRNA and washed once in the tailing wash buffer (50 mM KH₂PO_{4,}, pH 7.0, 1 mM DTT, 0.25% Igepal). Beads were resuspended in tailing buffer (10 mM KH₂PO₄, pH 7.0, 4 mM MgCl₂, 0.1 mM DTT, 200 μM GTP) and cDNA-mRNA hybrids were denatured at 94° C. for 4 min, chilled on ice, 10 U TdT (MBI-Fermentas) added and incubated at 37° C. for 60 min or 37° C., 60 min and 22° C. over night. After inactivation of the tailing enzyme (70° C., 5 min), PCR-Mix I was added consisting of 4 μl of buffer 1 (Roche, Taq long template), 3% deionized formamide (Sigma) in a volume of 35 μl. The probes were heated at 78° C. in the PCR cycler (Perkin Elmer 2400), PCR Mix II, containing dNTPs at a final concentration of 350 μM, CP2 primer (5′-TCA-GAA-TTC-ATG-CCC-CCC-CCC-CCC-CCC-3′, final concentration 1.2 μM) and 5 Units of the DNA Poly-Mix was added, (Roche, Taq Long Template) in a volume of 5 μl for a hot start procedure. Forty cycles were run at 94° C., 15 sec, 65° C., 30° C., 68° C., 2 min for the first 20 cycles and a 10 sec- elongation of the extension time each cycle for the remaining 20 cycles, and a final extension step at 68° C., 7 min. PCR primers used in FIG. 1c were CP3 (5′-GCT GAA GTG GCG AAT TCC GAT GCC (C)₁₂-3′) and FL4 (5′-CTC CTT AAT GTC ACA GAT CTC GAG GAT TTC-3′).

The supernatants from the cell lysis and all washing steps (cDNA wash buffer 1 and 2) of the mRNA isolation were collected (total volume 60 μl). After transfer to a silanised tube the genomic DNA was ethanol precipitated overnight at −20° C. in the presence of 20 μg glycogen (Roche). All subsequent steps were performed as published (Klein, (1999), loc. cit.).

A major concern was incomplete precipitation of genomic DNA eventually leading to losses of DNA as seen with chromosome deletions in cancerous cells. However, experiments with cells of a defined karyotype clearly showed that either the cellular DNA was totally lost (30% of cases) or completely precipitated (70%) (data not shown). The complete recovery of genomic DNA may be due to the fact that interphase chromosomes are extensively interwoven so that either all or none is precipitated. The loss of all DNA is probably introduced by the change of reaction tubes during the separation of genomic DNA and mRNA. The karyotypes of two normal and two MCF-7 breast cancer cells whose DNA had been precipitated are shown in FIG. 3. The profiles of the two normal cells showed no significant deviation from the midline while the multiple genomic aberrations of the two MCF-F7 cells were almost identical. Hence, malignant EpCAM-positive cells can be unambiguously distinguished by their genomic phenotype from normal EpCAM-positive cells in the bone marrow. This is of particular importance since EpCAM-expression is insufficient proof for the (malignant) identity of tumor cell(s) in bone marrow samples. It has to be noted that healthy donors also showed 0.5-5‰″3 3B1O-C9-positive cells (3B10-C9, Prof. Judy Johnson, Institute for Immunology, Munich) is a high affinity mAb against EpCAM) when determined by immunofluorescence.

### EXAMPLE IV

### Activity-related Gene Expression in Three Micrometastatic Cells

Single tumor cells were isolated from three patients with different tumors and disease stages. The first patient (C) had a 10-year history of cervical carcinoma and presented with a suspicious finding on chest x-ray. In the second patient (L), an adenocarcinoma of the lung had recently been diagnosed which was post-operatively staged as pT2, N3, M0. The bone marrow sample was obtained during the anesthesia prior to the operation. The third sample was aspirated from the pelvic crest of a 31-year old breast cancer patient (B) whose disease was in the stage pT1a, pN1a (1/18), M0. Because of a local relapse, the histological G3 grading, and finding of one cytokeratin-positive cell in the bone marrow, this patient received high-dose chemotherapy (HD). The bone marrow sample was taken one month after completion of HD. SCOMP was performed with all three cells and showed multiple chromosomal aberrations verifying the cancerous origin of cells (Tab. 4).

### Table 4: Genomic Aberrations of 3B10-C9-positive Cells Isolated From Bone Marrow of a Three Patients With Cervical Carcinoma (C), Lung Cancer (L) and Breast Cancer (B)

TABLE 4

| Cell | 1p | 1q | 2p | 2q | 3p | 3q | 4p | 4q | 5p | 5q | 6p | 6q | 7p | 7q | 8p | 8q | 9p | 9q | 10p | 10q | 11p | 11q | 12p | 12q | 13q |
|---|---|---|---|---|---|---|---|---|---|---|---|---|---|---|---|---|---|---|---|---|---|---|---|---|---|
| C | | G | | | | G | | | | G/L | | | | | | | | L | G | | | | | L | |
| L | L | G | | | L | | | L | G | L | G | G | G | G | L | G | L | | L | | L | G | | | L |
| B | | | | G | | | | G | | | | | | | | | L | L | | | L | L | G | G | |

| Cell | 14q | 15q | 16p | 16q | 17p | 17q | 18p | 18q | 19p | 19q | 20p | 20q | 21p | 21q | 22p | 22q | Xp | Xq | Y |
|---|---|---|---|---|---|---|---|---|---|---|---|---|---|---|---|---|---|---|---|
| C | L | | | L | | | G | | | | L | L | | | | L | | | |
| L | | L | L | G | L | L | | | | | | | | G | | | G | G | |
| B | G | | | | | | | | | | | | | | | | G | G | |

Summary of the CGH-data obtained from the three micrometastatic cells. Losses (L) and gains (G) on the small (p) and long (q) arm of each chromosome are given for each cell.

The cell from patient B, who had the least advanced disease, showed the lowest extent of chromosomal changes (FIG. 4).

mRNA was isolated from all three cells and samples generated for SCAGE as described above. As control, the procedure was performed without the addition of a cell. cDNA amplificates were hybridized to Clontech Cancer 1.2 filters and to newly generated arrays (Axxima A6, Martinsreid) comprising a total of 1,300 genes.

Non-radioactiv hybridization to nylon filters was carried out as follows:

15 ng of the different PCR-amplified and subcloned cDNA fragments were spotted on positively charged nylon filters by Axxima AG, Martinsried. Filters were. pre-hybridized overnight in the presence of 50 μg/ml E. coli and 50 μg/ml pBS DNA in 6 ml Dig-easy Hyb buffer (Roche Biochemicals). 9 μg of labeled PCR products from single cells were mixed with 100 μg herring sperm, 300 μg E. coli genomic DNA and 300 μg, denatured for 5 min at 94° C., added to 6 ml Dig-easy hybridization buffer and hybridized for 36 hours. Stringency washes were performed according to the Roche digoxigenin hybridization protocol adding two final stringency washes in 0.1×SSC +0.1% SDS for 15 min at 68° C. Detection of filter bound probes was performed according to the Digoxigenin detection system protocol supplied with the kit (Roche).

Only three genes had to be excluded from analysis because a signal was obtained in at least one of the negative controls. These genes were the VHL-binding protein, caspase 10, TGF-β and hemoglobin α. The number of positive signals ranged from 5.3% (70/1313), 7.0% (92/1313) to 11.8% (155/1313) for cells from patients B, C, and L respectively. These numbers were considerably lower than those from single in vitrogrown carcinoma cells where signals were obtained with 10-20% of genes (data not shown). All three tumor cells expressed genes known to play a role in regulation of proliferation, replication or growth arrest (FIG. 5; Tab. 5).

### Table 5: Upregulated Genes Implied in Cell Cycle Status in Cells C, L and B

TABLE 5

| Role in cell cycle | C | B | L |
|---|---|---|---|
| Positive | RFC3 | RFC3 | RFC3 |
| regulators | LIG1 | LIG1 | |
| | STK12 | STK12 | |
| | P2G4 | P2G4 | |
| | RFC2 | RFC2 | |
| | ADPRT | ADPRT | |
| | S100A4 | S100A4 | |
| | CCNA (cyclin A) | CDC25 | |
| | MKI67 (Ki-67) | VRK2 | |
| | CENPF | DYRK4 | |
| | D123 | PRIM1 | |
| | PIN1 | PRKDC | |
| | | (DNA-PK) | |
| | EB1 | CHD3 | |
| | CDC27HS | | |
| | CALM1 | | |
| | UBL1 | | |
| | TOP2A | | |
| | HMGIY | | |
| | HDAC3 | | |
| | RBBP4 | | |
| Negative | | CDKN1A | CDKN1A (P21) |
| | | (P21) | |
| regulators | | ING1 | ING1 |
| | | DDIT1 | CDKN2A (P16) |
| | | (GADD45) | |

Cells C and B expressed several positive regulators of the cell cycle, while only B and L expressed cell cycle inhibitors.

Cell C expressed the highest number of genes important for cell cycle progression, including cyclin A (CCNA), EB1, RC2, P2G4, PIN1, RBBP4 and CENPF. As most of these genes are tightly transcriptionally regulated and their mRNAs are rapidly degraded as cell division progresses, their expression not only indicates that cell C was engaged in cycling but can be faithfully captured in this activity by SCAGE.

Cell B expressed a number of genes important for replication as well as cell cycle inhibition. The pattern of transcripts suggests that the cell was in a state of DNA repair. The coexpression of GADD45 (DDIT1) and p21 (CDKN1A) are indicative for growth arrest (Smith, Science, 266, 1376-1380 (1994)). Likewise, the expression of positive cell cycle regulators such as DNA-PK, RFC2, LIG1, ADPRT and PRIM1 has been implicated in DNA repair (Lindahl, Science, 286, 1897-1905 (1999); Barnes, Cell, 69, 495-503 (1992), Mossi, Eur. J. Biochem., 254, 209-216 (1998); Lee, Mol. Cell Biol. 17, 1425-1433 (1997)). As this cell survived an alkalyting, genotoxic high dose chemotherapy its expression profile may be interpreted as if re-entry into cell cycle was obviated. This interpretation is supported by the expression of pro-apoptotic genes such as caspase-6 and BAD that were only found with this cell. Execution of apoptosis in this cell may however be counteracted by expression of survivin (API4) (FIG. 5; Tab. 5).

The transcriptome obtained from cell L showed traits compatible with its engagement in dissemination and EMT. While gene expression of cell L did not resemble that of a cycling or DNA-repairing cell (see above) its 84 differentially expressed genes are mostly involved in cytoskeletal reorganization, cell adhesion and extracellular proteolytic activity (Tab. 6; FIG. 6).

### Table 6: Upregulated Genes in Cell L Indicative for an Invasive Phenotype

TABLE 6

| cytoskeletal organization | Adhesion | proteolytic activity |
|---|---|---|
| Cytokeratin 2 | Integrin alpha 3 | Cathepsin B |
| Cytokeratin 6 | Integrin alpha v | Cathepsin D |
| Cytokeratin 7 | Integrin beta 2 | Cathepsin L |
| Cytokeratin 8 | Integrin beta 3 | MMP7 |
| Cytokeratin 10 | Integrin beta 7 | MT1-MMP |
| Cytokeratin 13, 15, 17 | | MT2-MMP |
| Cytokeratin 18 | Cytohesin 1 | uPA |
| Cytokeratin 19 | Focal adhesion kinase | uPA-R |
| Vimentin | Desmoglein 2 | ADAM 8 |
| Beta-actin | E-cadherin | ADAM 15 |
| | CD9 | ADAM 17 |
| RhoA | | Bikunin |
| RhoB | | |
| Rho-GDI2 | | Cystatin 2 |
| A-raf | | EMMPRIN |
| RAP-1A | | |
| Cdc42 | | |
| Rac1 | | |
| P160 ROCK | | |
| Ste20-like kinase | | |
| Beta-catenin | | |

The present study analyzed for the first time cellular activities of individual tumor cells derived from the bone marrow of cancer patients. Cell C was derived from a cervical carcinoma patient who presented with lung metastasis after a ten-year latent period. This cell was found in proliferation. Cell B was from bone marrow of a breast cancer patient with a rather small primary cancer who had received high dose chemotherapy because of the apparent aggressiveness of her tumor. This cell showed relatively few and discrete genomic changes, a finding that is of particular interest with regard to the genomic changes required for dissemination. Moreover, this cell must have survived four cycles of a regular chemotherapy consisting of Epirubicin and Taxol in addition to a high-dose chemotherapy regimen involving alkylating agents. The obtained expression profile is diagnostic for growth arrest and ongoing DNA repair.

Most informative with respect to the process of dissemination was the transcriptome of cell L. Detected in a bronchial cancer patient without clinically manifest metastasis, this cell expressed many genes encoding proteins involved in active migration and invasion. Most of the activation cascade of the uPA system was found expressed, consisting of the cathepsin B, D, L, the uPA receptor and uPA itself. Likewise, genes involved in organizing filopodia, lamellipodia and stress fibers, the Rho family members RhoA and B, Rac1, Cdc42 and p160 rock, and genes encoding several adhesion molecules were upregulated in this cell. Its cytoskeleton seemed to undergo remodeling as shown by expression of many cytokeratins and vimentin, a marker for EMT.

It is noteworthy that the number of transcripts in single cells isolated from cultured cell lines was considerably lower than that from patient-derived tumor cells. This difference may speak for a tighter in vivo control of transcription that may become more relaxed when cells are grown in cell culture, e.g., by increased DNA demethylation. Expression analysis of ex-vivo specimen might therefore be much more informative than studies on cell lines. The minimal number of cells that has been used for cDNA array analysis so far was in the range of 1,000 cells (Luo (1999), loc. cit.). The sensitivity of the array hybridization might be further increased by longer immobilized cDNA fragments (fragment length on Clontech arrays is about 200 bp), and the amount of information obtained by using glass chips with higher density and complexity. Although the present study analyzed only 1,300 genes, one has to consider that expression of only nine proteins has thus far been reported for micrometastatic cells. These proteins are ErbB2, transferrin receptor, MHC class I, EpCAM, ICAM-1, plakoglobin, Ki-67, p120 and uPA-receptor/CD87 (Pantel, J. Natl. Canc. Inst. 91, 1113-1124 (1999)).

The here described method has potential for the study of gene expression by rare cells in many other fields (as shown hereinbelow; for example, in the investigation of human restenotic tissue). For instance, the investigation of spatially and temporally regulated gene expression in embryogenesis and the analysis of stem cells and differentiated cells in adult tissues could be performed. Single cell analysis would greatly advance the understanding of atypical proliferation, metaplasia, pre-neoplastic lesions and carcinomata in situ.

A synopsis of genomic aberrations and the expression profiles of the same cell may reveal the contingencies of different genotypes and phenotypes within a tumor cell population.

High-dose chemotherapy, surgery, and anti-angiogenic therapy approaches can target rapidly dividing cells and large tumor masses but are ineffective in the elimination of remnant cells leading to minimal residual disease. Adjuvant therapies, like antibody-based approaches Riethmuller, J. Clin. Oncol., 16, 1788-1794 (1998), are still based on protein targets identified on the primary tumor. The here shown approach provides now an opportunity to discover targets for minimal residual disease by analyzing the micrometastatic cells directly.

### EXAMPLE V

### Aberrant Gene Expression in Human Restenotic Tissue

The above described method was furthermore employed to detect differentially expressed genes in human restenotic tissue.

A high rate of restenosis is significantly limiting the success of percutaneous transluminal coronary angioplasty with subsequent stent implantation as a frequent treatment of coronary atherosclerotic disease. Although several cellular and molecular mechanisms have been identified in the development of in-stent restenosis, specific targets for an effective therapeutic prevention of restenosis are still scarce. In this study differentially expressed genes in microscopic atherectomy specimen from human in-stent restenosis were identified. Immunohistochemistry showed that the restenotic material consisted mainly of smooth muscle cells (SMC) with rare infiltrates of mononuclear cells. cDNA samples prepared from restenotic specimen (n=10) and, as control, from intima and media of healthy muscular arteries (n=10) were amplified using a novel polymerase chain reaction protocol and hybridized to cDNA arrays for the identification of differentially expressed genes. Expression of desmin and mammary-derived growth inhibitor was downregulated, whereas expression of FK506-binding protein 12 (FKBP12), thrombospondin-1, prostaglandin G/H synthase-1, and the 70-kDa heat shock protein B was found to be upregulated with high statistical significance in human neointima. Using immunohistochemistry, FKBP12, a negative regulator of TGF-β signaling, was also upregulated at the protein level in neointima providing a rationale for the therapeutic effect of the FKBP12 ligand rapamycin in the treatment of a porcine restenosis model.

FKBP12 is involved in controlling transforming growth factor TGF-β receptor I mediated signaling pathway. Binding of FKBP12 to the TGF-β receptor might prevent TGF-β induced cell cycle arrest. FKBP12 is also the receptor fur rapamycin, which was shown to reduce neointima formation in animal models and clinical trials in humans. Binding of rapamycin to FKBP12 might counteract ist inhibitory effect on TGF-β.

cDNA samples were prepared from neointima derived from tissue specimen from patients with symptomatic in-stent restenosis and, as a control, from the media of normal arteries. cDNA was amplified by PCR and analyzed by cDNA array technology. Strong upregulation of FKBP12-specific mRNA expression was observed in neointima and was confirmed by immunohistochemistry on human SMCs. FKBP12 was found in the cytoplasma of neointimal SMCs while no staining was observed in SMCs from control media.

Up to now the molecular mechanism for rapamycin action in neointima formation has not been known. The finding that FKBP12 which acts as a receptor for rapamycin is upregulated in restenotic tissue provides a molecular basis for the prefered action of rapamycin on neointima formation. The identification of FKBP12 as a gene with elevated expression in restenotic tissue validates the data obtained by the gene expression analysis of human restenotic tissue.

To gain further insight into transcriptional and signaling events governing smooth muscle cell migration, proliferation and synthesis of extracellular matrix, differential gene expression screening was employed using cDNA array technology with probes generated from microscopic specimen of human restenotic tissue. The power of this technology is the ability to simultaneously study in one sample the expression of thousands of genes (Kurian, (1999) J Pathol 187:267-271). A previous hurdle of using this method was the need for micrograms of mRNA or cRNA from samples usually composed of 10⁶-10⁷ cells. Here, the novel technology, as described hereinabove, was employed. This allowed the generation of representative cDNA amplificates from a single cell or a low number of cells in quantities sufficient for comprehensive cDNA array hybridization.

10 specimen of each neointimal and quiescent media for the expression of 2,435 genes of known function. While the expression of house-keeping genes was largely comparable between normal and restenotic tissue close to 10 percent of studied genes showed an increased or decreased level of expression. In the present study, it was focused on selected genes that have previously been associated with restenosis. Desmin and mammary-derived growth factor inhibitor (MDGI) expression was selectively downregulated while the expression of prostaglandin G/H synthase-1 (COX-1), thrombospondin-1 (TSP-1), heat-shock protein-70 B (hsp70B) and FK506-binding protein 12 (FKBP12) was found to be upregulated in human neointima hyperplasia. These findings were all confirmed by gene-specific PCR. To study the significance of increased gene expression in neointima, it was investigated whether increased mRNA levels find their reflection in an increased protein level. As exemplified with FKBP12 using immunohistochemistry, it was indeed found a robust overexpression of this regulator of TGF-β signaling in restenotic tissue. This study shows that cDNA array technology can be used to reliably identify differentially expressed genes in healthy and diseased human tissue even if only very small amounts of material are available.

The in-stent restenosis study group consisted of 13 patients who underwent separate atherectomy procedures by Helix cutter device artherectomy (X-sizer, Endicor) within the renarrowed stent between 4-23 month after primary stent implantation. All patients gave informed consent to the procedure and received 15,000 units heparin before the intervention followed by intravenous heparin infusion, 1,000 units/h for the first 12 h after sheat removal as standard therapy. All patients received aspirin, 500 mg intravenously, before catherisation, and postinterventional antithrombotic therapy consisted of ticlopidine (250 mg bds) and aspirin (100 mg bds) throughout the study.

Sample Preparation was carried out as follows:

Atherectomy specimen were immediately frozen in liquid nitrogen after debulking of the lesion, and kept in liquid nitrogen until mRNA preparation was performed as described. For histology and immunhistochemistry of the in-stent restenotic tissue from coronary arteries (n=3), the samples were fixed in 4% paraformaldehyd and embedded in paraffin as described.

The control group consisted of 5 specimen of muscular arteries of the gastrointestinal tract from five different patients and 5 specimen from coronary arteries from three different patients who underwent heart transplantation. The control specimen were immediately frozen in liquid nitrogen. Prior to mRNA preparation, media and intima of the control arteries were prepared and examined for atherosclerotic changes by immunhistochemistry. If there were no atherosclerotic changes of the vessel morphology, the specimen (approx. 1×1 mm) were used as healthy control samples and mRNA and cDNA preparation was performed as described.

For immunohistochemistry of FKBP12, neointima specimen of carotid restenotic arteries (n=2) were obtained by atherectomy and immediately frozen in liquid nitrogen after removal. Three 3 μm serial frozen sections of the samples were mounted onto DAKO ChemMate™ Capillary Gap Microscope slides (100 μM).

mRNA Preparation and amplified cDNA was carried out as follows:

Specimen of quiescent vessels or in-stent restenotic tissue were quick-frozen and kept in liquid nitrogen until mRNA preparation and cDNA synthesis was performed. Frozen tissue was ground in liquid nitrogen and the frozen powder dissolved in Lysis/Binding buffer (100 mM Tris-HCl, pH 7.5, 500 mM LiCl, 10 mM EDTA, pH 8.0, 1% LiDS, 5 mM dithiothreitol (DTT)) and homogenized until complete lysis was obtained. The lysate was centrifuged for 5 min at 10,000 g at 4° to remove cell debris. mRNA was prepared using the Dynbeads® mRNA Direct Kit™ (Dynal, Germany) following the manufacture's recommendation. Briefly, lysate was added to 50 μL of pre-washed Dynabeads Oligo (dT)₂₅ per sample and mRNA was annealed by rotating on a mixer for 30 min at 4° C. Supernatant was removed and Dynabeads Oligo (dT)₂₅/mRNA complex was washed twice with washing buffer containing Igepal (50 mM Tris-HCl, pH 8.0, 75 mM KCl, 10 mM DTT, 025% Igepal), and once with washing buffer containing Tween-20 (50 mM Tris-HCl, pH 8.0, 75 mM KCl, 10 mM DTT, 0.5% Tween-20).

cDNA was amplified by PCR using the procedure as described hereinabove. First-strand cDNA synthesis was performed as solid-phase cDNA synthesis. Random priming with hexanucleotide primers was used for reverse transcription reaction. mRNAs were each reversely transcribed in a 20 μL reaction volume containing 1×First Strand Buffer (Gibco), 0.01 M DTT (Gibco), 0.25% Igepal, 50 μM CFL5c-Primer [5′-(CCC)₅ GTC TAG A (NNN)₂-3′], 0.5 mM dNTPs each (MBI Fermentas) and 200 U Superscript II (Gibco), and incubated at 44° C. for 45 min. A subsequent tailing reaction was performed in a reaction volume of 10 μL containing 4 mM MgCl₂, 0.1 mM DTT, 0.2 mM dGTP, 10 mM KH₂PO₄ and 10 U of terminal deoxynucleotide transferase (MBI Fermentas). The mixture was incubated for 24 min at 37° C.

cDNA was amplified by PCR in a reaction volume of 50 μL containing 1×buffer 1 (Expand™ Long Template PCR Kit, Boehringer Mannheim), 3% deionized formamide, 1,2 μM CP2-Primer [5′-TCA GAA TTC ATG (CCC)₅-3′], 350 μM dNTP and 4.5 U DNA-Polymerase-Mix (Expand™ Long Template PCR Kit, Roche Diagnostics, Mannhein). PCR reaction was performed for 20 cycles with the following cycle parameters: 94° C. for 15 sec, 65° C. for 0:30 min, 68° C. for 2 min; for another 20 cycles with: 94° C. for 15 sec, 65° C. for 30 sec, 68° C. for 2:30+0:10/cycle min; 68° C. 7 min; 4° C. forever.

25 ng of each cDNA was labeled with Digoxigenin-11-dUTP (Dig-dUTP) (Roche Diagnostics) during PCR. PCR was performed in a 50 μL reaction with 1×Puffer 1, 120 μM CP2 primer, 3% deionized formamide, 300 μM dTTP, 350 μM dATP, 350 μM dGTP, 350 μM dCTP, 50 μM Dig-dUTP, 4.5 U DNA-Polymerase-Mix. Cycle parameters were: one cycle: 94° C. for 2 min; 15 cycles: 94° C. for 15 sec, 63° C. for 15 sec, 68° C. for 2 min; 10 cycles: 94° C. for 15 sec, 68° C. for 3 min+5 sec/cycle; one cycle: 68° C., 7 min, 4° C. forever.

Hybridization of Clontech cDNA Arrays with dUTP-labeled cDNA Probes was carried out as follows:

cDNA arrays were prehybridized in DigEASYHyb solution (Roche Diagnostics) containing 50 mg/L DNAsel (Roche Diagnostics) digested genomic E. coli DNA, 50 mg/L pBluescript plasmid DNA and 15 mg/L herring sperm DNA (Life Technologies) for 12 h at 44° C. to reduce background by blocking non-specific nucleic acid-binding sites on the membrane. Hybridization solution was hybridized to commercially available cDNA arrays with selected genes relevant for cancer, cardiovascular and stress response (Clontech). Each cDNA template was denatured and added to the prehybridization solution at a concentration of 5 μg/ml Dig-dUTP-labeled cDNA. Hybridization was performed for 48 hours at 44° C.

Blots were subsequently rinsed once in 2×SSC/0.1% SDS and once in 1×SSC/0.1% SDS at 68° C. followed by washing for 15 min once in 0.5×SSC/0.1% SDS and twice for 30 min in 0.1×SSC/0.1% SDS at 68° C. For detection of Dig-labeled cDNA hybridized to the array, the Dig Luminescent Detection Kit (Boehringer, Mannheim) was used as described in the user manual. For detection of the chemiluminescence signal, arrays were exposed to chemiluminescence films for 30 min at room temperature. Quantification of array data was performed by scanning of the films and analysis with array vision software (Imaging Research Inc., St. Catharines, Canada). Background was subtracted and signals were normalized to the nine housekeeping genes present on each filter, whereby the average of the housekeeping gene expression signals was set to 1 and the background set to 0. In a pilot study, six clones enriched in one of the two probes were further analyzed by RT-PCR.

Results of the experimental studies are reported as mean expression values of the ten examined specimen of the study or control group. Differences between the two patient groups were analyzed by Wilcoxon-test (SPSS version 8.0). A p-value less than 0.03 was regarded as significant.

A selection of differential hybridization signals were confirmed by PCR using gene-specific primers. PCR reactions were performed using 2.5 ng of each cDNA in 25 μl reaction containing 1×PCR buffer (Sigma), 200 μM dNTPs, 0.1 μM of each primer and 0.75 U Taq Polymerase (Sigma). The following primers were used: desmin, 5′-ACG ATT CCC TGA TGA GGC AG-3′ and 5′-CCA TCT TCA CGT TGA GCA GG-3′; thrombospondin-1, 5′-CTG AGA CGC CAT CTG TAG GCG GTG -3′ and 5′-GTC TTT GGC TAC CAG TCC AGC AGC-5′; mammary-derived growth inhibitor, 5′-AAG AGA CCA CAC TTG TGC GG-3′ and 5′-AAT GTG GTG CTG AGT CGA GG-5′; prostaglandin G/H synthase-1, 5′-CGG TGT CCA GTT CCA ATA CC-3′ and 5′-CCC CAT AGT CCA CCA ACA TG-3′; FKBP12, 5′-ATG CCA CTC TCG TCT TCG AT-3′ and 5′-GGA ACA TCA GGA AAA GCT CC-3′; heat shock protein 70B, 5′-TAC AAG GCT GAG GAT GAG GC-3′ and 5′-CTT CCC GAC ACT TGT CTT GC-3′, and β-actin, 5′-CTA CGT CGC CCT GGA CTT CGA GC-5′ and 5′-GAT GGA GCC GCC GAT CCA CAC GG-3′. PCR products were subjected to electrophoresis on a 2% agarose gel containing ethidium bromide (0.5 μg/ml agarose solution) in TAE buffer (20 mM Tris/HCl, 10 mM acetic acid, 1 mM EDTA).

Immunohistochemistry was carried out as follows:

Immunohistochemistry for cell typing was performed on paraffin-embedded sections of three neointima specimen from coronary in-stent restenosis and, for detection of FKBP12, on frozen sections of four neointima specimen from carotid restenosis. Three μm serial sections were mounted onto DAKO ChemMate™ Capillary Gap Microscope slides (100 μm) baked at 65° C. overnight, deparaffinized and dehydrated according to standard protocols. For antigen retrieval, specimens were boiled 4 min in a pressure cooker in citrate buffer (10 mM, pH 6.0). Endogenous peroxidase was blocked by 1% H₂O₂/methanol for 15 minutes. Unspecific binding of the primary antibody was reduced by preincubation of the slides with 4% dried skim milk in Antibody Diluent (DAKO, Denmark). Immunostaining was performed by the streptavidin-peroxidase technique using the ChemMate Detection Kit HRP/Red Rabbit/Mouse (DAKO, Denmark) according to the manufacturer's description. The procedures were carried out in a DAKO TechMate™ 500 Plus automated staining system. Primary antibodies against smooth muscle actin (M0635, DAKO, Denmark; 1:300), CD3 (A0452, DAKO, Denmark; 1:80), MAC387 (E026, Camon, Germany; 1:20) and FKBP12 (SA-218, Biomol, Germany, 1:20) were diluted in Antibody Diluent and incubated for 1 h at room temperature. After nuclear counterstaining with hematoxylin, the slides were dehydrated and coverslipped with Pertex (Medite, Germany).

For FKBP12 immunhistochemistry, 3 μm frozen, serial sections of the neointima specimen from carotid restnosis were mounted onto DAKO ChemMate™ Capillary Gap Microscope slides (100 μm).

The following results were obtained:

(a) The Cellular Composition of Debulked In-stent Restenotic Material

Representative samples obtained from x-sizer treatment of a neointimal hyperplasia were analyzed by immunhistochemistry in order to determine its cellular composition. The restenotic tissue analyzed was removed by x-sizer debulking from coronary arteries more than two month after PTCA and stent implantation. The amount of tissue generated by this procedure was very low containing an estimated 300-10000 cells. FIG. 7A shows an E.-van-Giesson staining of a section cut from a small sample of debulked restenotic material. With this staining procedure, collagen fibers stain red, fibrin stains yellow and cytoplasm of smooth muscle cells stains dark-yellow-brown. The majority of the volume of debulked material was composed of loose extracellular matrix-like collagen fibers stained in light red. Yellow fibrin staining was barely detectable. Cells with spindle-shaped nuclei and a yellow/brown-stained cytoplasm were frequent. Their identity as smooth muscle cells and their high abundance in restenotic material was supported by immunostaining with an antibody against smooth muscle α-actin (FIG. 7B). There, the staining pattern of a section from an entire specimen as used for gene expression analysis is shown. As described below, such samples also gave raise to a strong smooth muscle-specific α-actin mRNA signal (see FIG. 8). These results support findings from previous studies (Komatsu, (1998), Circulation 98:224-233; Strauss (1992), J. Am. Coll. Cardiol. 20:1465-1473; Kearney (1997), Circulation 95:1998-2002) demonstrating that the main cell type found in neointima is derived from smooth muscle cells. As described in the literature, mononuclear infiltrates in some areas of debulked restenotic tissue specimen could also be identified (data not shown). These infiltrates consisted mainly of macrophages and to a lesser degree of t-lymphocytes. No b-lymphocytes were detectable in the restenotic tissue by using an antibody against CD20 for immunhistochemical staining (data not shown).

(b) Expression of Specific Genes in Microscopic Human Tissue Samples

In order to optimally preserve the in situ mRNA levels, restenotic and control specimen were immediately frozen after harvest in liquid nitrogen and carefully lyzed as described hereinabove. After PCR amplification of the synthesized cDNA the amount of the amplified cDNA was measured by a dot blot assay and found to be between 200-300 ng/μl. The quality of every amplified cDNA sample was tested by gene-specific PCR using primers detecting cDNAs for β-actin, smooth muscle cell α-actin and the ubiquitous elongation factor EF-1α. FIG. 8 shows a representative result with material from patient B and control media from donor b. In both specimen, PCR signals of the correct size from house-keeping genes β-actin and EF-1α were detectable in equivalent amounts (compare lanes 1 and 2 with lanes 4 and 5). Additionally, α-actin signals as marker for smooth muscle cells was obtained from each specimen (lanes 3 and 6). These results show that mRNA preparation, cDNA synthesis and PCR amplification of cDNA is feasible with microscopic human restenosis samples.

(c) Comparative Gene Expression Profiling Using Microscopic Human Tissue Samples

To identify differentially expressed mRNAs in restenotic versus healthy specimen, the cDNAs was labeled during PCR amplification with digoxigenin-labeled dUTP as described hereinabove. This label allows for a highly sensitive, chemiluminescence-based detection of hybridization signals of cDNA arrays on photographic films. The nylon filters with cDNA arrays were pre-hybridized with DNAsel-digested genomic E. coli DNA and with DNAsel-digested pBluescript plasmid DNA. This procedure was employed to maximally reduce non-specific DNA binding to the array. Each labeled probe was hybridized to three different commercial cDNA arrays which allowed for the expression analysis of a total of 2,435 known genes. FIG. 9 shows a representative hybridization pattern obtained with one array using probes prepared from restenotic tissue of patient B (panel A) and media of donor b (panel B). Consistent with the gene-specific analysis shown in FIG. 8, comparable hybridization signals were obtained with the positive control of human genomic cDNA spotted on the right and bottom lanes of the array and with cDNA spots of various housekeeping genes (see for instance, spots D). If a biological specimen was omitted from cDNA synthesis and PCR amplification reactions almost no hybridization signals were obtained (FIG. 9, panel C), showing that hybridization signals were almost exclusively derived from added samples and not from DNA contaminations in reagents or materials used.

Visual inspection of the hybridization patterns readily identified a number of signals that are different between healthy and diseased tissue (for instance signals A, B and C in FIGS. 9A and B). Samples from restenotic tissues consistently gave more signals than control tissues. Hybridization signals obtained from the use of three different cDNA arrays with 10 restenosis patient samples and 10 normal media samples were quantitated by densitometric analysis of photographic films and the data electronically compiled and further analyzed for statistics. Expression levels for 53 out of 2,435 genes is shown in FIG. 10 whereby one grey value corresponds to the signal intensity as shown in the figure legend. A considerable variation of gene expression is evident for most genes shown which may reflect genetic and physiological differences of patients and donors. For further analysis and verification by gene-specific PCR, only genes were considered that showed a differential expression with a statistical difference of at least p=0.03 by the Wilcoxon Test. Six such genes are highlighted in the list (FIG. 10). A total of 224 genes out of 2435 known genes was found to be differentially regulated in neointima with high statistical significance. Their comprehensive in-depth analysis will be published elsewhere. Indicative for a comparable sample quality, eight housekeeping genes showed very similar hybridization signal intensities with all 20 samples (FIG. 10, bottom).

(d) Validation of cDNA Array Data by Gene-specific PCR

Out of the list depicted in FIG. 10, six differentially regulated genes and one housekeeping gene were selected for validation of hybridization signals through PCR using gene-specific primers. All PCR signals obtained had the predicted size. In support of an equal quality of samples, the B-actin signal (bottom) showed a very similar intensity with all 20 samples. By comparing gene-specific PCR signals (FIG. 11) with hybridization signals obtained from cDNA arrays (FIG. 11) it was found that 135 out of 140 signals matched with respect to intensity. This corresponds to a 96% fidelity of hybridization signals from cDNA arrays showing that the here employed gene expression profiling approach is comparable with respect to quality and sensitivity to gene-specific PCR.

(e) Aberrant Gene Expression in Human Restenotic Tissue

Desmin, a mesenchymal marker, was found strongly expressed in the control media, whereas only weak signals were found in the restenotic specimen (FIGS. 10 and 11). Desmin is a marker for SMCs that is highly expressed in quiescent, differentiated SMCs. Its expression is reduced in de-differentiated, proliferating SMCs, e.g., in SMCs of atherosclerotic plaques (Ueda (1991), Circulation 83:1327-1332). Downregulation of desmin in restenotic tissue implies that the spindle-shaped cells in the restenotic material are de-differentiated, proliferating SMCs. Inversely, TSP-1, an extracellular matrix protein, that is important in TGF-β activation and SMC migration and proliferation (Yehualaeshet (1999), Am J Pathol 155:841-851; Scott (1988), Biochem.Biophys.Res.Commun. 150:278-286), is markedly upregulated in the majority of neointimal specimen versus the control samples. The COX-1, stress-induced hsp70B and the ubiquitously expressed FKBP12 genes were significantly upregulated in almost all neointimal hyperplasia and barely, if at all, expressed in control specimen (FIGS. 10 and 11). The tumor suppressor MDGI was strongly expressed in quiescent smooth muscle whereas little expression was found in a few neointima hyperplasia samples.

None of the restenotic lesions expressed desmin (0/0) compared to 100% of controls (10/10), only 30% (3/10) of the neointimal specimen expressed MDGI very slightly, whereas it was highly expressed in 8/10 (80%) of the controls. Otherwise, TSP-1 (7/10), COX-1 (9/10), hsp70B (8/10) and FKBP12 (10/10) were significantly upregulated in neointimal versus control specimen (TSP-1 [0/10], hsp70B [0/10], COX-1 [0/10], FKBP 12 [1 /10]).

(f) FKBP12 Protein Expression Is Upregulated in Human Restenotic Tissue

Upregulation of mRNA levels does not stringently indicate an increased level of protein. Among the genes that were found to be upregulated in human neointima, FKBP12 is particularly interesting since it is a regulator of TGF-β signaling and target for the drugs FK506 and rapamycin. A therapeutic effect of rapamycin in rodent models (Gallo (1999), Circulation 99:2164-2170) of restenosis is poorly understood but may be related to changes in the expression level of FKBP12. Using an antibody specific for FKBP12, human restenotic tissue from carotid restenosis (n=3) was analyzed and control tissue (n=3) for the expression of the protein. As shown in FIG. 12, an increase in FKBP12 protein in the cytoplasm of SMCs from restenotic lesions as identified by their spindle-shaped nuclei was detected (FIGS. 12B and D). Whereas no FKBP12 was detectable in control SMCs of healthy media (FIG. 12C), a distinct staining in SMCs of neointima was found (FIG. 12D). Interestingly, especially smooth muscle cells lying in the border zone between neointima and healthy media of restenotic vessels expressed high levels of the FKBP12 protein (FIG. 11B).

### EXAMPLE VI

### Characterization of the Transcriptome of Human Restenotic Tissue

The expression of 2,435 genes of known function (see Example V) was investigated in atherectomy specimen of 10 patients with in-stent restenosis, blood cells of 10 patients, normal coronary artery specimen of 11 donors, and cultured human coronary artery smooth muscle cells. 224 genes that were differentially expressed with high statistical significance (p<0.03) between neointima and control tissue which could be grouped as follows: (1) genes only expressed in neointima; (2) genes expressed in both neointima and proliferating smooth muscle cells; (3) genes expressed in both neointima and blood samples; and (4) genes expressed in control tissue but barely in neointima. The transcriptome of human neointima showed significant changes related to proliferation, apoptosis, inflammation, cytoskeletal reorganization and tissue remodeling. Furthermore, in neointima 32 upregulated genes were identified that are related to interferon-γ signaling.

In the present study, 10 specimen of neointimal and 11 specimen of quiescent intima/media for the expression of 2,435 human genes of known function were analyzed. While the expression of housekeeping genes was largely comparable between normal and restenotic tissue, an impressive number of genes (n=224) showed an increased or decreased level of expression. The gene expression pattern in neointima showed the anticipated proliferative response with induction of genes mainly expressed in G1/S phase, changes of the smooth muscle phenotype from contractile to synthetic SMCs and changes in synthesis of extracellular matrix proteins. Additionally, a pro-inflammatory expression pattern characterized by the presence of markers for macrophages and T lymphocytes and by the expression of numerous genes with known functions in the cellular response to IFN-γ were observed. The IRF-1 protein, a pivotal transcription factor in IFN-γ signaling, was found overexpressed in SMCs of human neointima.

The clinical characteristics of the patients of the study group of this Example are presented in Table 7.

TABLE 7
Clinical Data of 13 Patients

| | | | | | Interval/ | | | | | | | |
|---|---|---|---|---|---|---|---|---|---|---|---|---|
| | | | | | Stent/ | Interval | | | Arterial | | Multi- | |
| | | | Indication | Stent | Poststent | Stent/ | | Hypercholes- | Hyper- | Diabetes | vessel | familial |
| Patient | Age, y | Sex | for Stent | Site | Restenosis | Debulking | Smoker | terolemia | tension | mellitus | disease | risk |
| 1 | 77 | m | AMI | RCA | 5 m | 11 m | − | + | + | + | + | − |
| 2 | 62 | m | SAP | LAD | 6 m | 19 m | − | + | + | − | − | + |
| 3 | 57 | m | ISAP | ACVB 7 | 4 m | 10 m | + | − | + | + | + | − |
| 4 | 68 | m | failed Bypass | ACVB 14 | 4 m | 4 m | − | + | + | − | + | − |
| 5 | 80 | m | AMI | LAD | 7 m | 7 m | − | + | + | − | + | − |
| 6 | 67 | m | Restenosis | RCA | 12 m | 23 m | − | + | + | − | + | − |
| 7 | 44 | f | Restenosis after PTCA | RCA | 3 m | 8 m | − | + | 7 | − | − | − |
| 8 | 75 | m | Restenosis after PTCA | RCA | 6 m | 6 m | − | + | − | − | + | − |
| 9 | 86 | m | Restenosis after PTCA | RCA | 5 m | 5 m | − | + | + | − | − | − |
| 10 | 44 | m | Restenosis after PTCA | LAD | 6 m | 6 m | + | + | + | − | − | + |
| 11 | 76 | m | AMI | LAD | 6 m | 6 m | − | + | + | − | − | − |
| 12 | 46 | m | Restenosis after PTCA | LAD | 5 m | 5 m | − | + | + | − | − | + |
| 13 | 69 | m | Restenosis after PTCA | LAD | 4 m | 16 m | − | + | + | − | + | − |

All atherectomy specimen were immediately frozen in liquid nitrogen after debulking of the lesion, and kept in liquid nitrogen until mRNA preparation was performed as described above.

The control group consisted of 5 specimen of muscular arteries of the intestine from five patients and 6 specimen from coronary arteries from three patients who underwent heart transplantation. The control specimen were immediately frozen in liquid nitrogen. Prior to mRNA preparation, media and intima of the arteries were prepared. A small piece of the specimen (approx. 1 mm³) was immediately lysed, whereas the rest was histologically examined for atherosclerotic changes. If there were no atherosclerotic changes of vessel morphology detectable, the specimen were used as “healthy” control samples and mRNA and cDNA preparation was performed as described.

The neointimal tissue of carotid (n=3) and femoralis (n=3) arteries was generated by atherectomy within the restenosis and immediately frozen after removal in liquid nitrogen. For histologic evaluation and immunohistochemistry of the in-stent restenotic tissue from coronary arteries (n=3) and of the neointima of restenotic peripheral arteries (n=6), the samples were fixed in 4% paraformaldehyd and embedded in paraffin as described.

Blood samples were obtained immediately after revascularization of the restenotic vessel. Eight ml blood samples were collected into 35 ml of TriReagent Blood (MBI Fermentas, Germany) and subsequently frozen at −80° C. until RNA preparation was performed as described in the manufacture's protocol. 1 μg of total RNA of blood cells were dissolved in 1000 μL Lysis/Binding buffer and mRNA and cDNA synthesis was prepared as described above.

Cell Culture Was carried out as follows:

Primary human coronary artery smooth muscle cells (CASMCs) were obtained from CellSystems (St. Kathrinen, Germany) and were grown in Smooth Muscle Cell Growth Medium (CellSystems, St. Kathrinen, Germany) containing 5% fetal calf serum (CellSystems, St. Kathrinen, Germany) at 37° C. in a humidified atmosphere of 5% CO₂. CASMCs were used in experiments between passages 2 and 4. For cDNA synthesis of proliferating CASMCs were washed three times with ice-cold phosphate-buffered saline and 1×10⁴ cells were subsequently lysed in 1000 μL Lysis/Binding puffer before mRNA was prepared as described above.

Determination of Gene Expression Patterns was carried out as follows:

Sample mRNA preparation, cDNA synthesis, PCR amplification and probe labeling, cDNA array hybridization and data analysis were performed as described hereinabove, in particular in Example V. The obtained cDNA probes were hybridized to Human 1.2, Cancer 1.2, Cardiovascular and Stress cDNA arrays (Clontech, Heidelberg, Germany) with a total of 2,435 genes of known function. There was an approximately 20% redundancy of genes among cDNA arrays. For analysis of microscopic human tissue samples down to a single cell level the here described new method of cDNA synthesis and PCR amplification was used (see Examples I to V).

Quantification of array data was performed by scanning of the films and analysis with array vision software (Imaging Research Inc., St. Catharines, Canada). Background was subtracted and signals were normalized to the nine housekeeping genes present on each filter, whereby the average of the housekeeping gene expression signals was set to 1 and the background set to 0. For the logarithmic presentation shown in FIG. 1, values were multiplied by 1000. A mean value ≧0,05 in the average of all samples in one group was regarded as a positive signal. Differences in the mean expression level by a factor ≧2.5-fold between the study and the control group were further statistically analyzed.

Results of the experimental analysis are given as mean expression values of the ten examined specimen of the study group or the eleven examined specimen of the control group. Differences between the patient and donor groups were analyzed by the Wilcoxon-test (SPSS version 8.0). Genes were only considered to be differentially expressed between the two groups if their p-values in the Wilcoxon test were <0.03, and if a differential expression was observed in at least 5 out of 10 samples within one study group, while there was 0 out of 10 within the other group; or at least 7 out of 10 samples within one group, while there were maximally 3 out of 10 within the other group.

Immunhistochemistry was carried out as follows:

Immunhistochemistry was performed on paraffin-embedded sections from 3 neointima specimen from coronary in-stent restenosis, 3 neointima specimen from A. femoralis and 3 neointima specimen from carotid neointima specimen. Three μm serial sections were mounted onto DAKO ChemMate™ Capillary Gap Microscope slides (100 μm), baked at 65° C. overnight, deparaffinized and dehydrated according to routine protocols. For antigen retrieval, specimen were boiled 4 minutes in a pressure cooker in citrate buffer (10 mMol, pH 6.0). Endogenous peroxidase was blocked by 1% H₂O₂/methanol for 15 minutes. Unspecific binding of the primary antibodies was reduced by preincubation of the slides with 4% dried skim milk in Antibody Diluent (DAKO, Denmark). Immunostaining was performed by the streptavidin-peroxidase technique using the Dako ChemMate Detection Kit HRP/Red Rabbit/Mouse (DAKO Denmark) according to the manufacturers description. The procedures were carried out in a DAKO TechMate™ 500 plus automated staining system. Primary antibodies against smooth muscle actin (M0635, DAKO, Denmark; 1:300), CD3 (A0452, DAKO, Denmark; 1:80), MAC387 (E026, Camon, Germany; 1:20) and IRF-1 (sc-497, Santa Cruz, U.S.A.) were diluted in Antibody Diluent and incubated for 1 h at room temperature. After nuclear counterstaining with hematoxylin, slides were dehydrated and coverslipped with Pertex (Medite, Germany).

The following results were obtained:

(a) Differential Gene Expression in Human Neointima

A total of 1,186 genes (48.7%) out of 2,435 yielded detectable hybridization signals on cDNA arrays with neointima and control samples over a 20-fold range of expression level (FIG. 13A) Thereof 352 genes (14.5%) appeared to be differentially expressed by a factor >2.5-fold between restenotic and control samples. However, expression levels considerably varied among individual samples (see, e.g., FIG. 15). Therefore, a statistical analysis was employed to identify those genes that are differentially expressed between study and control groups with high significance (see Methods). This way, 224 genes (9.6%) were identified that were differentially expressed by a factor of at least 2.5-fold between the restenosis study group and the control group with a significance in the Wilcoxon test of p<0.03. 167 (75%) genes thereof were found overexpressed and 56 genes (25%) underexpressed in the restenosis study group compared to the control group (FIG. 13B).

In addition to the statistical significance, the validity of expression data was supported by a 20% redundancy of cDNA elements on the four arrays used. This way, a substantial number of hybridization signals was determined in duplicate or triplicate in independent hybridization experiments. Four examples of duplicate determinations are shown in FIG. 16 (top) which all showed a high degree of reproducibility. As a further validation of hybridization signals, 38 of the differentially expressed genes were selected for PCR analysis of cDNA samples using gene-specific primers. Hybridization signals for 35 (92%) out of 38 genes could be verified by gene-specific PCR yielding signals of the predicted size and relative quantity (data not shown). These data shows that the employed cDNA array approach is comparable with respect to quality and sensitivity to gene-specific PCR. Lastly, among the 224 aberrantly expressed genes in neointima 112 have previously been described in the literature as being expressed in neointima, SMCs, fibroblasts, endothelial cells or mesenchym (FIG. 14 marked with ‘#’).

With respect to neointima expression, the 224 aberrantly regulated genes fell into four subgroups (FIG. 14). Group I lists 62 genes that were overexpressed in neointima and not highly or detectably expressed in control vessels, CASMCs or blood cells (FIG. 14A). In group II, 43 genes are listed that are similarly expressed in neointima and CASMCs, suggesting that this gene cluster in neointima was contributed by proliferating SMCs (FIG. 14B). In group III, 62 genes are listed that are similarly expressed in neointima and blood cells, suggesting that this gene cluster was contributed to that of neointima by infiltrated blood cells (FIG. 14C). This notion is supported by the expression in group III of the largest number of genes related to inflammation in all four groups. Lastly, in group IV, 56 genes are listed that are downregulated in neointima compared to the control group (FIG. 14D). In the following, the aberrant expression of selected genes in neointima will be discussed in the context of gene function.

In summary, the following differentially expressed genes have been detected in human neointima:

| | GenBank | SwissProt |
|---|---|---|
| Gene Name | Accession # | Accession # |
| 80-kDa nuclear cap-binding protein | D32002 | Q09161 |
| activator 1 140-kDa subunit (A1 140-kDa subunit); replication factor C large subunit; | L14922 | P35251 |
| DNA-binding protein PO-GA | | |
| activator 1 37-kDa subunit; replication factor C 37-kDa subunit (RFC37); RFC4 | M87339 | P35249 |
| adenylate kinase isoenzyme 1 (AK1); ATP-AMP transphosphorylase; myokinase | J04809 | P00568 |
| adipocyte fatty acid-binding protein 4 (FABP4; AFABP); adipocyte lipid-binding protein (ALBP) | J02874 | P15090 |
| allograft inflammatory factor 1 (AIF1); ionized calcium-binding adapter molecule 1 | U19713 | P55008 |
| alpha-1-antitrypsin precursor; alpha-1 protease inhibitor; alpha-1-antiproteinase | X02920 | P01009 |
| alpha-2-antiplasmin | D00174 | P08697 |
| alpha-2-macroglobulin precursor (alpha-2-M) | M11313 | P01023 |
| alpha-galactosidase A precursor; melibiase; alpha-D-galactoside galactohydrolase | X05790 | P06280 |
| amiloride-sensitive epithelial sodium channel beta subunit; nonvoltage-gated sodium channel 1 beta subunit | X87159 | P51168 |
| (SCNEB; beta NACH); SCNN1B | | |
| angiotensinogen precursor (AGT) | K02215 | P01019 |
| apolipoprotein E precursor (APOE) | M12529 | P02649 |
| atrial natriuretic peptide receptor B precursor (ANPB; NPRB); guanylate cyclase B (GCB) | L13436 | P20594 |
| autosomal dominant polycystic kidney disease II (PKD2) | U50928 | Q13563 |
| B-cell-associated molecule CD40 | X60592 | P25942 |
| BCL-2 binding athanogene-1 (BAG-1); glucocorticoid receptor-associated protein RAP46 | S83171; Z35491 | Q99933 |
| BCL-2-related protein A1 (BCL2A1); BFL1 protein; hemopoietic-specific early response protein; | U29680; Y09397 | Q16548; Q99524 |
| GRS protein | | |
| BIGH3 | M77349 | Q15582 |
| bikunin; hepatocyte growth factor activator inhibitor 2 | U78095 | O00271; O43291 |
| brain glucose transporter 3 (GTR3) | M20681 | P11169 |
| brain-specific polypeptide PEP-19; brain-specific antigen PCP-4 | U52969 | P48539 |
| Bruton's tyrosine kinase (BTK); agammaglobulinaemia tyrosine kinase (ATK); | U10087; X58957 | Q06187 |
| B-cell progenitor kinase (BPK) | | |
| C5a anaphylatoxin receptor (C5AR); CD88 antigen | M62505 | P21730 |
| cadherin 16 (CDH16); KSP-cadherin | AF016272 | P75309 |
| calcium & integrin-binding protein (CIB) | U85611 | Q99828 |
| carboxypeptidase H precursor (CPH); carboxypeptidase E (CPE); enkephalin convertase; prohormone | X51405 | P16870 |
| processing carboxypeptidase | | |
| carboxypeptidase N | X14329 | P15169 |
| caspase-8 precursor (CASP8); ICE-like apoptotic protease 5 (ICE-LAP5); | U60520; U58143; | Q14790; Q15780 |
| MORT1-associated CED-3 homolog (MACH); FADD-homologous ICE/CED-3-like | X98172; AF00962 | |
| protease (FADD-like ICE; FLICE); apoptotic cysteine protease MCH-5 | | |
| caveolin 3 | AF043101 | P56539 |
| CBL-B | U26710 | Q13191 |
| CDC42 homolog; G25K GTP-binding protein (brain isoform + placental isoform) | M35543 + M57298 | P21181 + P25763 |
| cell surface adhesion glycoproteins LFA-1/CR3/p150, 95 beta-subunit precursor; LYAM1; integrin beta 2 | M15395 | P05107; Q16418 |
| (ITGB2); CD18 antigen; complement receptor C3 beta subunit | | |
| cell surface glycoprotein mac-1 alpha subunit precursor; CD11B antigen; leukocyte adhesion receptor MO1; | J04145 | P11215 |
| integrin alpha M (ITGAM); neutrophil adherence receptor alpha M subunit; CR3A | | |
| cell surface glycoprotein MUC18; melanoma-associated antigen A32; CD146 antigen; melanoma adhesion | M28882 | P43121 |
| molecule | | |
| C-fgr proto-oncogene (p55-FGR); SRC2 | M19722 | P09769 |
| c-fos proto-oncogene; G0S7 protein | K00650 | P01100 |
| chemokine receptor-like 2; IL8-related receptor DRY12; flow-induced endothelial G protein-coupled receptor | AF015257 | Q99527; |
| (FEG1); G protein-coupled receptor GPR30; GPCR-BR) | | Q99981; |
| | | O00143; Q13631 |
| clone 23815 (Soares library 1NIB from IMAGE consortium) | U90916 | none |
| coagulation factor XII | M11723 | P00748 |
| collagen 16 alpha 1 subunit precursor (COL16A1) | M92642 | Q07092 |
| collagen 18 alpha 1 subunit (COL18A1) | L22548 | P39060 |
| collagen 6 alpha 1 subunit (COL6A1) | X15880 | P12109 |
| collagen 6 alpha 2 subunit (COL6A2) | M34570 | Q13909; Q13911 |
| coronin-like protein P57 | D44497 | P31146 |
| c-src kinase (CSK); protein-tyrosine kinase cyl | X59932 | P41240 |
| cyclin-dependent kinase 4 inhibitor (CDK4I; CDKN2); p16-INK4; multiple tumor suppressor 1 (MTS1) | L27211 | P42771; Q15191 |
| cyclin-dependent kinase inhibitor 1 (CDKN1A); melanoma differentiation-associated | U09579; L25610 | P38936 |
| protein 6 (MDA6); CDK-interacting protein 1 (CIP1); WAF1 | | |
| cytidine deaminase (CDA) | L27943 | P32320 |
| death-associated protein 1 (DAP1) | X76105 | P51397 |
| desmin (DES) | U59167 | P17661; Q15787 |
| DNAX activation protein 12 | AF019562 | O43914 |
| dual-specificty A-kinase anchoring protein 1 | X97335 | Q92667 |
| early growth response protein 1 (hEGR1); transcription factor ETR103; KROX24; zinc finger protein 225; | X52541; M62829 | P18146 |
| AT225 | | |
| early response protein NAK1; TR3 orphan receptor | L13740 | P22736 |
| endothelial differentiation gene 1 (EDG1) | M31210; | P21453 |
| | AF022137 | |
| endothelin 2 (ET2) | M65199 | P20800 |
| ephrin A receptor 4 precursor; tyrosine-protein kinase receptor sek; hek8 | L36645 | P54764 |
| epithelial discoidin domain receptor 1 precursor (EDDR1; DDR1); | | X74979 |
| cell adhesion kinase (CAK); TRKE; RTK6 | | |
| estradiol 17 beta-dehydrogenase 1 | M36263 | P14061 |
| estrogen-related receptor alpha | X51416; Y00290 | P11474 |
| ets domain protein elk-3; NET; SRF accessory protein 2 (SAP2) | Z36715 | P41970 |
| extracellular superoxide dismutase precursor (EC-SOD; SOD3) | J02947 | P08294 |
| farnesyltransferase beta | L10414 | P49356 |
| FC-epsilon-receptor gamma subunit | M33195 | P30273 |
| FK506-binding protein (FKBP; FKBP12); peptidyl-prolyl cis-trans isomerase (PPIASE); rotamase | M34539; M80199; M80706; | |
| | M92423; J05340; X55741; X52220 | |
| fli-1 oncogene; ergB transcription factor | M93255 | Q01543 |
| FMLP-related receptor I (FMLPRII); RMLP-related receptor I (RMLPRI) | M76673 | P25089 |
| focal adhesion kinase 2 (FADK2; FAK2); cell adhesion kinase beta (CAKbeta); | L49207 + U43522 + | Q14289; |
| proline-rich tyrosine kinase 2 (PYK2) | U33284 | Q16709; Q13475 |
| frizzled-related FrzB (FRITZ) + FrzB precursor + frezzled (FRE) | U91903 + | O00181 + |
| G protein-coupled receptor EDG4 | U24163 + U68057 | Q92765 + Q99686 |
| | AF011466 | O43431 |
| G1/S-specific cyclin D1 (CCND1); cyclin PRAD1; bcl-1 oncogene | X59798; M64349 | P24385 |
| G1/S-specific cyclin D3 (CCND3) | M92287 | P30281 |
| gamma-interferon-inducible protein; IP-30 | J03909 | P13284 |
| GAP junction alpha-1 protein | X52947 | P17302 |
| glutathione-S-transferase (GST) homolog | U90313 | P78417 |
| glycerol kinase | L13943 | P32189 |
| G-protein-coupled receptor HM74 | D10923 | P49019 |
| granulocyte colony stimulating factor receptor precursor (GCSF-R); CD114 antigen | M59818 | Q99062 |
| granulocyte-macrophage colony-stimulating factor receptor alpha (GM-CSFR-alpha); CSW116 antigen | X17648 | P15509 |
| growth arrest & DNA-damage-inducible protein 45 beta (GADD45 beta) | AF078077 | none |
| growth arrest & DNA-damage-inducible protein 45 gamma (GADD45 gamma) | AF078078 | none |
| growth factor receptor-bound protein 2 (GRB2) isoform; GRB3-3; SH2/SH3 adaptor GRB2; | L29511; M96995 | P29354 |
| ASH protein + epidermal | | |
| growth factor receptor-bound protein 2 (EGFRBP-GRB2) | | |
| growth inhibitory factor; metallothionein-III (MT-III) | D13365; M93311 | P25713 |
| GTP-binding protein ras associated with diabetes (RAD1) | L24564 | P55042 |
| guanine nucleotide-binding protein G(Y) alpha 11 subunit (GNA11; GA11) | M69013 | P29992; |
| | | Q14350; O15109 |
| heart fatty acid-binding protein 3 (FABP3; HFABP); muscle fatty acid-binding protein (MFABP); mammary- | Y10255 | P05413; Q99957 |
| derived growth inhibitor (MDGI) | | |
| heat shock 70-kDa protein 6 (heat shock 70-kDa protein B) | X51757; M11236 | P48741 |
| heat shock cognate 71-kDa protein | Y00371 | P11142 |
| heme oxygenase 1 (HO1); HSOXYGR | X06985 | P09601 |
| high mobility group protein (HMG-I) | M23619 | P17096 |
| high-affinity interleukin-8 receptor A (IL-8R A); IL-8 receptor type 1; CDW128 | M68932 | P25024 |
| high-affinity nerve growth factor receptor precursor; trk-1 transforming tyrosine kinase protein; p140-TRKA; | X03541 | P04629 |
| p68-trk-T3 oncoprotein | | |
| histone H4 | X67081 | none |
| HLA class II histocompability antigen alpha subunit precursor (MHC-alpha) | M31525 | P06340 |
| homeobox protein HOXB7; HOX2C; HHO.c1 | M16937 | P09629 |
| hormone-sensitive lipase | | Q05469 |
| hydroxyacyl-CoA dehydrogenase; 3-ketoacyl-CoA thiolase; enoyl-CoA hydratase beta subunit | D16481 | P55084 |
| lgG receptor FC large subunit P51 precursor (FCRN); neonatal FC receptor; lgG FC fragment receptor | U12255 | P55899 |
| transporter alpha chain | | |
| IMP dehydrogenase 1 | J05272 | P20839 |
| insulin receptor precursor (INSR) | M10051; X02160 | P06213 |
| insulin-like growth factor binding protein 6 precursor (IGF-binding protein 6; IGFBP6; IBP6) | M62402 | P24592 |
| insulin-like growth factor I receptor (IGF1R) | X04434; M24599 | P08069 |
| integrin alpha 3 (ITGA3); galactoprotein B3 (GAPB3); VLA3 alpha subunit; CD49C antigen | M59911 | P26006 |
| integrin alpha 7B precursor (IGA7B) | X74295 | Q13683 |
| integrin alpha 8 (ITGA8) | L36531 | P53708 |
| integrin beta 7 precursor (ITGB7) | M62880; S80335 | P26010 |
| inter-alpha-trypsin inhibitor heavy chain H4 precursor (ITI heavy chain H4); plasma kallikrein-sensitive | D38595 | Q14624 |
| glycoprotein 120 (PK-120) | | |
| intercellular adhesion molecule 2 precursor (ICAM2); CD102 antigen | X15606 | P13598 |
| intercellular adhesion molecule 3 precursor (ICAM3); CDW50 antigen; ICAM-R | X69711; X69819 | P32942 |
| intercellular adhesion molecule-1 precursor (ICAM1); major group rhinovirus receptor; CD54 antigen | J03132 | P05362 |
| interferon regulatory factor 1 (IRF1) | X14454 | P10914 |
| interferon regulatory factor 7 (IRF-7) | U73036 | Q92985 |
| interferon-gamma (IFN-gamma) receptor beta subunit precursor; IFN-gamma accessory factor 1 (AF1); IFN- | U05875 | P38484 |
| gamma transducer 1 (IFNGT1) | | |
| interferon-gamma receptor (IFNGR) | A09781 | none |
| interferon-induced 56-kDa protein (IFI-56K) | X03557 | P09914 |
| interferon-inducible protein 9-27 | J04164 | P13164 |
| interleukin-1 beta convertase precursor (IL-1BC); IL-1 beta converting enzyme (ICE); p45; caspase-1 | U13699; M87507; | P29466 |
| (CASP1) | X65019 | |
| interleukin-1 receptor type II precursor (IL-1R2); IL-1R-beta | X59770 | P27930 |
| interleukin-16 (IL-16); lymphocyte chemoattractant factor (LCF) | M90391 | Q14005 |
| interleukin-2 receptor gamma subunit (IL-2R gamma; IL2RG); cytokine receptor common gamma chain | D11086 | P31785 |
| precursor; p64 | | |
| interleukin-6 receptor alpha subunit precursor (IL-6R-alpha; IL6R); CD126 antigen | M20566; X12830 | P08887 |
| I-rel (RELB) | M83221 | Q01201 |
| leukocyte IgG receptor (FC-gamma-R) | J04162 | P08637 |
| lipoprotein-associated coagulation inhibitor | J03225 | P10646 |
| low affinity immunoglobulin gamma FC receptor II-A precursor (FC-gamma RII-A; | M31932 | P12318 |
| FCRII-A; IgG FC receptor II-A); CD32 antigen | | |
| low-density lipoprotein receptor-related protein LR11 precursor | Y08110 | Q92673 |
| L-selectin precursor; lymph node homing receptor (LNHR); leukocyte adhesion molecule 1 | M25280 | P14151 |
| (LAM1) leukocyte surface leu-8 antigen; GP90-MEL; leukocyte-endothelial cell | | |
| adhesion molecule 1 (LECAM1); CD62L antigen; SELL | | |
| LUCA2; lysosomal hyaluronidase 2 (HYAL2); PH-20 homolog | U09577 | Q12891 |
| lymphocyte antigen | M81141 | Q30099 |
| lymphoid-restricted homolog of SP100 protein (LYSP100) | U36500 | Q13342 |
| lymphotoxin-beta (LT-beta; LTB); tumor necrosis factor C (TNFC) | L11015 | Q06643 |
| lysosomal acid lipase/cholesteryl ester hydrolase precursor (LAL); acid cholesteryl ester hydrolase; sterol | M74775 | P38571 |
| esterase; lipase A (LIPA); cholesteryl esterase | | |
| lysosomal pro-X carboxypeptidase | L13977 | P42785 |
| macrophage colony stimulating factor I receptor precursor (CSF-1-R); fms proto-oncogene (c-fms); CD115 | X03663 | P07333 |
| macrosialin precursor | S57235 | P34810 |
| manic fringe | U94352 | O00587 |
| matrix metalloproteinase 17 (MMP17); membrane-type matrix metalloproteinase 4 (MT-MMP4) | X89576 | Q14850 |
| matrix metalloproteinase 9 (MMP9); gelatinase B; 92-kDa type IV collagenase precursor (CLG4B) | J05070; D10051 | P14780 |
| MHC class II HLA-DR-beta (DR2-DQW1/DR4 DQW3) precursor | M20430 | Q30166 |
| microsomal aminopeptidase N; myeloid plasma membrane glycoprotein CD13 | M22324 | P15144 |
| microtubule-associated protein 1B | L06237 | P46821 |
| migration inhibitory factor-related protein 14 (MRP14); calgranulin B; leukocyte L1 complex heavy subunit; | X06233 | P06702 |
| S100 calcium-binding protein A9 | | |
| migration inhibitory factor-related protein 8 (MRP8); calgranulin A; leukocyte L1 complex light subunit; | X06234 | P05109 |
| S100 calcium-binding protein A8; cystic fibrosis antigen (CFAG) | | |
| myeloid cell nuclear differentiation antigen (MNDA) | M81750 | P41218 |
| myotonin-protein kinase; myotonic distrophy protein kinase (MDPK); DM-kinase (DMK) | L19268 | Q09013 |
| neurogenic locus notch protein (N) | M99437 | Q04721 |
| neurogranin (NRGN); RC3 | Y09689 | Q92686 |
| neurotrophic tyrosine kinase receptor-related 3; TKT precursor | X74764 | Q16832 |
| neutrophil cytosol factor 2; neutrophil NADPH oxidase factor 1 (NCF1); p47-PHOX); 47-kDa autosomal | M25665 | P14598 |
| chronic granulomatous disease protein | | |
| neutrophil gelatinase-associated lipocalin precursor (NGAL); 25-kDa alpha-2- | X99133 | P80188 |
| microglobulin-related subunit of MMP9); lipocalin 2; oncogene 24P3 | | |
| ninjurin-1 | U72661 | Q92982 |
| NKG5 protein precursor; lymphokine LAG2; T-cell activation protein 519 | X54101 | P22749 |
| NT-3 growth factor receptor precursor (NTRK3); C-trk tyrosine kinase (TRKC) | U05012 | Q16288; |
| | | Q16289; Q12827 |
| nuclear receptor-related 1 | X75918 | P43354 |
| NuMA | Z11583 | Q14981 |
| osteoclast stimulating factor | U63717 | Q92882 |
| P126 (ST5) | U15131 | P78524 |
| P2X purinoceptor 1; ATP receptor P2X1 | X83688 | P51575 |
| P2X purinoceptor 5 (P2X5) | AF016709 | Q93086 |
| paxillin | U14588 | P49023 |
| PC8 precursor | U33849 | Q16549 |
| peripheral myelin protein 22 (PMP22); CD25 protein; SR13 myelin protein | D11428 | Q01453 |
| peroxisomal bifunctonal enzyme | L07077 | Q08426 |
| phenol-sulfating phenol sulfotransferase 1 (PPST1); thermostable phenol sulfotransferase (TS-PST); | U09031 + | P50225 + P50226 + |
| HAST1/HAST2; ST1A3; STP1 + PPST2; ST1A2; STP2 + monoamine-sulfating phenol sulfotransferase | U28170 + L19956 | P50224 |
| phospholipase C beta 2 (PLC-beta 2; PLCB2); 1-phosphatidylinositol 4,5-bisphosphate phosphodiesterase | M95678 | Q00722 |
| beta 2 | | |
| phosphoribosyl pyrophosphate synthetase subunit I | D00860 | P09329 |
| PIG7 | AF010312 | Q99732 |
| pim-1 proto-oncogene | M54915 | P11309 |
| platelet basic protein precursor (PBP); connective tissue activating peptide III (CTAP III); low-affinity | M54995; M38441 | P02775 |
| platelet factor IV (LA PF4); beta thromboglobulin (beta TG); neutrophil activating peptide 2 (NAP2) | | |
| platelet endothelial cell adhesion molecule | HS78146 | P16284 |
| platelet membrane glycoprotein IIB precursor (GP2B); integrin alpha 2B (ITGA2B); CD41 antigen | M34480; J02764 | P08514 |
| platelet membrane glycoprotein IIIA precursor (GP3A); integrin beta 3 (ITGB3); CD61 antigen | J02703; M25108 | P05106; |
| | | Q13413; Q16499 |
| platelet-activating factor receptor (PAFR) | D10202 | P25105 |
| platelet-derived growth factor A subunit precursor (PDGFA; PDGF-1) | X06374 | P04085 |
| PRB-binding protein E2F1; retinoblastoma-binding protein 3 (RBBP3); retinoblastoma-associated protein 1 | M96577 | Q01094; |
| (RBAP1); PBR3 | | Q92768; |
| | | Q13143 |
| prostaglandin G/H synthase 1 | | P23219 |
| protein-tyrosine phosphatase 1C (PTP1C); hematopoietic cell protein-tyrosine phosphatase; SH-PTP1 | X62055 | P29350 |
| prothrombin precursor; coagulation factor II | V00595 | P00734 |
| proto-oncogene tyrosine-protein kinase lck; p56-lck; lymphocyte-specific protein tyrosine | U07236 | P06239 |
| kinase (LSK); T-cell-specific protein-tyrosine kinase | | |
| P-selectin precursor (SELP); granule membrane protein 140 (GMP140); PADGEM; CD62P antigen; | M25322 | P16109 |
| leukocyte-endothelial cell adhesion molecule 3 (LECAM3) | | |
| purine-rich single-stranded DNA-binding protein alpha (PURA) | M96684 | Q00577 |
| rab geranylgeranyl transferase alpha subunit | Y08200 | Q92696 |
| rab geranylgeranyl transferase beta subunit | Y08201 | P53611; Q92697 |
| RalB GTP-binding protein | M35416 | P11234 |
| ras-related C3 botulinum toxin substrate 2; p21-rac2; small G protein | M64595; M29871 | P15153 |
| ras-related protein RAB5A | M28215 | P20339 |
| related to receptor tyrosine kinase (RYK) | S59184 | P34925 |
| replication protein A 70-kDa subunit (RPA70; REPA1; RF-A); single-stranded DNA-binding protein | M63488 | P27694 |
| rho GDP dissociation inihibitor 2 (RHO GDI2; RHO-GDI beta); LY-GDI; ARHGDIB; GDID4 | L20688 | P52566 |
| rho-GAP hematopoietic protein C1 (RGC1); KIAA0131 | X78817 | P98171 |
| rho-related GTP-binding protein (RHOG); ARHG | X61587 | P35238 |
| ribonuclease 6 precursor | U85625 | O00584 |
| ribosomal protein S6 kinase II alpha 1 (S6KII-alpha 1); ribosomal S6 kinase 1 (RSK1) | L07597 | Q15418 |
| S100 calcium-binding protein A1; S-100 protein alpha chain | X58079 | P23297 |
| SCGF-beta | D86586 | BAA21499 |
| SEC7 homolog B2-1 | M85169 | Q15438 |
| selectin P ligand | U02297 | Q14242; Q12775 |
| semaphorin; CD100 | U60800 | Q92854 |
| serum response factor (SRF) | J03161 | P11831 |
| SH3-binding protein 2 | AF000936 | P78314 |
| signaling inositol polyphosphate 5 phosphatase; SIP-110 | U50040 | Q13544 |
| sonic hedgehog (SHH) | L38518 | Q15465 |
| specific 116-kDa vacuolar proton pump subunit | U45285 | Q13488 |
| steroid 5-alpha reductase 1 (SRD5A1); 3-oxo-5-alpha steroid 4 dehydrogenase 1 | M32313; M68886 | P18405 |
| stromal cell derived factor 1 receptor (SDF1 receptor); fusin; CXCR4; leukocyte-derived seven | D10924 | P30991 |
| transmembrane domain receptor (LESTR); LCR1 | | |
| superoxide dismutase 2 | M36693 | P04179 |
| T-cell surface glycoprotein CD3 epsilon subunit precursor; T-cell surface antigen T3/leu-4 epsilon subunit | X03884 | P07766 |
| (T3E) | | |
| tenascin precursor (TN); hexabrachion (HXB); cytotactin; neuronectin; GMEM; miotendinous antigen; | X78565; M55618 | P24821; |
| glioma-associated extracellular matrix antigen | | Q15567; Q14583 |
| thrombospondin 1 precursor (THBS1; TSP1) | X14787 | P07996 |
| thymidine phosphorylase precursor (TDRPase); platelet- derived endothelial cell growth factor (PDECGF); | M63193 | P19971; Q13390 |
| gliostatin | | |
| TNF-related apoptosis inducing ligand (TRAIL); APO-2 ligand (APO2L) | U57059 | P50591 |
| TRAIL receptor 3; decoy receptor 1 (DCR1) | AF016267 | O14755 |
| transcription factor Spi-B | X66079 | Q01892 |
| transcriptional regulator interferon-stimulated gene factor 3 gamma subunit (ISGF3G); interferon-alpha (IFN- | M87503 | Q00978 |
| alpha) responsive transcription factor subunit | | |
| transforming growth factor-beta 3 (TGF-beta3) | J03241 | P10600 |
| tuberin; tuberous sclerosis 2 protein (TSC2) | X75621 | P49815 |
| type I cytoskeletal 18 keratin; cytokeratin 18 (K18) | M26326 | P05783 |
| type II cytoskeletal 6 keratin: cytokeratin 6A (CK6A); K6A keratin (KRT6A) + CK6B; | J00269 + L42592 + | P02538 |
| KRT6B + CK6C; KRT6C + CK6D; KRT6D + CK6E; KRT6E + CK6F; KRT6F | L42601 + L42610 + | |
| | L42611 + L42612 | |
| tyrosine-protein kinase lyn | M16038 | P07948 |
| tyrosine-protein kinase receptor UFO precursor; axl oncogene | M76125 | P30530 |
| vascular endothelial growth factor B precursor (VEGFB) + VEGF-related factor isoform VRF 186 | U48801; U43369 | P49765 |
| vav oncogene | X16316 | P15498 |
| v-erbA related protein (EAR2) | X12794 | P10588 |
| versican core protein precursor; large fibroblast proteoglycan; chondroitin | U16306; X15998; | P13611 |
| sulfate proteoglycan core protein 2; glial hyaluronate-binding protein (GHAP) | U26555; D32039 | |
| vitamin K-dependent protein S | Y00692 | P07225 |

For example, it was found that 17 of the genes differentially expressed in human neointima encode transcriptional regulators. mRNA levels for 14 transcription factors were induced in neointima and 3 showed a decreased expression (FIG. 15). Some transcription factors of the former group have previously been related to proliferation and apoptosis of SMCs, such as HMG-1, E2F1, IRF-1, Fli-1, and with pro-inflammatory signaling in human neointima, such as IRF-1, IRF-7 and RelB. The following transcription factors were upregulated: E2F1, estrogen-related receptor alpha, ets domain protein elk-3, fli-1 oncogene, HMG-1, interferon regulatory factor 1, interferon regulatory factor 7, ISGF3-gamma, nuclear receptor-related 1, RELB, transcription factor Spi-B, vav oncogene, v-erbA related protein, vitamin D3 receptor; whereas the following were downregulated: homeobox protein HOXB7, early growth response protein 1, serum response factor.

Striking changes seem to take place in the expression of transcription factors of the Ets family. Whereas Spi-B, the fli-oncogene, and the Ets-repressor Elk-3 were induced in neointima, the Ets transcription factor Egr-1 was repressed (FIGS. 14 and 15).

Furthermore, a number of genes involved in controlling or mediating proliferative responses were differentially expressed between neointima and control groups. The platelet-derived growth factor (PDGF)-A and angiotensinogen genes, whose products act on SMCs as mitogens, were exclusively expressed in neointima (FIG. 14). Angiotensin is known to be upregulated by insulin and to induce the expression of PDGF-A in SMCs. As a sign of ongoing proliferation, several genes known to be expressed with the G1/S transition of the cell cycle were found to be upregulated in neointima. Those include transcription factor E2F1, 70-kDa replication protein A, oncogene product Pim-1 and geranylgeranyl transferase. In addition, upregulation of the cell-cycle regulated histone H4, which is expressed in the G/S1 and S-phase of the cell cycle indicating ongoing proliferation in human neointima, was observed.

Reprogramming of cell growth in neointima evidently led to induction of several genes in neointima encoding proteins with functions in different signal transduction pathways, including the cell surface receptors EDG-1, EDG-4, insulin receptor and P2X purinoceptor 5, and other signaling proteins like the ribosomal protein S6 kinase II alpha 1, farnesyltransferase, phospholipase C beta 2, growth factor receptor-bound protein 2, and the small G proteins CDC42, RhoG, p21-Rac2 and RalB. The enzyme farnesyltransferase catalyzes the essential post-translational lipidation of Ras and several other signal transducing G proteins. G proteins, like p21-Rac2, CDC42 and RhoG play pivotal roles in signal transduction pathways leading to cell migration and cell proliferation. Likewise, agonist-stimulated 1,4,5-triphosphate (IP3) production by phospholipase C beta 2 in smooth muscle requires G protein activation and activated Rac and Cdc42 associate with PI 3 kinase that plays an important role in the activation of the p70 S6 kinase. The p70 S6 kinase (p70S6K) is an important regulator of cell cycle progression to enter G1 phase and to proceed to S phase in response to growth factors and mitogens. It is involved in multiple growth factor related signal transduction pathways that are known to play pivotal roles in neointima formation, like angiotensin, endothelin and PDGF. In line with upregulation of p70 S6 kinase, significant upregulation of the FK506-binding protein (FKBP) 12 at mRNA (FIG. 14) and protein level in neointima was found.

It was observed that a number of genes encoding inhibitors of cell cycle progression were expressed in quiescent media but significantly downregulated in neointima (FIG. 14). Those included CIP1, p16-INK4, metallothionein, TGF-beta3, mammary-derived growth inhibitor, FrzB and the Gadd45 beta and gamma subunits.

Additionally, upregulation of genes in human neointima encoding proteins with pro-apoptotic function, like caspase-1, DAP-1 and APO-2 ligand, as well as upregulation of genes encoding proteins with anti-apoptotic function, like BAG-1, BCL-2-related protein A1 and the Trail receptor 3 (FIG. 14) was found.

Finally, the human neointima transcriptome showed upregulation of 32 genes related to IFN-γ signaling (FIG. 16). The IFN-γ receptor alpha was expressed in neointima, proliferating CASMCs and—to a lesser degree—in blood cells; whereas the IFN-γ receptor beta was mainly expressed in neointima specimen. Likewise, an upregulation of Pyk2 was observed.

Upregulation of the IFN-γ regulated genes for caspase-1, caspase-8 and DAP-1 was found in human neointima. However, mRNAs for the anti-apoptotic proteins BAG-1, Pim-1 (both regulated by IFN-γ) and BCL-2-related protein A1 were also upregulated in neointima versus control (FIG. 14).

Numerous genes with functions in inflammatory responses were found activated in human neointima. Pro-inflammatory gene patterns came from infiltrating inflammatory cells such as macrophages and T lymphocytes (e.g., CD11b, CD3) (FIG. 14C) or from neointimal SMCs (e.g., prostaglandin G/H synthase 1, phospholipase A2, heat shock protein 70, C5a anaphylatoxin receptor, IFN-γ receptor) (FIGS. 14A and B).

The selective expression of CD40 in neointima deserves attention (FIG. 14A). CD40 is a member of the TNF receptor family that was initially described on the surface of B cells.

The following cytoskeletal, extracellular matrix and cell adhesion changes in neointima were observed:

An upregulation of connexin43 and of cytokeratin-18 in neointima as is seen in proliferating CASMC (FIG. 14B, upper panel), whereas the expression of desmin was strongly reduced in neointima (FIG. 14D, upper panel).

Whereas the transcription of different collagen subtypes and tenascin were reduced in neointima (FIG. 14D, upper panel), expression of thrombospondin-1 and versican were upregulated (FIG. 14B, upper panel).

A number of genes encoding adhesion molecules, including P-selectin, ICAM2 and cadherin16, were found highly expressed in neointima but not in SMCs, blood cells or control vessels (FIG. 14A, upper panel). A number of other adhesion molecules were similarly expressed in neointima, cultured SMCs (FIG. 14B) and blood cells (FIG. 14C). Neointima appears to downregulate expression of certain adhesion molecules that are normally expressed in media/intima of arteries, such as integrins α7B, α3 or MUC18.

### EXAMPLE VII

### Upregulated Genes of the IFN-γ Signaling Pathway

As shown herein above, the expression of 2,435 genes of known function in atherectomy specimen of 10 patients with in-stent restenosis, blood cells of 10 patients, normal coronary artery specimen of 11 donors, and cultured human coronary artery smooth muscle cells was investigated and 224 genes that were differentially expressed with high statistical significance (p<0.03) between neointima and control tissue were identified. In particular, 32 upregulated genes that are related to interferon-γ signaling were identified in neointima.

The IFN-γ receptor alpha was expressed in neointima, proliferating CASMCs and—to a lesser degree—in blood cells; whereas the IFN-γ receptor beta was mainly expressed in neointima specimen.

IFN-γ signals via a high-affinity receptor containing an α- and β-receptor chain. Interstingly, TH1 cells use receptor modification to achieve an IFN-γ-resistant state (Pernis, Science 269 (1995), 245-247). The subtype-specific difference in the activation of the IFN-γ signaling pathway of type 1 and type 2 T helper cells is due to a lack of IFN-γ receptor β in type 1 T cells. Therefore, the here presented data would argue that a high affinity IFN-γ receptor containing both chains is mainly expressed in smooth muscle cells of the neointima.

Consistent with an activation of IFN-γ signaling, upregulation of two transcription factors in neointima that are essential for IFN signalling were found: IRF-1 and ISGF3γ (p48). These transcription factors are known to be transcriptionally upregulated by IFN-γ (Der, Proc. Natl. Acad. Sci. 95 (1998), 15623-15628), and both are key players in IFN-γ signalling (Matsumoto, Biol. Chem. 380 (1999), 699-703; Kimuar, Genes Cells 1 (1996), 115-124; Kirchhoff, Nucleic Acids Res. 21 (1993), 2881-2889; Kano, Biochem. Biophys. Res. Commun. 257 (1999), 672-677). Likewise, upregulation of the tyrosine kinase Pyk2 was observed, which has been shown to play a role in the signal transduction by angiotensin in SMCs (Sabri, Circ. Res. 83 (1998), 841-851). Pyk2 is selectively activated by IFN-γ and inhibition of Pyk2 in NIH 3T3 cells results in a strong inhibition of the IFN-γ-induced activation of MAPK and STAT1 (Takaoka, EMBO J. 18 (1999), 2480-2488.

A key event in IFN-γ-induced growth inhibition and apoptosis is the induction of caspases (Dai, Blood 93 (1999), 3309-3316). It has been shown that IRF-1 induces expression of caspase-1 leading to apoptosis in vascular SMCs (Horiuchi, Hypertension 33 (1999), 162-166), and that apoptotic SMCs and macrophages colocalize with caspase-1 in atherosclerosis (Geng, Am. J. Pathol. 147 (1995), 251-266). In this studies, upregulation of the IFN-γ-regulated genes for caspase-1, caspase-8 and DAP-1 in human neointima was found. However, mRNAs for the the anti-apoptotic proteins BAG-1, Pim-1 (both regulated by IFN-γ) and BCL-2-related protein A1 were also upregulated in neointima versus control (FIG. 16), supporting the notion that proliferation and apoptosis occur simultaneously in human neointima with a preponderance of proliferation.

Coordinated regulation of genes whose products act at different steps in the neointima process was a recurring theme of our gene expression analysis. Regarding the IFN-γ pathway, not only the genes for the complete receptor, the main transcription factors, components of the signal transduction pathway (Dap-1, BAG-1, Pim-1, IFN-γ-inducible protein, IFN-inducible protein 9-27) were induced but also several target genes of the IFN-γ pathway, like CD40, CD13 and thrombospondin-1 (FIG. 16).

The IFN-γ-regulated gene cluster was expressed in the neointima specimen but some of the relevant genes, like IRF-1, were also expressed in blood samples. To identify the cell type that predominantly contributed to the IFN-γ regulated pattern, frozen sections of neointima specimen from coronary in-stent restenosis (n=3) and from restenosis of peripheral arteries (n=6) were stained with antibodies specific for IRF-1. This protein was chosen because it is an essential component of the IFN-γ signal transduction pathway (Kimura, loc. cit.) and was expressed coordinately with the other genes in the cluster (FIG. 16). Immunohistochemical analysis showed strong nuclear and cytoplasmic staining of IRF-1 in neointimal SMCs of a carotid restenosis (FIG. 17) and of coronary in-stent restenosis (FIG. 18), as identified by their spindle-shaped nuclei and by staining with the smooth muscle cell marker alpha-actin (FIG. 18). The nuclear staining of IRF-1 in in-stent restenosis (FIG. 18) indicated that the IRF-1 transcription factor is also activated. SMCs in control media of carotid arteries did not show IRF-1 staining (FIG. 17). These data were confirmed on paraffin serial sections. Detection of IRF-1 was performed immunohistochemically on paraffin serial sections of four neointima specimen from coronary in-stent restenosis, 3 neointima specimen from femoral arteries and 3 specimen from carotid arteries. Staining of coronary in-stent restenosis specimen with IRF-1 specific antibodies demonstrated a nuclear staining pattern of most smooth muscle cells in these specimen. Neointima is mainly composed of α-actin positive smooth muscle cells, while only few CD3-positive T-cells were detected.

Immunohistochemical detection of IRF-1 in a carotid restenosis specimen revealed a perinuclear cytoplasmic staining in neointimal smooth muscle cells while no immunostaining was observed in healthy control media. As already mentioned before, CD3-positive cells were much less abundant in the specimen (FIG. 18) than SMCs (FIG. 18), indicating that SMCs contributed mostly to the increased IRF-1 expression in human neointima.

The presence of IFN-γ in human atherosclerotic lesions is well established (Ross, N. Engl. J. Med. 340 (1999), 115-126) although its role remains unclear. Whereas IFN-γ inhibits proliferation and induces apoptosis in SMCs in vitro (Horiuchi, loc. cit.; Warner, J. Clin. Invest 83 (1989), 1174-1182), absence of IFN-γ reduces intima hyperplasia in mouse models of atheroma and transplant arteriosclerosis (Gupta, J. Clin. Invest 99 (1997), 2752-2761; Raisanen-Sokolowski, Am. J. Pathol. 152 (1998), 359-365). In line with this observation, it was shown that IFN-γ induces arteriosclerosis in absence of leukocytes in pig and human artery tissues by their insertion into the aorta of immunodeficient mice (Tellides, Nature 403 (2000), 207-211).

The role of infiltrating T lymphocytes in neointima of in-stent restenosis has not been examined yet. In this study it was shown that CD3-positive cells can be detected by immunobiochemists in 3 out of 4 neointima samples (see FIG. 18), and a CD3-specific hybridization signal on cDNA arrays with 7 out of 10 neointima specimen was obtained (FIG. 18). IFN-γ-related expression patterns were also observed in samples negative for CD3 as examined by either method, suggesting that the cytokine could act on neointima in a paracrine fashion over some distance with no need for massive T cell infiltration.

While T cells and the pro-inflammatory cytokine IFN-γ are known to play an important role in atherosclerosis (Ross, loc. cit.), their role in the development of neointima is largely unexplored. The here provided data suggest an important role of IFN-γ in the pathophysiology of neointimal hyperplasia.

### EXAMPLE VIII

### Preparation of a Surrogate Cell Line

A surrogate cell line for a pathologically modified cell and/or tissue may be prepared by the following steps:

a) Definition of the Transcriptome/Gene Expression Pattern of the Diseased Tissue:

Microscopic specimen of diseased tissue may be obtained by either atherectomy, debulking, biopsy, laser dissection of diseased tissue or macroscopic surgical dissection of diseased tissue. After acquisition, microscopic specimen are immediately frozen in liquid nitrogen and kept in liquid nitrogen until mRNA preparation is performed in order to preserve the in vivo status of the samples' transcriptomes.

The cells in such samples express a particular set of genes which is reflected by the presence of distinct mRNA molecules occuring at various concentrations. The entirety of mRNA molecules and their relative amounts in a given clinical sample is referred to as the transcriptome. The transcriptome of a diseased tissue is expected to be different from that of a healthy tissue. The differences relate to the up- or downregulated expression of genes involved in causing, maintaining or indicating the diseased state of the tissue. The analysis of the transcriptome is typically limited by the number of cDNA elements a particular array carries.

mRNA preparation and amplification is carried out according to the method of the invention and described herein above.

In particular, microscopic specimen of diseased tissue are quick-frozen and kept in liquid nitrogen until mRNA preparation and cDNA synthesis is performed as described herein above. Frozen tissue is ground in liquid nitrogen and the frozen powder dissolved in Lysis buffer according to the procedure of RNA preparation. The lysate is centrifuged for 5 min at 10,000 g at 4° to remove cell debris. RNA can be prepared as total RNA or as mRNA as described ein (Schena, Science 270 (1995), 467-470), in Current Protocols, in the Clontech manual for the Atlas cDNA Expression Arrays or as described in (Spirin, Invest. Ophtalmol. Vis. Sci. 40 (1999), 3108-3115), as described in (Chee, Science 274 (1996), 610-614; Alon, Proc. Natl. Acad. Sci. 96 (1999), 6745-6750; Fidanza, Nucleosides Nucleotides 18 (1999), 1293-1295; Mahadevappa, Nat. Biotechnol. 17 (1999), 1134-1136; Lipshutz, Nat. Genet. 21 (1999), 20-24) for the Affymetrix arrays or as described by Qiagen.

cDNA preparation and labeling can be performed as described by Clontech or Affymetrix in the user's manual for the arrays hybridization kits or as described in (Spirin, loc. cit.; Chee, loc. cit.; Alon, loc. cit.; Fidanza, loc. cit.; Mahadevappa, loc. cit.; Lipshutz, loc. cit.). Additionally, amplified cDNA can be used. Preparation of cDNA amplificates and labeling of amplificated cDNA can be performed as described herein above or by Spirin (loc. cit.).

Obtained, labeled cDNA can be employed in hybridization assays. Hybridization of labeled cDNA and data analysis can be performed under conditions as described in the user's manual from Clontech's Atlas™ cDNA Expression Arrays User Manual or in the manufacter's manual of Affymetrix or as described by (Spirin, loc. cit.; Chee, loc. cit.; Alon, loc. cit.; Fidanza, loc. cit.; Mahadevappa, loc. cit.; Lipshutz, loc. cit.).

b) Definition of the Transcriptome/Gene Expression Pattern of Control Tissue

To identify disease-specific gene expression patterns, the gene expression pattern of the diseased tissue can be compared to control material from healthy donors. In the case of atherectomy material this can be healthy media and intima of non-elastic, i.e., muscular arteries. In the case of heart muscle biopsies or kidney biopsies, healthy control tissue can be used that is collected in the course of the operation. Additionally, gene expression pattern of cells of neighbouring unaffected tissue or of infiltrating cells, like blood, cells can be analyzed. Based upon the celluar characterization of a tissue by immunohistochemical analysis using antibodies to cell marker proteins, transcriptome can be determined from cultured human cell lines of the same type. (Example: arteries stain positive for smooth muscle cells and endothelial cells; consequently transcriptomes are obtained from cultured human smooth muscle and endothelial cells).

mRNA preparation and amplification can be carried out as described herein above and in accordance with the method of the present invention. Obtained (labeled) cDNA may be employed in hybridization assays as described herein above.

c) Determination of a Relevant Set of Disease Specific Genes

To determine disease-specific gene expression patterns first the gene expression pattern of the diseased tissue should be compared to the gene expression pattern of healthy control tissue. For comparison, the mean expression value of at a sufficient number of diseased specimen (e.g., 10) and the same number of control specimen should be compared. Genes with an expression ratio >2.5-fold between the the two groups should be analyzed for their relative expression in one group: there should be >5/10 positive in one group, if there are 0/10 in the other or at least 7/10 in one group if there are maximally 3/10 positive in the other group. Additionally, these data should be analysed statistically to define genes with an p<0.05 with e.g. the Wilcoxon test as described in the manual of SPSS 8.0.

Genes selected based upon their significant over- or underexpression by a factor of 2.5 are refered to as aberrantly regulated in the diseased tissue, or as diseases-related genes. Disease-related genes genes are then grouped by the functions of encoded proteins. e.g. genes encoding proteins of the signalling pathway, cytokines, chemokines, hormones, their receptors, proteins specific or infiltrating cells, or proteins involved in extracellular matrix, cell adhesion, migration, cell division, cell cycle arrest. Likewise genes of unknown function, as available thorugh public EST data bases, can be identified as being disease-related.

d) Screen for a Cell Line With a Transcriptome Most Closely Resembling That of Diseased Tissue

Drugs that can potentially regulate the expression of diseased genes can be discovered by screening large libraries of chemicals or biologics. In order to identify such drugs, a screening cell line must be available that faithfully reflects the transcriptome of the diseased tissue and is avaiabale in large quantaties for the performance of a comprehensive drug screen. Moreover information is needed of how the drug candidate should alter the transcriptome of the cell line that has characteristics of the transcriptome of the diseased tissue. This information is obtained from the transcriptome of the healthy control tissue. The drug should be able to re-estsblish features of a “healthy” transcriptome.

A human cell line, which is most similiar to the cellular origin of the diseased tissue, e.g, coronary artery smooth muscle cells for atherectomy, HepG2 cells for liver diseases, renal cells for kidney diseases or cardiomyoblasts for heart muscle disease should be used. Cells should be grown under standard conditions as described in the manufacter's manual like the ones from ATCC.

Transcriptome analysis/gene expression pattern analysis can be performed as described for the diseased and the control tissue and gene expression pattern should be compared to the gene expression pattern of the diseased and the healthy tissue. For generating a surrogate screening cell line, the cell line which shows a transcriptome most similar to the diseased transcriptome should be selected.

e) Adaptation of a Cell Line to Mimick Diseased Transcriptome/gene Expression Pattern

In order to generate a surrogate screening cell line for the diseased tissue, it may be necessary to adapt the transcriptome of the selected cell line to the transcriptome of the diseased tissue. This can on the one hand be achieved by incubation of the cell line with compounds such as cytokines or hormones, that had been shown to play an important role in the gene expression pattern of the diseased tissue. Likewise such compounds can be identified by transcriptome analysis of diseased tissue as exemplified with neoinitima where evidence for a role of interferon-gamma was obtained. Instead of addition of compounds with relevance for the disease, the screening cell line can be conditioned by co-culture with other cell types relevant for the pathophysiology of the disease. Such cells can for instance be inflammatory cells, like macrophages or T cells, that migrate into the diseased tissue and by released factors or cell-cellcontact contribute to the disease-specific gene expression pattern. In each case, transcriptome analysis of the surrogate line must identify the optimal addition to generate a disease-specific expression pattern.

Compounds that can be used for adapting the transcriptome of a surogate cell line to the diseased state comprise cytokines, growth factors, small molecule compounds (drugs), or peptides and peptidomimetics. Cell lines that can be used for such an adaptation comprise human monocytic cell lines, like U937, THP-1 or Monomac-6, or human T-cell lines like Jurkat. The co-culture/treatment conditions leading in the surrogate cell line to a state closest to the diseased transcriptome are selected for drug screening.

In the following, a specific example should illustrate the preparation of a surrogate. In particular, a surrogate cell (line) for restenotic tissue is prepared by the following steps:

a) Aquisition of In-stent Restenotic Tissue

Patients

The in-stent restenosis study group consisted of 13 patients who underwent separate atherectomy procedures by X-sizer within the renarrowed stent between 4-23 month after primary stent implantation. All patients gave informed consent to the procedure and received 15,000 units heparin before the intervention followed by intravenous heparin infusion, 1,000 units/h for the first 12 h after sheat removal as standard therapy. All patients received aspirin, 500 mg intravenously, before catherisation, and postinterventional antithrombotic therapy consisted of ticlopidine (250 mg bds) and aspirin (100 mg bds) throughout the study.

Sample Preparation

Atherectomy specimen were immediately frozen in liquid nitrogen after debulking of the lesion, and kept in liquid nitrogen until mRNA preparation was performed as described. For histology and immunhistochemistry of the in-stent restenotic tissue from coronary arteries (n=3), the samples were fixed in 4% paraformaldehyd and embedded in paraffin as described.

Morphological Characterization of Restenotic Tissue

Immunohistochemistry for cell typing was performed on paraffin-embedded sections of three neointima specimen from coronary in-stent restenosis and, for detection of FKBP12, on frozen sections of four neointima specimen from carotid restenosis. Three μm serial sections were mounted onto DAKO ChemMate™ Capillary Gap Microscope slides (100 μm) baked at 65° C. overnight, deparaffinized and dehydrated according to standard protocols. For antigen retrieval, specimens were boiled 4 min in a pressure cooker in citrate buffer (10 mM, pH 6.0). Endogenous peroxidase was blocked by 1% H2O2/methanol for 15 minutes. Unspecific binding of the primary antibody was reduced by preincubation of the slides with 4% dried skim milk in Antibody Diluent (DAKO, Denmark). Immunostaining. was performed by the streptavidin-peroxidase technique using the ChemMate Detection Kit HRP/Red Rabbit/Mouse (DAKO, Denmark) according to the manufacturer's description. The procedures were carried out in a DAKO TechMate™ 500 Plus automated staining system. Primary antibodies against smooth muscle actin (M0635, DAKO, Denmark; 1:300), CD3 (A0452, DAKO, Denmark; 1:80), MAC387 (E026, Camon, Germany; 1:20) and FKBP12 (SA-218, Biomol, Germany, 1:20) were diluted in Antibody Diluent and incubated for 1 h at room temperature. After nuclear counterstaining with hematoxylin, the slides were dehydrated and coverslipped with Pertex (Medite, Germany).

The Cellular Composition of Debulked In-stent Restenotic Material

Representative samples obtained from x-sizer treatment of a neointimal hyperplasia were analyzed by immunhistochemistry in order to determine its cellular composition. FIG. 7A shows an E.-van-Giesson staining of a section cut from a small sample of debulked restenotic material. With this staining procedure, collagen fibers stain red, fibrin stains yellow and cytoplasm of smooth muscle cells stains dark-yellow-brown. The majority of the volume of debulked material was composed of loose extracellular matrix-like collagen fibers stained in light red. Yellow fibrin staining was barely detectable. Cells with spindle-shaped nuclei and a yellow/brown-stained cytoplasm were frequent. Their identity as smooth muscle cells and their high abundance in restenotic material was supported by immunostaining with an antibody against smooth muscle α-actin (FIG. 7B). There, the staining pattern of a section from an entire specimen as used for gene expression analysis is shown. As described below, such samples also gave raise to a strong smooth muscle-specific α-actin mRNA signal (see FIG. 8). These results support findings from previous studies (Kearney, Circulation 95 (1997), 1998-2002; Komatsu, Circulation 98 (1998), 224-233; Strauss, J. Am. Coll. Cardiol. 20 (1992), 1465-1473) demonstrating that the main cell type found in neointima is derived from smooth muscle cells. As described in the literature (Kearney, loc. cit.; Komatsu, loc. cit.; Strauss, loc. cit.) mononuclear infiltrates could also be identified in some areas of debulked restenotic tissue specimen. These infiltrates consisted mainly of macrophages and to a lesser degree of t-lymphocytes. No b-lymphocytes were detectable in the restenotic tissue by using an antibody against CD20 for immunhistochemical staining.

b) Transcriptome Analysis of Restenotic Material

Transcriptome analysis of neointima was performed using the method of mRNA amplification as described herein above.

mRNA Preparation

Microscopic specimen diseased tissue were quick-frozen and kept in liquid nitrogen until mRNA preparation and cDNA synthesis was performed. Frozen tissue is ground in liquid nitrogen and the frozen powder dissolved in Lysis/Binding buffer (100 mM Tris-HCl, pH 7.5, 500 mM LiCl, 10 mM EDTA, pH 8.0, 1% LiDS, 5 mM dithiothreitol (DTT)) and homogenized until complete lysis is obtained. The lysate is centrifuged for 5 min at 10,000 g at 4° to remove cell debris. mRNA is prepared using the Dynbeads® mRNA Direct Kit™ (Dynal, Germany) following the manufacture's recommendation. Briefly, lysate was added to 50 μL of pre-washed Dynabeads Oligo (dT)25 per sample and mRNA was annealed by rotating on a mixer for 30 min at 4° C. Supernatant was removed and Dynabeads Oligo (dT)25/mRNA complex was washed twice with washing buffer containing Igepal (50 mM Tris-HCl, pH 8.0, 75 mM KCl, 10 mM DTT, 025% Igepal), and once with washing buffer containing Tween-20 (50 mM Tris-HCl, pH 8.0, 75 mM KCl, 10 mM DTT, 0.5% Tween-20).

Preparation of Amplified cDNA

cDNA is amplified by PCR using the procedure of Klein et al. (C. Klein et al.). First-strand cDNA synthesis is performed as solid-phase cDNA synthesis. Random priming with hexanucleotide primers is used for reverse transcription reaction. mRNAs are each reversely transcribed in a 20 μL reaction volume containing 1×First Strand Buffer (Gibco), 0.01 M DTT (Gibco), 0.25% Igepal, 50 μM CFL5c-Primer [5′-(CCC)5 GTC TAG A (NNN)2-3′], 0.5 mM dNTPs each (MBI Fermentas) and 200 U Superscript II (Gibco), and incubate at 44° C. for 45 min. A subsequent tailing reaction is performed in a reaction volume of 10 μL containing 4 mM MgCl2, 0.1 mM DTT, 0.2 mM dGTP, 10 mM KH2PO4 and 10 U of terminal deoxynucleotide transferase (MBI Fermentas). The mixture is incubated for 24 min at 37° C. cDNA is amplified by PCR in a reaction volume of 50 μL containing 1×buffer 1 (Expand™ Long Template PCR Kit, Boehringer Mannheim), 3% deionized formamide, 120 μM CP2-Primer [5′-TCA GAA TTC ATG (CCC)5-3′], 350 μM dNTP and 4.5 U DNA-Polymerase-Mix (Expand™ Long Template PCR Kit, Roche Diagnostics, Mannhein). PCR reaction is performed for 20 cycles with the following cycle parameters: 94° C. for 15 sec, 65° C. for 0:30 min, 68° C. for 2 min; for another 20 cycles with: 94° C. for 15 sec, 65° C. for 30 sec, 68° C. for 2:30+0:10/cycle min; 68° C. 7 min; 4° C. forever.

Expression of Specific Genes in Microscopic Human Tissue Samples

In order to optimally preserve the in situ mRNA levels, restenotic and control specimen were immediately frozen after harvest in liquid nitrogen and carefully lyzed as described in Materials and Methods. After PCR amplification of the synthesized cDNA the amount of the amplified cDNA was measured by a dot blot assay and found to be between 200-300 ng/μl. The quality of every amplified cDNA sample was tested by gene-specific PCR using primers detecting cDNAs for β-actin, smooth muscle cell α-actin and the ubiquitous elongation factor EF-1α. FIG. 8 shows a representative result with material from patient B and control media from donor b. In both specimen, PCR signals of the correct size from housekeeping genes β-actin and EF-1α were detectable in equivalent amounts (compare lanes 1 and 2 with lanes 4 and 5). Additionally, α-actin signals as marker for smooth muscle cells was obatined from each specimen (lanes 3 and 6). These results show that mRNA prepraration, cDNA synthesis and PCR amplification of cDNA is feasible with microscopic human restenosis samples.

Dig-dUTP Labeling of cDNA Probes

25 ng of each cDNA is labeled with Digoxigenin-11-dUTP (Dig-dUTP) (Roche Diagnostics) during PCR. PCR is performed in a 50 μL reaction with 1×Puffer 1, 120 μM CP2 primer, 3% deionized formamide, 300 μM dTTP, 350 μM dATP, 350 μM dGTP, 350 μM dCTP, 50 μM Dig-dUTP, 4.5 U DNA-Polymerase-Mix. Cycle parameters are: one cycle: 94° C. for 2 min; 15 cycles: 94° C. for 15 sec, 63° C. for 15 sec, 68° C. for 2 min; 10 cycles: 94° C. for 15 sec, 68° C. for 3 min+5 sec/cycle; one cycle: 68° C., 7 min, 4° C. forever.

Hybridization of Clontech cDNA Arrays with dUTP-labeled cDNA Probes

cDNA arrays are prehybridized in DigEASYHyb solution (Roche Diagnostics) containing 50 mg/L DNAsel (Roche Diagnostics) digested genomic E. coli DNA, 50 mg/L pBluescript plasmid DNA and 15 mg/L herring sperm DNA (Life Technologies) for 12 h at 44° C. to reduce background by blocking non-specific nucleic acid-binding sites on the membrane. Hybridization solution is hybridized to commercially available cDNA arrays with selected genes relevant for cancer, cardiovascular and stress response (Clontech). Each cDNA template is denatured and added to the prehybridization solution at a concentration of 5 μg/ml Dig-dUTP-labeled cDNA. Hybridization was performed for 48 hours at 44° C.

Blots are subsequently rinsed once in 2×SSC/0.1% SDS and once in 1×SSC/0.1% SDS at 68° C. followed by washing for 15 min once in 0.5×SSC/0.1% SDS and twice for 30 min in 0.1×SSC/0.1% SDS at 68° C. For detection of Dig-labeled cDNA hybridized to the array, the Dig Luminescent Detection Kit (Boehringer, Mannheim) was used as described in the user manual. For detection of the chemiluminescence signal, arrays are exposed to chemiluminescence films for 30 min at room temperature. Quantification of array data was performed by scanning of the films and analysis with array vision software (Imaging Research Inc., St. Catharines, Canada). Background was subtracted and signals were normalized to the nine housekeeping genes present on each filter, whereby the average of the housekeeping gene expression signals was set to 1 and the background set to 0.

Each labeled probe was hybridized to three different commercial cDNA arrays which allowed for the expression analysis of a total of 2,435 known genes. FIG. 9 shows a representative hybridization pattern obtained with one array using probes prepared from restenotic tissue of patient B (panel A) and media of donor b (panel B). Consistent with the gene-specific analysis shown in FIG. 8, comparable hybridization signals were obtained with the positive control of human genomic cDNA spotted on the right and bottom lanes of the array and with cDNA spots of various housekeeping genes (see for instance, spots D). If a biological specimen was omitted from cDNA synthesis and PCR amplification reactions almost no hybridization signals were obtained (FIG. 9, panel C), showing that hybridization signals were almost exclusively derived from added samples and not from DNA contaminations in reagents or materials used.

Data Analysis

Quantification of array data was performed by scanning of the films and analysis with array vision software (Imaging Research Inc., St. Catharines, Canada). Background was subtracted and signals were normalized to the nine housekeeping genes present on each filter, whereby the average of the housekeeping gene expression signals was set to 1 and the background set to 0. For the logarithmic presentation shown in FIGS. 13A and 13B, values were multiplied by 1000. A mean value >0,05 in the average of all samples in one group was regarded as a positive signal. Differences in the mean expression level by a factor >2.5-fold between the study and the control group were further statistically analyzed.

c) Choice of Control Tissue

As the main cellular component of neointima consists of smooth muscle cells, media and media/intima were taken of healthy coronary arteries or as coronary arteries belong to the non-elastic but muscular arteries muscular arteries as control tissue.

The control group consisted of 6 specimen from coronary arteries from three different patients who underwent heart transplantation. Additionally, 5 specimen of muscular arteries of the gastrointestinal tract from five different patients were taken as control because coronary arteries belong histologically to muscular arteries. The control specimen were immediately frozen in liquid nitrogen. Prior to mRNA preparation, media and intima of the control arteries were prepared and examined for atherosclerotic changes by immunhistochemistry. If there were no atherosclerotic changes of the vessel morphology, the specimen (approx. 1×1 mm) were used as healthy control samples and mRNA and cDNA preparation and transcriptome analysis was performed as described above for neointimal tissue.

d) Definition of the Neointima-specific Gene Expression Profile

A total of 1,186 genes (48.7%) out of 2,435 yielded detectable hybridization signals on cDNA arrays with neointima and control samples over a 20-fold range of expression level (FIG. 13A) Thereof 352 genes (14.5%) appeared to be differentially expressed by a factor >2.5-fold between restenotic and control samples. However, expression levels considerably varied among individual samples (see, e.g., FIG. 9). A statistical analysis was therefore employed in order to identify those genes that are differentially expressed between study and control groups with high significance (see herein above). This way, 224 genes (9.6%) were identified that were differentially expressed by a factor of at least 2.5-fold between the restenosis study group and the control group with a significance in the Wilcoxon test of p<0.03. 167 (75%) genes thereof were found overexpressed and 56 genes (25%) underexpressed in the restenosis study group compared to the control group (FIG. 13B).

e) Choice of Surrogate Cell Line

Human neointima consists of a heterogenous cell population. It was therefore attempted to relate the differential, statistically relevant gene expression patterns found with neointima to patterns eventually contributed by peripheral blood cells of the patients and cultured human CASMCs, i.e., cells that are most frequently encountered in restenotic tissue(Komatsu, loc. cit.). With respect to neointima expression, the 224 aberrantly regulated genes fell into four subgroups (FIG. 14). Group I lists 62 genes that were overexpressed in neointima and not highly or detectably expressed in control vessels, CASMCs or blood cells (FIG. 14A). In group II, 43 genes are listed that are similarly expressed in neointima and CASMCs, suggesting that this gene cluster in neointima was contributed by proliferating SMCs (FIG. 5B). In group III, 62 genes are listed that are similarly expressed in neointima and blood cells, suggesting that this gene cluster was contributed to that of neointima by infiltrated blood cells (FIG. 14C). This notion is supported by the expression in group III of the largest number of genes related to inflammation in all four groups. Lastly, in group IV, 56 genes are listed that are downregulated in neointima compared to the control group (FIG. 14D).

Upregulation of γ-IFN-related Genes in Neointima

A surprising feature of the human neointima transcriptome was the apparently coordinate upregulation of 32 genes related to IFN-γ signaling (FIG. 16). The IFN-γ receptor alpha was expressed in neointima, proliferating CASMCs and—to a lesser degree—in blood cells; whereas the IFN-γ receptor beta was mainly expressed in neointima specimen. Consistent with an activation of IFN-γ signaling, upregulation of two transcription factors in neointima was found that are essential for IFN signalling: IRF-1 and ISGF3γ (p48) (FIGS. 14, 15, 16). These transcription factors are known to be transcriptionally upregulated by IFN-γ, and both are key players in IFN-γ signalling. Likewise, upregulation of the tyrosine kinase was observed Pyk2 (FIG. 16), which has been shown to play a role in the signal transduction by angiotensin in SMCs (Sabri, Circ. Res. 83 (1998), 841-851). Pyk2 is selectively activated by IFN-γ and inhibition of Pyk2 in NIH 3T3 cells results in a strong inhibition of the IFN-γ-induced activation of MAPK and STAT1.

A key event in IFN-γ-induced growth inhibition and apoptosis is the induction of caspases(Dai, Blood 93 (1999), 3309-3316). In the here presented analysis on upregulation of the IFN-γ-regulated genes for caspase-1, caspase-8 and DAP-1 in human neointima. However, mRNAs for the the anti-apoptotic proteins BAG-1, Pim-1 (both regulated by IFN-γ) and BCL-2-related protein A1 were also upregulated in neointima versus control (FIG. 16), supporting the notion that proliferation and apoptosis occur simultaneously in human neointima with a preponderance of proliferation.

Coordinated regulation of genes whose products act at different steps in the neointima process was a recurring theme of our gene expression analysis. Regarding the IFN-γ pathway, not only the genes for the complete receptor, the main transcription factors, components of the signal transduction pathway (Dap-1, BAG-1, Pim-1, IFN-γ-inducible protein, IFN-inducible protein 9-27) were induced but also several target genes of the IFN-γ pathway, like CD40, CD13 and thrombospondin-1 (FIG. 16).

The IFN-γ-regulated gene cluster was expressed in the neointima specimen but some of the relevant genes, like IRF-1, were also expressed in blood samples. To identify the cell type that predominantly contributed to the IFN-γ regulated pattern, frozen sections of neointima specimen from coronary in-stent restenosis (n=3) and from restenosis of peripheral arteries (n=6) were stained with antibodies specific for IRF-1. This protein was chosen because it is an essential component of the IFN-γ signal transduction pathway (Kimura, Genes Cells 1 (1996), 115-124) and was expressed coordinately with the other genes in the cluster (FIG. 17). Immunohistochemical analysis showed strong nuclear and cytoplasmic staining of IRF-1 in neointimal SMCs of a carotid restenosis (FIG. 17B) and of coronary in-stent restenosis (FIG. 18C), as identified by their spindle-shaped nuclei and by staining with the smooth muscle cell marker alpha-actin (FIG. 18B). The nuclear staining of IRF-1 in in-stent restenosis (FIG. 18C) indicated that the IRF-1 transcription factor is also activated. SMCs in control media of carotid arteries did not show IRF-1 staining (FIG. 17B). CD3-positive cells were much less abundant in the specimen (FIG. 18C) than SMCs (FIG. 18D), indicating that SMCs contributed mostly to the increased IRF-1 expression in human neointima.

Definition of Culturing Conditions in Order to Adapt Transcriptome Profile to That of Restenotic Tissue: IFN-γ

To adapt the transcriptional profile of cultured human coronary artery smooth muscle cells (CASMC) (Clonetics) to that of neointima, CASMC were stimulated with IFN-g and performed transcriptome analysis as described above. CASMC were cultured as described in the manufacter's manual in growth medium until 50% confluency was reached. Afterwards cells were stimulated with 1000 U/ml IFN-γ (R&D, Germany) for 16 hours at 37° C. Cells were washed twice in PBS and RNA preparation, cDNA synthesis and amplification and transcriptome analysis was performed as described above.

As shown in FIG. 19 the neointima-specific IFN-γ gene expression pattern could be generated by incubation of CASMCs with 1000 U/ml IFN-γ.

Definition of the Transcriptome/gene Expression Pattern of Neointima After Incubation With an IFN-γ Antagonist

Microscopic specimen of in-stent restenotic tissue were incubated with an antagonist for IFN-γ for different times and transcriptome analysis was performed as described. Transcriptome of treated neointima was compared to the transcriptome of untreated neointima and healthy control tissue, to measured the therapeutic effect of IFN-γ antagonists.

Definition of the Transcriptome/gene Expression Pattern of Neointima After Incubation With Rapamycin

It has been shown in the literature, that rapamycin, a ligand of the intracellular protein FKBP12 inhibits migration and proliferation of smooth muscle cells and is able to reduce neointimal hyperplasia in a porcine model of restenosis. As significant upregulation of FKBP12 in the neointima specific transcriptome was found in order to evaluated the therapeutic effect of rapamycin.

As proliferating CASMC overexpress FKBP12 like neointima, this cell line can be employed as a potential surrogate cell line for neointima in respect to therapeutic effects of rapamycin. Therefore, in a first step, CASMC were incubated with lo0ng/ml rapamycin (Sigma) for 24 hours and transcriptome analysis was performed in order to monitore the therapeutic effect.

Afterwards, microscopic specimen of in-stent restenotic tissue are incubated with rapamycin and transcriptome analysis was performed as described herein above. Transcriptome/gene expression pattern of rapamycin treated CASMC was compared to the transcriptome of rapamycin-treated neointima to measured the effectiveness of CASMC as a surrogate cell line for neointima. Tumorsuppressor genes and proliferation-inhibiting genes have upregulated in said CASMCs; therefore said CASMCs can be considered as an true surrogate for neointima.

### EXAMPLE IX

### Anti-apoptotic Effect of IFNγ on Smooth Muscle Cells

The effect of IFNγ on the survival of cultured proliferating SMCs was analyzed by flow cytometry. For this reason primary human coronary smooth muscle cells were obtained from CellSystems (Germany) and were grown in Smooth muscle cell growth medium (CellSystems) containing 5% fetal calf serum (CellSystems) at 37° C. in a humidified atmosphere of 5% CO₂. SMCs were used between passages 2 and 4. Treatment with 1000 U/ml rh-IFNγ (R&D Systems) was performed for 16 h. For induction of cell death, SMCs were incubated at 37° C. for 1 h in HBSS containing 100 μmol/l H₂O₂ and 100 μmol/l ferrous sulfate. Afterwards the cells were further cultured in freshly prepared culture medium for 8 h. Cells were labelled with FITC-labelled Annexin V (Roche Diagnostics) and propidium iodide (PI) according to the manufacturer's instructions. 10⁴ events were analyzed with a flow cytometer (Becton Dickinson).

Flow cytometric analysis revealed an anti-apoptotic effect of IFNγ on SMCs (FIG. 20). FACS analysis after double staining with PI and FITC-labeled Annexin V and showed a reduction of spontaneous apoptosis from 10% to 6% after treatment with IFNγ. The effect became more prominent after induction of apoptosis in SMCs with H₂O₂. Treatment with IFNγ reduced the number of apoptotic cells from 54% to 27%. These results clearly show that IFNγ exerts an anti-apoptotic effect on SMCs.

### EXAMPLE X

### Inhibitory Effect of IFNγ on Neointima Formation in a Mouse Model for Restenosis

To examine the vascular proliferative remodeling after carotid ligation, the mouse blood flow cessation model (Kumar, Circulation 96 (1997), 4333-4342) was used. This model is characterized by vascular proliferation of SMCs in response to ligation of the common carotid artery near bifurcation. In order to investigate the effect of an IFN-γ receptor null mutation on the development of neointima in a mouse model of restenosis IFN-γR^{-/-} knockout mice were used. Adult male 129/svJ mice (N=16) and IFN-γR^{-/-} mice (n=11) were anaesthetized by intraperitoneal injection of a solution of xylazine (5 mg/kg body weight) and ketamine (80 mg/kg body weight) and the left common carotid artery was ligated near bifurcation. After 4 week animals were reanaesthetized, sacrificied and fixed for 3 min by perfusion with 4% paraformaldehyde in 0.1 mol/l sodium phosphate buffer (pH 7.3). After excision of the left carotid arteries, vessels were fixed by immersion in 70% ethanol. Carotid arteries were embedded in paraffin and serial sections (% μm thick) were cut.

Morphometric analysis was performed on v.-Giesson stained cross sections at a distance of 600 μm from the ligation site. Digitized images of the vessels were analyzed using the image analysis software SCION image 4.0.2. Media thickness was obtained as the differences in diameter between the external and internal elastic lamina, and neointima thickness as the difference between internal elastic lamina and lumen diameter. Data from morphometric analyses are reported as mean ±SEM for the two groups of mice and tested by the t-test for unpaired samples. A p value <0.05 was regarded as significant. All analyses were performed with the use of the SPSS statistical package (version 8.0).

Substantial wall thickening due to media proliferation and neointima formation was observed in 16 wild-type mice at 4 weeks after ligation (FIG. 21). In 11 IFN-γR^{-/-} mice medial plus neointimal thickening was significantly reduced shown as mean ±SEM and analyzed by the t-test for unpaired samples. Corresponding to the reduction in proliferative responses, 11 IFN-yR^{-/-} mice had a significantly larger lumen diameter of the treated carotid segment than wild-type mice (108±15 um versus 91±24 um and p=0.033).

1. Use of a an inhibitor for the interferon-γ signaling pathway for the preparation of a pharmaceutical composition for the treatment or prevention of restenosis.

2. The use of claim 1, wherein said restenosis comprises restenosis of coronary arteries, carotid arteries, femoralis arteries, aorta-coronary vein bypass, arterial bypass, and/or of veinous bypass.

3. Use of a an inhibitor for the interferon γ signaling pathway for the preparation of a pharmaceutical composition for the prevention of restenotic modifications before, during and/or after balloon angioplasty and/or stent implantation.

4. The use of any one of claims 1 to 3 wherein said restenosis or restenotic modification is in-stent restenosis.

5. The use of any one of claims 1 to 4, wherein said inhibitor targets a gene and/or a gene product selected from the group consisting of IFN-γ allograft inflammatory factor 1, APO-2 ligand (TRAIL), BCL-2 binding athanogene-1, C5a anaphylatoxin receptor, γ-interferon-inducible protein IP-30, interferon-γ receptor, interferon-γ receptor beta, interferon-induced 56 kDa protein, interferon-inducible protein 9-27, interferon regulatory factor-1, interferon regulatory factor-7, ISGF3-γ, lymphocyte antigen, p47-PHOX, pim-1 proto-oncogen, PYK2, and thrombospondin 1.

6. The use of any one of claims 1 to 5, wherein said inhibitor is an antibody or a functional derivative or functional fragment thereof, an aptamer, a riboyzyme, an anti-sense oligonucleotide, a DNA binding protein, a peptide, a protein or histamine.

7. The use of any one of claims 1 to 5, wherein said inhibitor is selected form the group consisting of inhibitors of IFN-γ production, APO-2 ligand (T1RAIL) transcription, inhibitors of C5a anaphylatoxin receptor, inhibitors of p47-phox, inhibitors of PYK2 and inhibitors of thrombospondin-1.

8. A method for the identification of an inhibitor of the IFN-γ signaling pathway, comprising the steps of:
a. culturing a cell line in the presence of IFN-γ or a functional derivative thereof and in the presence of a test compound or a sample comprising a plurality of test compounds under conditions which permit IFN-γ signaling; and
b. detecting and/or verifying a test compound or a sample comprising a plurality of test compounds, which is capable of a suppression of said IFN-γ signaling pathway

9. The method of claim 8 wherein said detection and/or verification step is carried out by transcriptome analysis.

10. A method for preventing and/or treating restenosis in a subject comprising the step of administering to a subject suffering from said restenosis and/or to a subject susceptible to suffer from said restenosis an effective amount of an inhibitor for the interferon-γ signaling pathway as defined in any one of claims 5 to 7.

11. A method for prevention of restenotic modification before, during and/or after balloon angioplasty and/or stent implantation in a subject comprising the step of administering to said subject an effective amount of an inhibitor for the interferon γ signaling pathway as defined in any one of claims 5 to 7 before, during and/or after balloon angioplasty and/or stent implantation.

12. The method of claim 10 or 11, wherein said subject is human.
